(19) United States
(12) Patent Application Publication
(10) Pub. No.: US 2022/0033786 A1
(43) Pub. Date: Feb. 3, 2022

(54) PROVISION OF MALONYL-COA IN CORYNEFORM BACTERIA AND METHOD FOR PRODUCING POLYPHENOLES AND POLYKETIDES WITH CORYNEFORM BACTERIA

(71) Applicant: Forschungszentrum Jülich GmbH, Jülich (DE)

(72) Inventors: Jan Marienhagen, Aachen (DE); Lars Milke, Cologne (DE); Nicolai Kallscheuer, Dueren (DE); Michael Bott, Juelich (DE)

(21) Appl. No.: 17/285,539

(22) PCT Filed: Sep. 21, 2019
(86) PCT No.: PCT/DE2019/000248

§ 371 Date: Apr. 15, 2021

(30) Foreign Application Priority Data
Oct. 26, 2018 (DE) 10 2018 008 670.5

(51) Int. Cl. C12N 9/10; C12N 9/00; C12P 7/22; C12P 17/06

(52) U.S. Cl. CPC C12N 9/1029; C12Y 203/01085; C12Y 203/03001; C12P 17/06; C12N 9/1025; C12N 9/93; C12P 7/22; C12Y 604/01002

(57) ABSTRACT

A coryneform bacteria cell with an increased provision of Malonyl-CoA compared to its archetype, wherein the regulation and/or expression of one or more of genes fasB, gltA, accBC and accD1, and/or the functionality of the enzyme encoded by each gene is modified in a targeted manner. The cell may have one or more targeted modifications, including reduced or eliminated functionality of the fatty acid synthase FasB, mutation or partial or complete deletion of the fatty acid synthase encoding gene fasB, and/or reduced functionality of the promoter operatively linked to the citrate synthase gene gtIA, among other targeted modifications.

### CROSS-REFERENCE TO PRIOR APPLICATIONS

This application is a U.S. National Phase application under 35 U.S.C. § 371 of International Application No. PCT/DE2019/000248, filed on Sep. 21, 2019, and claims benefit to German Patent Application No. 10 2018 008 670.5, filed on Oct. 26, 2018. The International Application was published in German on Apr. 30, 2020 as WO 2020/083415 A1 under PCT Article 21(2).

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: 212,316 bytes ASCII (Text) file named “Updated818117_ST25,” created Sep. 14, 2021.

### FIELD

The present invention relates to a system for providing (producing) malonyl-CoA in coryneform bacteria. The present invention also relates to a method for producing secondary metabolites, such as, for example, polyphenols and polyketides with coryneform bacteria.

### BACKGROUND

A large number of very different molecules from the groups of the polyphenols (stilbenes, flavonoids) and the polyketides represent economically interesting secondary metabolites with the potential for pharmacological application. For example, stilbene resveratrol is predicted to have anti-tumor, anti-bacterial, anti-inflammatory and anti-aging effects (Pangeni et al. 2014; https://doi.org/10.1517/17425247.2014.919253).

The effect in the prevention of cardiovascular diseases is also discussed. Similar effects to anti-mutagenic, anti-oxidative, anti-proliferative and anti-atherogenic activity are described for flavonoids such as naringenin or derivatives thereof (Erlund, 2004; https://doi.Orq/10.1016/i.nutres.2004.07.005, Harbone, 2013; https://doi.Org/10.1007/978-1-4899-2915-0).

However, the natural producers of these substances (plant, fungi, bacteria) either form and accumulate only very small quantities of product, or can be cultured with difficulty or not at all. Extraction from plants in particular is economically uninteresting. Microbial production of pharmacologically and/or biotechnologically interesting polyphenols and/or polyketides on a large technical scale is therefore desirable.

The production of secondary metabolites with the bacterium E. coli and the yeast Saccharomyces cerevisiae has been described (Xu et al; 2011, https://doi.Orq/10.1016/i.vmben.2011.06.008; Li et al; 2016, https://doi.org/10.1038/srep36827). However, there are known concerns for safety in the use of E. coli for the production of such complex secondary metabolites and in particular their use in medicine. The use of a GRAS microorganism (Generally Recognized As Safe) which additionally already represents an industrially proven cell factory, is therefore very desirable.

A decisive building block for the synthesis of the polyphenols or polyketides is malonyl-CoA. While 3 Mol of malonyl-CoA/mol of product are required for representatives of the group of flavonoids and stilbenes, polyketides are constructed almost exclusively on the basis of malonyl-CoA units. Malonyl-CoA is a central intermediate in the metabolism of bacteria which cannot be transported through the cell membrane, so that extracellular feeding is not possible in a microbial production process. Although malonyl-CoA is formed by carboxylation of acetyl-CoA, the end product of glycolysis, in bacteria cells, microorganisms convert malonyl-CoA almost exclusively to fatty acid synthesis, which counteracts increased provision. In fact, fatty acid synthesis represents a very cost-intensive synthesis for the cell, so that consequently the synthesis of malonyl-CoA is strictly regulated.

An indirect means for increasing the intracellular concentration of malonyl-CoA in microorganisms is, for example, the addition of inhibitors of fatty acid synthesis, such as, for example, cerulenin. The production of resveratrol with Corynebacterium glutamicum is also described in Kallscheuer et al. (2016, https://doi.Org/10.1016/j.ymben.2016.06.003). Here too, cerulenin is used to inhibit fatty acid synthesis in order to achieve the formation of resveratrol. A significant disadvantage of the cerulenin addition, however, is that the cells completely stop their growth after the addition of cerulenin. This in turn is negative for malonyl-CoA provision (production) in the cell, which occurs only upon growth.

Cerulenin is an antibiotic that selectively inhibits fatty acid synthesis irreversibly (Omura et al; 1976; PMID 791237). By this inhibition, malonyl-CoA is no longer consumed for endogenous synthesis of fatty acids and could be available for other conversion such as for the synthesis of secondary metabolites. However, cerulenin is very expensive and therefore would hardly be suitable for use in a large-scale technically or industrially interesting microbial production method. A much more significant disadvantage of cerulenin is furthermore that the cells are extremely inhibited in their growth by the inhibition of fatty acid synthesis and as a rule can no longer grow at all after a short time (one cell division). The use of cerulenin in a microbial or biotechnological production method therefore does not represent a viable economic alternative in view of the high costs and not further optimizable yields caused by cell death.

### SUMMARY

Provided herein in an embodiment is a coryneform bacteria cell with an increased provision of Malonyl-CoA compared to its archetype, wherein the regulation and/or expression of one or more of genes fasB, gltA, accBC and accD1, and/or the functionality of the enzyme encoded by each gene is modified in a targeted manner. Also provided herein are a method for the increased provision of malonyl-CoA in coryneform bacteria and a method for the microbial production of polyphenols or polyketides in coryneform bacteria.

### BRIEF DESCRIPTION OF THE DRAWINGS

Table 1 shows an overview of bacterial strains including embodiments of the present invention.

Table 2 shows an overview of plasmids including embodiments of the present invention.

Table 3 shows an overview of the SEQ ID NOs including embodiments of the present invention.

FIG. 1 shows plasmid pK19mobsacB-fasß-E622 for amino acid substitution E622K in the fasB gene (cg2743) coding a fatty acid synthase FasB with reduced functionality.

FIG. 2 shows plasmid pK19mobsacB-fasß-G1361 D for the amino acid substitution G1361 D in the fasB gene (cg2743) coding a fatty acid synthase FasB with reduced functionality.

FIG. 3 shows plasmid pK19mobsacB-fasß-G2153D for the amino acid substitution G2153D in the fasB gene (cg2743) coding a fatty acid synthase FasB with reduced functionality.

FIG. 4 shows plasmid pK19mobsacB-fasß-G2668S for the amino acid substitution G2668S in the fasB gene (cg2743) coding a fatty acid synthase FasB with reduced functionality.

FIG. 5 shows plasmid pK19mobsacB-AfasB for the in-frame deletion of fasB (cg2743) for a fatty acid synthase FasB whose functionality is turned off.

FIG. 6 shows plasmid pK19mobsacB-P_{gltA}:P_{tiapA}-C7 for the chromosomal integration of the gene 4cl optimized for C. glutamicum codon from Petroselinum crispum under control of the IPTG inducible T7 promoter to the deletion locus Acg0344-47 (Acg 0344-47::P_{T7}-4clp_{cCg}).

FIG. 7 shows plasmid pK19mobsacB-mufasO-accBC for the mutation of the FasO binding site prior to the genes accBC (cg0802), coding for an acetyl-CoA carboxylase subunit.

FIG. 8 shows plasmid pK19mobsacB-mufasO-accD7 for the mutation of the FasO binding site prior to the gene accD1 (cg0812) coding for an acetyl-CoA carboxylase subunit, taking into account the ATG start codon and the amino acid sequence of accD1.

FIG. 9 shows plasmid pMKEx2-sts_{Ah}-4cl_{Pc} for the expression of the codon-optimized genes for C. glutamicum for a stilbene synthase (sts) from Arachis hypogea and a 4-coumarate-CoA ligase (4cl) from Petroselinum crispum under control of the IPTG inducible T7 promoter

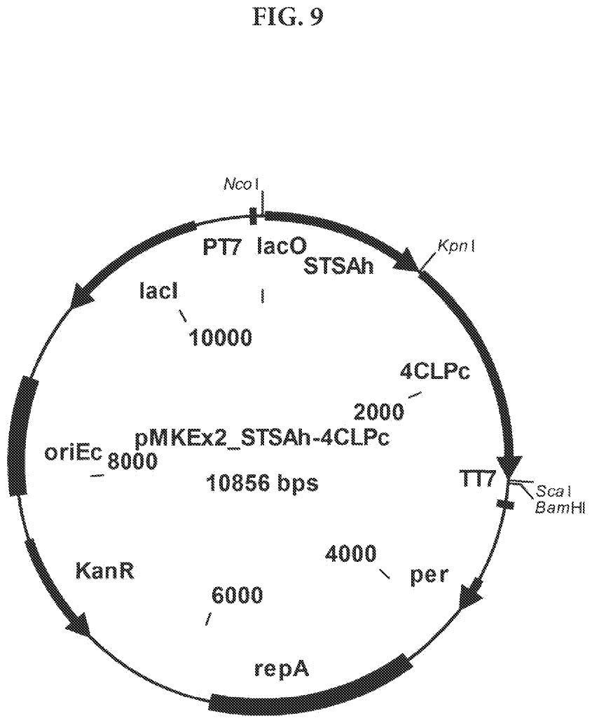

FIG. 10 shows plasmid pMKEx2-chs_{Ph}-chi_{Ph} for the expression of the codon-optimized genes for C. glutamicum for a chalcone synthase (chs) from Petunia x hybrida and a chalcone isomerase (chi) from Petunia x hybrida under control of the IPTG inducible T7 promoter.

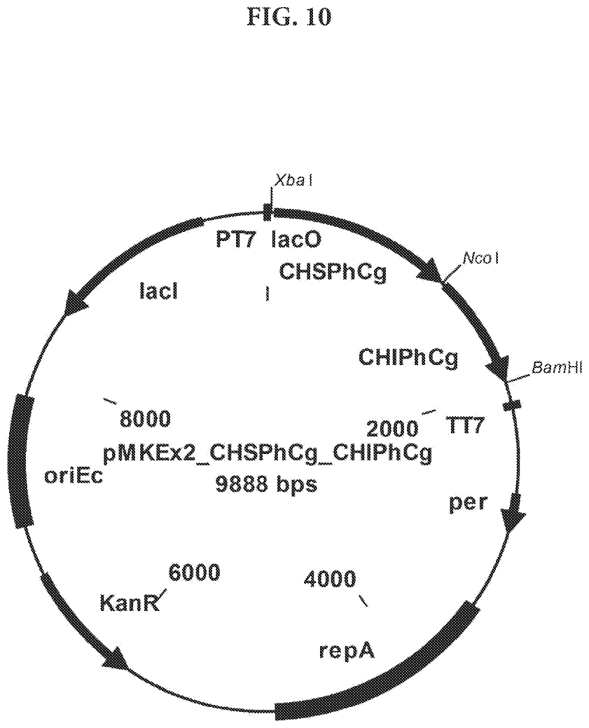

FIG. 11 shows pMKEx2-pcs_{Aa}-short for the expression of a truncated variant of the codon-optimized gene for C. glutamicum for a pentaketide chromone synthase (pcs) from Aloe arborescens

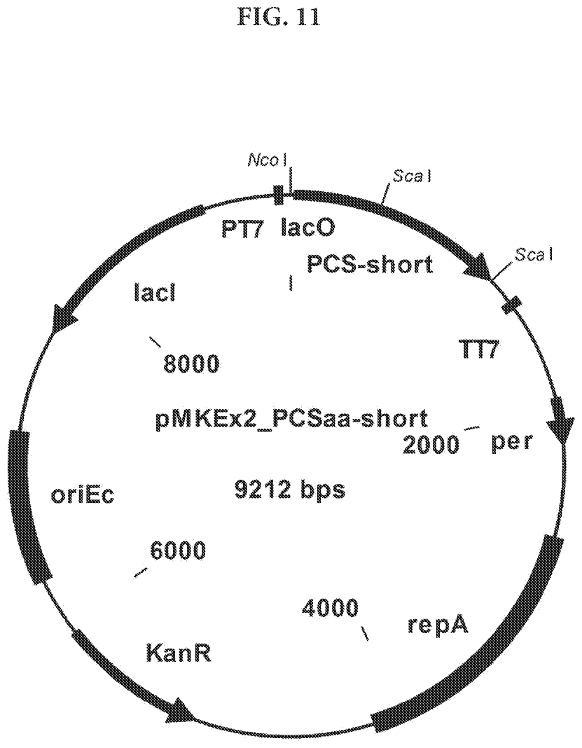

FIG. 12 shows plasmid pK19mobsacB-cg0344-47-del with which the phdBCDE operon (cg0344-47) coding for genes involved in the catabolism of phenylpropanoids, such as p-cumaric acid, is deleted from the genome.

FIG. 13 shows plasmid pK19mobsacB-cg2625-40-del with which the cat, ben and pca genes (cg2625-40) essential for the degradation of 4-hydroxybenzoate, catechol, benzoate and protocatechuate are deleted from the genome.

FIG. 14 shows plasmid pK19mobsacB-Acg0344-47::P T₇-4cl_{Pc} for the chromosomal integration of codon-optimized variant for C. glutamicum of the 4cl gene from Petroselinum crispum under control of the T7 promoter (PT7-4Cl_{Pc}) to the deletion locus Dcg0344-47.

FIG. 15 shows plasmid pK19mobsacB-cg0502-del with which the gene qsuB (cg0502) that is essential for the accumulation of protocatechuate is deleted from the genome.

FIG. 16 shows plasmid pK19mobsacB-cg1226-del with which the phobA gene (cg1226) coding for 4-hydroxybenzoate-3-hydroxylase and essential for the degradation of 4-hydroxybenzoate, catechol, benzoate and protocatechuate is deleted from the genome.

FIG. 17 shows plasmid pEKEx3-aro/-/_{£c}-ta/_{Fj}c_{g} with the genes coding for a feedback-resistant 3-deoxy-D-arabinoheptulosonate-7-phosphate synthase (aroH), preferably from E. coli (aroH_{εc}), and for a tyrosine ammonium lyase (tal), preferably from Flavobacterium johnsoniae (tal_{Fj}), that is adapted for codon use of C. glutamicum. This plasmid is used in the synthesis of polyphenols or polyketides on growth starting from glucose.

FIG. 18 shows plasmid pMKEx2_sfS_{Al},_4c/_{Pc}, for the expression of the gene sts from Arachis hypogea (stS_{Ah}) and 4c/ from Petroselinum crispum (4c/_{Pc}) in coryneform bacteria cells.

FIG. 19 shows plasmid pMKEX2-cf/s_{Pl},-ch/_{Pl} for the expression of the genes chs and chi from Petunia x hybrida (chs_{pH} and chipₕ) in coryneform bacteria cells.

FIG. 20 shows plasmid pMKEx2_pcs_{Aa} for the expression of pcs from Aloe arborescens (pcs_{Aa}) with adaptation to codon usage of coryneform bacteria cells.

FIG. 21 shows pMKEx2_pcs_{Aa}-short for the expression of the gene variant of pcs from Aloe arborescens (pcs_{Aa}) in coryneform bacteria cells.

FIG. 22 shows a sequence comparison of the native promoter region P_{daP}A of the C. glutamicum wild-type gene with the P_{dapA} 07 promoter according to an embodiment of the invention replacing the native gtlA promoter prior to the gtlA gene from Corynebacterium glutamicum according to the invention. The promoter region PgltA::PdapA-C7 according to an embodiment of the invention has, in addition to a replacement of the promoter region of gtlA (PgtlA) with the promoter of dapA (PdapA), additional nucleotide substitutions at positions 95 (a->t) and 96 (g->a) prior to the start codon ATG of gtlA.

FIG. 23 shows an overview of the fasO binding sites 5-operably linked prior to the genes accBC and accD1 with nucleotide substitutions according to an embodiment of the invention resulting in a loss of binding of the fasR regulator and increased functionality or expression of the accBCD1 genes. An overview of FasO accD1 sequences is also shown. The accD1 start codon: underlined (AS sequence correspondingly translated from here) gray background: conserved regions of the fasO binding motive which have to be mutated in order to prevent FasR binding red: differences to the native sequence.

FIG. 24 shows a diagram with malonyl-CoA concentrations (measured in the form of μM malonate) in coryneform bacteria cells according to an embodiment of the invention.

### DETAILED DESCRIPTION

In an embodiment, the present invention provides a system and method for increasing the concentration of the central metabolite malonyl-CoA in coryneform bacteria which is independent of the addition of cerulenin.

In an embodiment, the present invention provides an economically interesting system which is suitable for the biotechnological provision of malonyl-CoA in coryneform bacteria and in which the growth of the cells remains unaffected or is not negatively influenced or does not occur at all.

In an embodiment, the present invention avoids such interference with the metabolism of coryneform bacteria which may have widely undefined physiological effects, as is the case, for example, when one or more centrally acting regulators (such as the regulator protein FasR), which exert influence on a multiplicity of genes or proteins in a cell, are turned off. Thus, a further object of the present invention is to provide a specifically created and exactly defined cell system and one or more defined homologous structural elements that allow the microbial preparation of malonyl-CoA with non-recombinant coryneform bacteria (non-GVO) while simultaneously overcoming known disadvantages.

In an embodiment, the present invention provides a method for the microbial production of economically interesting secondary metabolites, such as molecules from the groups of polyphenols (stilbenes, flavonoids) and the polyketides, in coryneform bacteria, in which the known disadvantages are overcome.

There first follows a brief description of the present invention, without the subject matter of the invention being limited thereby.

An embodiment of the present invention is a coryneform bacteria cell with an increased provision of Malonyl-CoA compared to its archetype, in which the regulation and/or expression of the genes selected from the group comprising fasB, gltA, accBC and accD1 and/or the functionality of the enzymes encoded by them is modified in a targeted manner. A further embodiment of the invention comprises a coryneform bacteria cell which has one or more purposeful modifications selected from the group comprising
a) Reduced or eliminated functionality of the fatty acid synthase FasB;
b) Mutation or partial or complete deletion of the fatty acid synthase-encoding gene fasB;
c) Reduced functionality of the promoter operatively linked to the citrate synthase gene gtlA;
d) Reduced expression of the gene gltA coding for the citrate synthase CS;
e) Reduced or eliminated functionality of the operator binding sites (fasO) for the regulator FasR in the promoter regions of the genes accBC and accD1 coding for the acetyl-CoA carboxylase subunits;
f) Derepressed expression of the genes accBC and accD1 coding for the acetyl-CoA carboxylase subunits;
g) One or more combinations of a)-f).

Included in the scope of the invention is thus also a coryneform bacteria cell in which the functionality of the fatty acid synthase FasB is reduced or turned off and/or the gene fasB coding for the fatty acid synthase is purposefully mutated, preferably by one or more nucleotide substitutions, or is partially or completely deleted.

Also included in the scope of the invention is a coryneform bacteria cell for which the expression of the gene gltA coding for the citrate synthase is reduced by mutation, preferably several nucleotide substitutions, of the operatively linked promoter.

Another embodiment of the present invention is a coryneform bacteria cell for which the functionality of the operator binding sites (fasO) for the regulator FasR in the promoter regions of the genes accBC and accD1 coding for the acetyl-CoA carboxylase subunits, preferably by one or more nucleotide substitutions, is reduced or turned off and the expression of the genes accBC and accD1 coding for the acetyl-CoA carboxylase subunits is derepressed, preferably increased.

Another embodiment of the present invention is a coryneform bacteria cell that comprises a combination of reduced expression and/or activity of the citrate synthase (CS) and deregulated, increased expression and/or activity of the acetyl-CoA carboxylase subunits (AccBC and AccD1).

Included in the scope of the invention is also a coryneform bacteria cell that comprises a combination of reduced expression and/or activity of the citrate synthase (CS) and deregulated, increased expression and/or activity of the acetyl-CoA carboxylase subunits (AccBC and AccD1) and reduced or eliminated functionality of the fatty acid synthase FasB.

Another embodiment of the present invention is furthermore a coryneform bacterial cell for producing polyphenols or polyketides, which has modifications of the aforementioned type and in which the catabolic pathway of aromatic components, preferably selected from the group comprising phenylpropanoids and benzoic acid derivatives, is additionally turned off.

Included in the scope of the invention is furthermore a coryneform bacteria cell that additionally comprises genes coding for a feedback-resistant 3-deoxy-D-arabinoheptulosonate-7-phosphate synthase (aroH), preferably from E. coli, and for a tyrosine ammonium lyase (tal), preferably from Flavobacterium johnsoniae.

Another embodiment of the present invention is also a coryneform bacteria cell of the aforementioned type which additionally comprises enzymes derived from plants or the genes coding them for the polyphenol or polyketide synthesis.

According to an embodiment of the invention, the coryneform bacteria cell is the genus selected from the group comprising Corynebacterium and Brevibacterium, preferably Corynebacterium glutamicum, particularly preferred Corynebacterium glutamicum ATCC13032 or their purposefully genetically modified variants.

An embodiment of the present invention is also a method for the increased provision of malonyl-CoA in coryneform bacteria with the aforementioned coryneform bacteria and to a method for the microbial production of polyphenols or polyketides in coryneform bacteria.

According to the invention, the methods are independent of the addition of cerulenin.

Another embodiment of the present invention is the use of a coryneform bacteria cell according to the invention for the increased provision of malonyl-CoA in coryneform bacteria, and to the use of a coryneform bacteria cell according to the invention for producing polyphenols or polyketides with coryneform bacteria.

Another embodiment of the invention comprises a composition comprising secondary metabolites selected from the group of the polyphenols and polyketides, preferably stilbenes, flavonoids and polyketides, particularly preferably resveratrol, naringenin and noreugenin, produced using a coryneform according to the invention or a method according to the invention. In another embodiment, the present invention includes the use of a previously mentioned composition according to the invention for producing pharmaceuticals, foodstuffs, feedstuffs and/or for use in plant physiology.

In the following, the subject matter of the invention is explained in more detail using examples and figures, without the subject matter of the invention being limited thereby.

Some definitions that are important to the understanding of the present invention precede the description of the exemplary embodiments.

An embodiment of the present invention is a coryneform bacteria cell with an increased provision of Malonyl-CoA compared to its archetype, in which the regulation and/or expression of the genes selected from the group comprising fasB, gltA, accBC and accD1 and/or the functionality of the enzymes encoded by them is modified in a targeted manner.

Thus included as an embodiment of the invention is a coryneform bacteria cell which has one or more purposeful modifications selected from the group comprising
a) Reduced or eliminated functionality of the fatty acid synthase FasB;
b) Mutation or partial or complete deletion of the fatty acid synthase-encoding gene fasB;
c) Reduced functionality of the promoter operatively linked to the citrate synthase gene gtlA;
d) Reduced expression of the gene gltA coding for the citrate synthase CS;
e) Reduced or eliminated functionality of the operator binding sites (fasO) for the regulator FasR in the promoter regions of the genes accBC and accD1 coding for the acetyl-CoA carboxylase subunits;
f) Derepressed expression of the genes accBC and accD1 coding for the acetyl-CoA carboxylase subunits;
g) One or more combinations of a)-f).

Included in the scope of the invention is also a coryneform bacteria cell in which the functionality of the fatty acid synthase FasB is reduced or turned off and/or the gene fasB coding for the fatty acid synthase is purposefully mutated, preferably by one or more nucleotide substitutions, or is partially or completely deleted.

Similarly included in the scope of the invention is a coryneform bacteria cell for which the expression of the gene gltA coding for the citrate synthase is reduced by mutation, preferably several nucleotide substitutions, of the operatively linked promoter.

The subject matter of the present invention also includes a coryneform bacteria cell for which the functionality of the operator binding sites (fasO) for the regulator FasR in the promoter regions of the genes accBC and accD1 coding for the acetyl-CoA carboxylase subunits, preferably by one or more nucleotide substitutions, is reduced or turned off and the expression of the genes accBC and accD1 coding for the acetyl-CoA carboxylase subunits is derepressed, preferably increased.

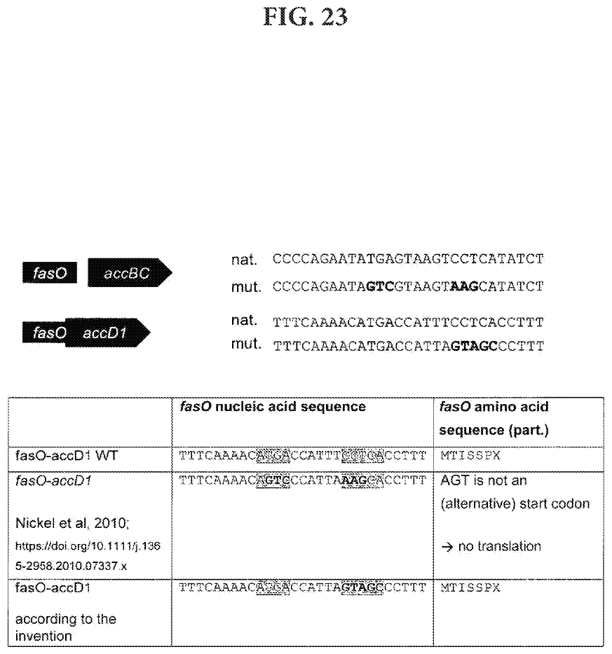

Mutations of the fasO binding site upstream of accBC and accD1 are known (Nickel et al., 2010; https://doi.Org/10.1111/j.1365-2958.2010.07337.x). Here, mutations of the fasO binding site are described which lead to a loss of the binding of the fatty acid synthesis regulator FasR. In the case of accBC, the fasO binding site is upstream of the accBC gene so that the mutation of (Nickel et al., 2010) could be adopted. In the case of accD1, the reading frame and fasO binding site overlap (FIG. 23; ATG in the left, gray shaded box is the start codon of accD1). A mutation in this region is therefore not possible, since otherwise the start codon would be mutated here. Since also no alternative start codon (GTG or TTG) is formed by the mutation, translation is not possible with the consequence that there is no AccD1 subunit and thus no functional acetyl-CoA carboxylase activity. This has the further consequence that malonyl-CoA cannot be formed and the cells are presumably lethal or strongly crippled. Thus, the Nickel et al. mutations are not suitable for the present invention.

According to an embodiment of the invention, a novel fasO binding site 5′-operatively linked upstream of the accD1 gene of coryneform bacteria is provided. This is distinguished in an advantageous manner in that, taking into account the amino acid sequence and the best possible codon usage in coryneform bacteria, they have a maximum deviation from the native fasO sequence: MTISSPX (FIG. 23). Nucleotide substitutions are present in the fasO binding site upstream of accBC at positions 11-13 (tga->gtc) and 20-22 (cct->aag). Nucleotide substitutions are present at positions 20-24 (cctca->gtacg) in the fasO binding site upstream of accD1. In an embodiment of the present invention, the fasO binding sites according to the invention have a nucleic acid sequence upstream of the genes accBD and accD1, according to SEQ ID NO: 13 and 15, respectively.

Another embodiment of the present invention is a coryneform bacteria cell that comprises a combination of reduced expression and/or activity of the citrate synthase (CS) and deregulated, increased expression and/or activity of the acetyl-CoA carboxylase subunits (AccBC and AccD1).

Included in the scope of the invention is also a coryneform bacteria cell that comprises a combination of reduced expression and/or activity of the citrate synthase (CS) and deregulated, increased expression and/or activity of the acetyl-CoA carboxylase subunits (AccBC and AccD1) and reduced or eliminated functionality of the fatty acid synthase FasB.

The coryneform bacteria cell according to an embodiment of the invention is characterized in particular in that the anabolism of malonyl-CoA is deliberately increased and at the same time the growth of the cell is unaffected. Such a coryneform bacteria cell has not been described so far. Conventionally, to increase the malonyl-CoA concentration in the cell, the catabolic metabolism of malonyl-CoA is switched off, but this has the negative effect that the cells can no longer grow. This is described in a variety of ways, for example by the addition of cerulenin. However, poor growth negatively affects the strictly controlled malonyl-CoA provision, i. e. less malonyl-CoA is provided, thus proving to be counter-productive. The present invention advantageously overcomes such drawbacks.

The term “archetype” is to be understood in the sense of the present invention as meaning both the “wild type” of a coryneform bacteria cell which, for example, provides a genetically unaltered starting gene or starting enzyme, and also direct derivatives thereof. Coryneform wild-type cells of the genus Corynebacterium or Brevibacterium are preferred; particular preference is given to coryneform bacterial cells of the wild type Corynebacterium glutamicum; very particular preference is given to coryneform bacterial cells of the wild type Corynebacterium glutamicum ATCC 13032. According to the invention, the term “archetype” thus includes in addition to the “wild type” also specifically derived, precisely defined and precisely characterized “derivatives” of the wild type. The “derivatives” in this case have changes which have been carried out in a targeted, directed and controlled manner by means of molecular biological methods and are homologous, non-recombinant changes, such as, for example, nucleotide substitutions or deletions or the adaptation of heterologous nucleic acid sequences to the codon usage of the wild type. The resulting derivative is well characterized physiologically and does not carry heterologous nucleic acid sequences; neither chromosomally coded nor plasmid coded. An example of an “archetype” in the sense of the present invention is a wild-type coryneform bacteria cell in which the genes responsible for the degradation of aromatic components from the genome are deleted. In addition to deletions, targeted nucleotide substitutions in the genome are also conceivable, through which the wild type genetically remains a homologous, non-recombinant organism. This example is not to be construed as limiting the present invention. Since embodiments of the invention concern targeted nucleotide exchanges of the same, homologous host organism, the resulting organism is non-recombinantly altered according to certain embodiments of the invention. In the sense of the invention, “homologous” is to be understood to mean that the enzymes according to the invention and the nucleic acid sequences coding them and the non-coding nucleic acid sequences regularly linked thereto according to the invention originate from a common starting strain of coryneform bacteria cells. According to the invention, “homologous” is used synonymously with the term “non-heterologous”. An “archetype” according to the invention is genetically and physiologically exactly characterized, homologous, non-recombinant and can be equated with the “wild type”. The terms “wild type”, “derivatives” and “archetype” are used synonymously according to the invention.

For the purposes of the present invention, a “reduced or eliminated functionality” relates, for example, both to the functionality of the protein-level fatty acid synthase FasB according to the invention and to the nucleic acid sequence coding it according to the invention. Thus, “functionality” generally comprises the function of a protein or a nucleic acid sequence coding for it, which may be reduced or turned off, for example, by nucleotide substitution or deletion. Thus, the “functionality” also comprises the activity of a protein which may be altered, such as reduced or turned off. According to the invention, the altered activity of a protein can comprise both changes in the active, catalytic center and also in the regulatory center. These variants are likewise included according to the invention.

An embodiment of the present invention comprises a coryneform bacteria cell which is characterized in that it has a modified functionality of an enzyme and/or of the coding nucleic acid sequence and/or of an operatively linked, regulatory, non-coding nucleic acid sequence. A further embodiment of a coryneform bacteria cell according to the invention is characterized in that the modification is based on changes selected from the group comprising a) modifying the regulation or signal structures for gene expression, b) modifying the transcription activity of the encoding nucleic acid sequence, or c) change of the coding nucleic acid sequence. The invention thus comprises, for examples, changes of the signal structures of the gene expression, such as by modifying the repressor genes, activator genes, operators, promoters; attenuators, ribosome binding sites, the start codon, terminators. Also included are the introduction of a stronger or weaker promoter or an inducible promoter into the genome of the inventive coryneform bacteria cell or deletions or nucleotide substitutions in coding or non-coding regions, wherein the molecular biological methods are known to the person skilled in the art. The subject matter of the present invention includes a coryneform bacteria cell in which the changes are present in the genome in chromosomally coded form or are present extrachromosomally, i.e., vector-coded or plasmid-coded. Suitable plasmids according to the invention are those replicated in coryneform bacteria. Numerous known plasmid vectors, such as pZ1 (Menkel et al., Applied and environmental Microbiology (1989) 64: 549-554), pEKExl (Eikmanns et al., Gene 102:93-98 (1991)) or pHS2-1 (Sonnen et al., Gene 107:69-74 (1991)), are based on the cryptic plasmids pHM1519, pBL1, or pGA1. Other plasmid vectors, such as those based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891), can be used in the same manner (O. Kirchner 2003, J. Biotechnol. 104:287-99). Regulatable expression vectors may also be used, such as pEKEx2 (B. Eikmanns, 1991 Gene 102:93-8; O. Kirchner 2003, J. Biotechnol. 104:287-99) or pEKEx3 (Gande, R.; Dover, L. G.; Krumbach, K.; Besra, G. S.; Sahm, H.; Oikawa, T.; Eggeling, L, 2007. “The two carboxylases of Corynebacterium glutamicum essential for fatty acid and mycolic acid synthesis.” Journal of Bacteriology, 189 (14), 5257-5264. https://doi.Orq/10.1128/JB.00254-07). The gene can also be expressed by integration into the chromosome as a single copy (P. Vasicova 1999, J. Bacteriol. 181:6188-91), or multiple copy (D. Reinscheid 1994 Appl. Environ Microbiol 60:126-132). The transformation of the desired strain with a vector is accomplished by conjugation or electroporation of the desired strain of C. glutamicum, for example. The process of conjugation is described, for example, in Schafer et al. (Applied and environmental Microbiology (1994) 60:756-759). Methods for transformation are described, for example, in Tauch et al. (FEMS Microbiological Letters (1994) 123:343-347).

In addition to preferred partial or complete deletions of coding nucleic acid sequences and/or regulatory structures according to certain embodiments of the invention, embodiments of the invention also include modifications, such as transitions, transversions, or insertions, as well as directed evolution processes. Instructions for generating such modifications can be found in known textbooks (R. Knippers “Molekulare Genetik [Molecular Genetics],” 8th edition, 2001, Georg Thieme Verlag, Stuttgart, Germany). Preferred are nucleic acid substitutions or deletions according to embodiments of the invention.

In the context of the present invention, a “reduced or eliminated functionality” refers not only to the functionality of a gene or protein, but also to an altered functionality of regulator binding sites, such as the operator binding site fasO, to which normally a centrally acting regulator protein such as, for example, fasR, binds, and thereby represses expression of the coding nucleic acid sequence. Thus, within the meaning of the present invention, “reduced” or “turned off” also means that the expression of the coding nucleic acid sequence is poorer compared to the situation in a wild-type or archetype host cell in the sense of the invention or is no longer under the expression control of the regulator. In the context of the present invention, “reduced” or “turned off” is intended to be synonymous with “deregulated” or “derepressed”. Thus, in the sense of the invention, a “derepressed functionality” of a regulator binding site can also lead to an increased expression of the relevant subsequent gene.

In the sense of the present invention, a “reduced or eliminated functionality” also refers to altered functionality of promoter regions in the 5′ regulatory region upstream of a coding gene. Alterations in “functionality” may enhance or else reduce the activity of the promoter. In a variant according to the invention, a promoter, such as, for example, upstream of the gene gtlA coding for the citrate synthase, is reduced in its function and thus activity. This has the consequence that the gene coded by this promoter is expressed weaker. The regulatory mechanisms and their effects upon alteration are familiar in all variants to the person skilled in the art.

The term “modification” within the meaning of the present invention means a “change”, also for example, a “genetic modification,” which means, according to the invention, that although a genetic engineering method is used, no insertions of nucleic acid molecules are produced. Within the meaning of the invention, “modifications” or “changes” refers to substitutions and/or deletions, preferably substitutions. Within the meaning of the present invention, “modification”, “change” or “genetic modification” is also generated in a regulatory, non-coding region of the nucleic acids according to the invention. All conceivable positions in a regulatory region of coding genes or gene clusters, the modifications of which have a measurable effect on the functionality of the fasO binding sites and fasO binding, in the sense of “reduced” or “turned off” are intended and included within the meaning of the invention.

The subject matter of the present invention also includes a protein coding for a fatty acid synthase FasB isolated from coryneform bacteria whose functionality is reduced or turned off and with which an increased provision of malonyl-CoA in coryneform bacteria is made possible, the amino acid sequence comprising at least 70% identity to the amino acid sequence selected from the group comprising SEQ ID NO. 2, 4, 6, 8 and 10 or fragments or alleles thereof. Embodiments of the invention also include a fatty acid synthase FasB with an amino acid sequences selected from the group comprising SEQ ID NO. 2, 4, 6, 8 and 10 or fragments or alleles thereof. Furthermore, a fatty acid synthase coded by a nucleic acid sequence containing at least 70% identity to the nucleic acid sequence according to SEQ ID NO. 1, 3, 5, 7 and 9 or fragments thereof is included in the scope of the invention. The present invention also includes a fatty acid synthase coded by a nucleic acid sequence selected from the group of SEQ ID NO. 1, 3, 5, 7 and 9 or fragments thereof.

Also included in the invention are proteins encoding an amino acid sequence with at least 75 or 80%, preferably at least 81, 82, 83, 84, 85, or 86% identity, particularly preferably at least 87, 88, 89, 90% identity, very particularly preferably at least 91, 92, 93, 94, 95% identity, or most preferably 96, 97, 98, 99, or 100% identity to the amino acid sequence according to SEQ ID NO. 2, 4, 6, 8 and 10 or fragments or alleles thereof. In addition, embodiments of the present invention relate to a fatty acid synthase fasB containing an amino acid sequence according to SEQ ID NO. 2, 4, 6, 8 and 10 or fragments or alleles thereof.

Subject matter of the present invention also includes a nucleic acid sequence coding for a fatty acid synthase FasB from coryneform bacteria whose functionality is reduced or turned off, selected from the group comprising:
a) a nucleic acid sequence containing at least 70% identity to the nucleic acid sequence selected from the group of SEQ ID NO. 1, 3, 5, 7 and 9 or fragments thereof,
b) a nucleic acid sequence which, under stringent conditions, hybridizes with a complementary sequence of a nucleic acid sequence selected from the group of SEQ ID NO. 1, 3, 5, 7 and 9 or fragments thereof,
c) a nucleic acid sequence selected from the group of SEQ ID NO. 1, 3, 5, 7 and 9 or fragments thereof, or
d) a nucleic acid sequence coding for a fatty acid synthase FasB corresponding to each of the nucleic acids according to a)-c) but which differs from these nucleic acid sequences according to a)-c) by the degeneracy of the genetic code or functionally neutral mutations,
   for the increased provision of malonyl-CoA in coryneform bacteria.

The subject matter of the invention also includes a fatty acid synthase fasB coded by a nucleic acid sequence containing at least 70% identity to the nucleic acid sequence according to SEQ ID NO. 1, 3, 5, 7 and 9 or fragments thereof. Also included in the invention are nucleic acid sequences which have at least 75% or 80%, preferably at least 81, 82, 83, 84, 85, or 86% identity, particularly preferably 87, 88, 89, 90% identity, very particularly preferably at least 91, 92, 93, 94, 95% identity, or most preferably 96, 97, 98, 99, or 100% identity to the nucleic acid sequence SEQ ID NO. 1, 3, 5, 7 and 9 or fragments thereof. In addition, the present invention relates to a fatty acid synthase fasB coded by a nucleic acid sequence according to SEQ ID NO. 1, 3, 5, 7 and 9 or fragments thereof.

According to an embodiment, the invention also comprises a coryneform bacteria cell which comprises a protein coding for a fatty acid synthase FasB with reduced or eliminated functionality or a nucleic acid sequence coding for a fatty acid synthase FasB having the aforementioned altered functionality.

An embodiment of the present invention also comprises a coryneform bacteria cell which has one or more purposeful modifications selected from the group comprising
a) Reduced or eliminated functionality of the fatty acid synthase FasB with at least 70% identity to the amino acid sequence selected from the group comprising SEQ ID NO. 2, 4, 6, 8 and 10 or fragments or alleles thereof;
b) Mutation or partial or complete deletion of the fatty acid synthase-encoding gene fasB with a nucleic acid sequence containing at least 70% identity to the nucleic acid sequence selected from the group SEQ ID NO. 1, 3, 5, 7 and 9 or fragments thereof;
c) Reduced functionality of the promoter operatively linked to the citrate synthase gene gltA according to SEQ ID NO. 11;
d) Reduced expression of the gene gltA coding for the citrate synthase (CS);
e) Reduced or eliminated functionality of the operator binding sites (fasO) for the regulator FasR in the promoter regions of the genes accBC and accD1 coding for the acetyl-CoA carboxylase according to SEQ ID NO. 13 and 15;
f) Derepressed expression of the genes accBC and accD1 coding for the acetyl-CoA carboxylase subunits;
g) One or more combinations of a)-f).

Also included in embodiments of the present invention are proteins of the fatty acid synthase FasB of coryneform bacteria and/or nucleic acid sequences encoding a fatty acid synthase FasB of coryneform bacteria in which nucleotide substitutions and correspondingly corresponding amino acid substitutions are present. Such embodiments are explained in the exemplary embodiments, but these do not have a limiting effect on the present invention.

In embodiments of the present invention, the functionality of the promoter operatively linked to the citrate synthase gene gltA is also reduced. For this purpose, nucleotide substitutions according to the invention can take place in the binding sites responsible for the binding of the polymerase, or an exchange of an entire promoter sequence of a weaker promoter against the naturally occurring promoter sequence can take place, or a combination of both, wherein a weaker promoter is additionally attenuated further by nucleotide substitution. Since the invention concerns targeted nucleotide exchanges of the same, homologous host organism, the resulting organism is non-recombinantly altered according to the invention.

In the sense of the invention, “homologous” is to be understood to mean that the enzymes according to the invention and the nucleic acid sequences coding them and the non-coding nucleic acid sequences regularly linked thereto according to the invention originate from a common starting strain of coryneform bacteria cells. According to the invention, “homologous” is used synonymously with the term “non-heterologous”.

The term “nucleic acid sequence” within the meaning of the present invention means any homologous molecular unit which transports genetic information. Accordingly, this relates to a homologous gene, preferably a naturally occurring and/or non-recombinant homologous gene, to a homologous transgene or codon-optimized homologous genes. The term “nucleic acid sequence” according to the invention refers to a nucleic acid sequence or fragments or alleles thereof that code or express a specific protein. Preferably, the term “nucleic acid sequence” refers to a nucleic acid sequence containing regulatory sequences that precede (upstream, 5′ non-coding sequence) and follow (downstream, 3′ non-coding sequence) the coding sequence. The term “naturally occurring” gene refers to a gene found in nature, e.g., from a wild-type strain of a coryneform bacterial cell, with its own regulatory sequences.

Within the meaning of the present invention, the term “operatively linked region” relates to an association of nucleic acid sequences on a single nucleic acid fragment so that the function of the one nucleic acid sequence is influenced by the function of the other nucleic acid sequence. In the context of a promoter or binding site for a regulator protein, the term “operatively linked” within the meaning of the invention means that the encoding sequence is under the control of the regulatory region (especially of the promoter or of the regulator binding site) which regulates the expression of the encoding sequence.

According to embodiments of the invention, a novel fasO binding site is also provided 5′-operatively linked upstream of the accD1 gene of coryneform bacteria. In variations of the present invention, a reduced or eliminated functionality of the operator binding sites (fasO) for the regulator FasR in the promoter regions of the genes accBC and accD1 coding for the acetyl-CoA carboxylase subunits is also included. This is distinguished in an advantageous manner in that, taking into account the amino acid sequence and the best possible codon usage in coryneform bacteria, they have a maximum deviation from the native fasO sequence: MTISSPX (FIG. 23). Nucleotide substitutions are present in the fasO binding site upstream of accBC at positions 11-13 (tga->gtc) and 20-22 (cct->aag). Nucleotide substitutions are present at positions 20-24 (cctca->gtacg) in the fasO binding site upstream of accD1.

The subject matter of the present invention thus also includes a nucleic acid sequence for an operatively linked fasO binding site in the regulatory, non-coding region 5′ upstream of the accD1 gene from coryneform bacteria which has the nucleotide substitutions according to SEQ ID NO. 15. Because of the fasO binding site modified in its functionality, binding of the FasR regulator protein is no longer possible and leads to a deregulation of the expression of the accD1 gene, resulting in increased expression of the subunit accD1. In combination with a deregulated, i.e. increased, expression of the subunit accBC according to certain embodiments of the invention, this leads, according to the invention, to an increased provision (production) of malonyl-CoA in coryneform bacteria.

The subject matter of the present invention also includes a coryneform bacteria cell in which the modifications according to the invention are advantageously chromosomally coded. Also included in the invention is a coryneform bacteria cell that is non-recombinant (non-GVO).

Within the meaning of the present invention, the term “non-recombinant” is understood to mean that the genetic material of the coryneform bacterial cells according to the invention is only modified in such a way that it could occur naturally, e.g., by natural recombination or natural mutation. The coryneform bacterial cells according to the invention are thus distinguished as non-genetically modified organisms (non-GMO).

This also opens up the possibility of further optimizing industrially interesting production strains of coryneform bacteria without having to introduce recombinant or heterologous gene material into the cell. Embodiments of the present invention thus provide a system by means of which the microbial production of malonyl-CoA can be carried out in a considerably simpler, more stable, cheaper, and more economical manner. This is because all hitherto known production strains with a malonyl-CoA synthesis capacity require complex media for their growth, as a result of which the cultivation becomes markedly more complex, more expensive, and thus more uneconomical. Mention must be made above all of the addition of inhibitors of fatty acid synthesis, such as, for example, cerulenin, which is very expensive and thus not suitable for use in a large-scale technical production method. All malonyl-CoA producers described so far are not GRAS organisms. This gives rise to a disadvantage for use in certain industrial sectors (e.g., food and pharmaceutical industries) as a result of complicated approval processes.

The coryneform bacteria cell according to embodiments of the invention offers a multiplicity of advantages, a selection of which is described below. Coryneform bacteria, preferably the genus Corynebacterium, are a “generally recognized as safe” (GRAS) organism, which can be used in all industrial sectors. Coryneform bacteria achieve high growth rates and biomass yields on defined media (Grianberger et al., 2012) and there is extensive experience in the industrial use of coryneform bacteria (Becker et al., 2012).

Coryneform bacteria of the genus Corynebacterium or Brevibacterium are included according to embodiments of the invention. Coryneform bacteria cell variants according to the invention are selected from the group comprising Corynebacterium and Brevibacterium, preferably Corynebacterium glutamicum, particularly preferred Corynebacterium glutamicum ATCC 13032, Corynebacterium acetoglutamicum, Corynebacterium thermoaminogenes, Brevibacterium flavum, Brevibacterium lactofermentum or Brevibacterium divaricatum. Also included in the scope of the invention is a coryneform bacterial cell selected from the group comprising Corynebacterium glutamicum ATCC13032 or purposefully modified derivatives or archetypes, Corynebacterium acetoglutamicum ATCC15806, Corynebacterium acetoacidophilum ATCC 13870, Corynebacterium thermoaminogenes FERM BP-1539, Brevibacterium flavum ATCC14067, Brevibacterium lactofermentum ATCC13869, Brevibacterium divaricatum ATCC14020.

Embodiments of the invention also include a coryneform bacteria cell with one or more of the aforementioned modifications according to the invention starting from Corynebacterium glutamicum, preferably Corynebacterium glutamicum ATCC13032, in which in addition the catabolic pathway of aromatic components, preferably selected from the group comprising phenylpropanoids and benzoic acid derivatives, is turned off.

Further embodiments of a coryneform bacteria cell according to the invention are characterized by the fact that the functionality and/or activity of the enzymes or the expression of the genes coding them are turned off by deletions of the gene clusters cg0344-47 (phdBCDE operon), cg2625-40 (cat, ben and pca), cg1226 (pobA) and cg0502 (qsuB). These cells according to the invention are purposefully modified and have not been produced by random mutagenesis. They are advantageously distinguished by the fact that they are characterized in a genetically precise manner and the said modifications are achieved by deletions. According to embodiments of the invention, these deletions are chromosomally coded. Thus, these cells have exclusively homologous DNA and are non-recombinantly altered. This distinguishes them, in addition to the feature of belonging to the GRAS organisms, advantageously for a microbial production of products, such as, for example, secondary plant metabolites. The coryneform bacteria cell according to the invention is also advantageously characterized in that it does not require extrachromosomal DNA, such as plasmids or vectors, for the increased provision of malonyl-CoA. Firstly, bacteria strains with more than 2 plasmids or more than 2 genes per plasmid are generally not stable, secondly it must be considered that the microbial production of complex secondary metabolites in bacteria comprised according to embodiments of the invention requires a heterologous expression of the corresponding plant genes for polyphenol and/or polyketide production, and thirdly these desired products or their precursors should not be decomposed by cell-specific activities, such as enzymatic degradation of aromatic components. Certain embodiments of the invention provide a system for the increased provision of malonyl-CoA in coryneform bacteria without having to carry out plasmid-coded changes while at the same time preventing the degradation of the desired aromatics-containing products and their precursors in coryneform bacteria. This highly advantageous system of a coryneform bacteria cell according to embodiments of the invention thus permits degrees of freedom as to which plant or other heterologous genes can be introduced extrachromosomally into the system, in order to thus enable a stable microbial production of plant secondary metabolites.

The subject matter of the present invention also includes a coryneform bacteria cell characterized in that it provides an increased intracellular concentration of malonyl-CoA irrespective of the addition of fatty acid synthesis inhibitors. This increased provision of malonyl-CoA as a central intermediate can be used according to embodiments of the invention for the preparation of products for the synthesis of which an increased concentration of malonyl-CoA is required, such as, for example, fatty acid synthesis or the synthesis of secondary metabolites from plants, such as polyphenols or polyketides.

The subject matter of the present invention also includes a coryneform bacteria cell for producing polyphenols or polyketides, which has modifications of the aforementioned type according to the invention and in which the catabolic pathway of aromatic components, preferably selected from the group comprising phenylpropanoids and benzoic acid derivatives, is additionally turned off. Coryneform bacteria have their own metabolic pathway for the degradation of phenylpropanoids or benzoic acid derivatives (Kallscheuer et al., 2016; https://doi.org/10.1007/s00253-015-7165-1). This would be counter-productive for the production of polyketides or polyphenols with coryneform bacteria. According to embodiments of the invention, a coryneform bacteria cell is provided for this, allowing and increased provision of malonyl-CoA and further distinguished in that the functionality and/or activity of the enzymes or the expression of the genes coding them and involved in the catabolic pathway of aromatic components are turned off by deletions of the gene clusters cg0344-47 (phdBCDE operon), cg2625-40 (cat, ben and pca), cg 1226 (pobA) and cg0502 (qsuB). These cells according to embodiments of the invention are purposefully modified and have not been produced by random mutagenesis. They are advantageously distinguished by the fact that they are characterized in a genetically precise manner and the said modifications are achieved by deletions. According to embodiments of the invention, these deletions are chromosomally coded. Thus, these cells have exclusively homologous DNA and are non-recombinantly altered. This distinguishes them, in addition to the feature of belonging to the GRAS organisms, advantageously for a microbial production of products, such as, for example, secondary plant metabolites. The coryneform bacteria cell according to embodiments of the invention is also advantageously characterized in that it does not require extrachromosomal DNA, such as plasmids or vectors, for the increased provision of malonyl-CoA and for avoiding the degradation of aromatic components.

The subject matter of the present invention also includes a coryneform bacteria cell which, in addition to the modifications of the aforementioned type according to the invention, comprises the enzymes derived from plants or the genes coding them for the polyphenol or polyketide synthesis. In one variant of the present invention, a coryneform bacteria cell is also comprised that contains the genes derived from plants for polyphenol or polyketide production selected from the group comprising the genes 4cl, sts, chs, chi and pcs.

The coryneform bacteria cell according to embodiments of the invention having the properties according to the invention described above is advantageously distinguished in that it can carry out the synthesis of polyketides from 5 malonyl-CoA units. The synthesis of polyphenols can likewise be carried out with the coryneform bacteria cell of the type described above, wherein supplementation of the corresponding culture medium with a polyphenol precursor, such as p-cumaric acid, promotes the conversion of malonyl-CoA to stilbenes or flavonoids. Starting from glucose as the carbon source, the coryneform bacteria cell according to embodiments of the invention requires the enzymes 3-Deoxy-D-arabinoheptulosonate-7-phosphate synthase and tyrosine ammonium lyase coded by the genes aroH and tal, respectively.

In one embodiment of the present invention, a coryneform bacteria cell is comprised that has genes coding for a feedback-resistant 3-deoxy-D-arabinoheptulosonate-7-phosphate synthase (aroH), preferably from E. coli, and for a tyrosine ammonium lyase (tal), preferably from Flavobacterium johnsoniae.

The enzyme 5,7-dihydroxy-2-methylchromone synthase activity (PCS) is a type Ill polyketide synthase (EC 2.3.1.216, UniProt Q58VP7, (Abe et al., 2005; https://doi.org/10.1021/ja0431206). The Aloe arborescens PCS is encoded by the pcs gene and annotated as EC 2.3.1.216, UniProt Q58VP7. The catalytic activity for the synthesis of noreugenin from five molecules of malonyl-CoA is described as a putative function. According to embodiments of the invention, the pcs gene was synthesized from Aloe arborescens by means of C. glutamicum codon usage and used for the cloning and transformation of coryneform bacteria cells according to the invention. However, only very small traces of noreugenin could be detected with the resulting coryneform bacteria cell. That is, the established enzyme PCS from Aloe arborescens and the pcs gene coding it cannot be confirmed in its annotated function in coryneform bacterial cells. Thus, the annotated 5,7-dihydroxy-2-methylchromone synthase activity (PCS) (EC 2.3.1.216, UniProt Q58VP7) is not suitable for use in coryneform bacteria cells according to embodiments of the invention.

By isolating and providing a nucleic acid sequence according to embodiments of the invention coding for a 5,7-dihydroxy-2-methylchromone synthase (PCS_{Short}), with increased activity in coryneform bacteria, a further structural element is made available with the aid of which plant secondary metabolites can advantageously be produced in coryneform bacteria. The 5,7-dihydroxy-2-methylchromone synthase (PCS_{Short}) according to the invention has an amino acid sequence shortened by 10 N terminal amino acids. The resulting plasmid pMKEx2-pcs_{AsCg}-short can be transformed into each of the C. glutamicum strains described above, wherein the product formation is analyzed after appropriate cultivation and sampling. By way of example, the plasmid is transformed into the C. glutamicum strain DelAro⁴-4c/PcCg-C7-mu/asO. The resulting strain C. glutamicum DelAro⁴-4c/PcCg-C7-mu/asO pMKEx2-pcs_{Aa}cg-short is cultured under standard conditions (CGXII+4% Glucose, 1 mM IPTG, 30° C., 130 RPM, 72 h) and the collected samples analyzed for product formation by means of LC MS (see above). With the plasmid pMKEx2-pcs_{shortAsCg}, a significantly increased functionality and a distinct product formation of noreugenin can be demonstrated under standard conditions. A 5,7-dihydroxy-2-methylchromone synthase variant (PCSₛₕₒᵣₜ) according to embodiments of the invention and the nucleic acid sequence pcsₛₕₒᵣₜ coding it are not known to date.

Subject matter of the present invention also includes a protein having an increased 5,7-dihydroxy-2-methylchromone synthase activity (PCSₛₕₒᵣₜ) in one of the above-described coryneform bacteria cells according to the invention for the synthesis of polyketides in coryneform bacteria, wherein the amino acid sequence has at least 70% identity to the amino acid sequence according to SEQ ID NO. 20 or fragments or alleles thereof. In a variant of the present invention, a 5,7-dihydroxy-2-methylchromone synthase containing an amino acid sequence according to SEQ ID NO. 20 or fragments or alleles thereof is included. According to embodiments of the invention, also a 5,7-dihydroxy-2-methylchromone synthase coded by a nucleic acid sequence containing at least 70% identity to the nucleic acid sequence according to SEQ ID NO. 19 or fragments thereof is comprised. In an embodiment of the present invention, a 5,7-dihydroxy-2-methylchromone synthase coded by a nucleic acid sequence according to SEQ ID NO. 19 or fragments thereof is comprised.

Another embodiment of the present invention comprises a nucleic acid sequence (pcsₛₕₒᵣₜ) coding for a 5,7-dihydroxy-2-methylchromone synthase with increased activity for polyketide production in coryneform bacteria selected from the group comprising:
a) a nucleic acid sequence containing at least 70% identity to the nucleic acid sequence according to SEQ ID NO. 19 or fragments thereof,
b) a nucleic acid sequence which, under stringent conditions, hybridizes with a complementary sequence of a nucleic acid sequence according to SEQ ID NO. 19 or fragments thereof,
c) a nucleic acid sequence according to SEQ. ID NO. 19 or fragments thereof, or
d) a nucleic acid sequence coding for a 5,7-dihydroxy-2-methylchromone synthase (PCSₛₕₒᵣₜ) corresponding to each of the nucleic acids in accordance with a)-c) which is adapted to the codon usage of coryneform bacteria, or
e) that differs from these nucleic acid sequences in accordance with a)-d) by the degeneracy of the genetic code or by function-neutral mutations.

The subject matter of the present invention also includes a coryneform bacteria cell of the kind previously described which has a protein with an increased 5,7-dihydroxy-2-methylchromone synthase activity (PCSₛₕₒᵣₜ) and/or a nucleic acid sequence coding for a 5,7-dihydroxy-2-methylchromone synthase (PCSₛₕₒᵣₜ) with increased activity in coryneform bacteria. In a variant of the present invention, a protein having an increased 5,7-dihydroxy-2-methylchromone synthase activity (PCSₛₕₒᵣₜ) with at least 70% identity to the amino acid sequence according to SEQ ID NO. 20 or fragments or alleles thereof is also comprised. A further variant of the present invention also comprises a protein having an increased 5,7-dihydroxy-2-methylchromone synthase activity (PCSₛₕₒᵣₜ) according to SEQ ID NO. 20.

All genes derived from plants or other heterologous systems, such as aroH, tal and/or the genes for polyphenol synthesis, preferably the stilbene and/or flavonoid synthesis, especially to be mentioned the genes sts, chs, chi or the genes for the polyketide synthesis, preferably pcsₛₕₒᵣₜ, have been adapted and optimized for expression in coryneform bacteria to the bacterial codon usage of these coryneform bacteria, preferably that of Corynebacterium glutamicum. The proportion of heterologous nucleic acid sequences is thereby reduced according to the invention and the expression in coryneform bacteria cells is advantageously supported.

In a variant of the present invention, a coryneform bacteria cell of the aforementioned type is also included in which the plant genes are present under the expression control of an inducible promoter. In a further variant, an IPTG-inducible promoter, preferably the promoter T7, is present according to the invention.

In a variant of the present invention, a coryneform bacteria cell according to the invention is included in which the gene 4cl coding for the 4-coumarate-CoA ligase (4CL) is present under the expression control of an inducible promoter, wherein the inducible promoter and the gene regulatively linked therewith are integrated into the genome of the coryneform bacteria cell, i.e. is present chromosomally coded. In a further variant of the present invention, an IPTG inducible promoter, preferably the promoter T7, is used.

The subject matter of the present invention also includes extrachromosomal systems, such as vectors or plasmids, having the required properties for the expression of the required genes for the synthesis of polyphenols or polyketides. In variants of the present invention, the plasmid- or vector-encoded genes are subject to an inducible promoter, preferably an IPTG-inducible promoter, preferably the promoter T7. The use of an inducible promoter has the advantage according to embodiments of the invention that the expression of the genes required for the secondary metabolites can be controlled in a targeted manner, i.e. can be switched on, depending on the growth or cultivation conditions of the coryneform bacteria cells according to embodiments of the invention. The coryneform bacteria cells according to embodiments of the invention of the type described above can thus first be cultivated for an increased provision of malonyl-CoA, which is then further converted to the desired products after targeted induction of the expression of the required genes.

The subject matter of the present invention also includes a coryneform bacteria cell which comprises genes selected from the group comprising
a) 4cl and sts for the synthesis of polyphenols, preferably stilbenes, particularly preferably resveratrol, or
b) chs and chi for the synthesis of polyphenols, preferably flavonoids, particularly preferably naringenin, or
c) pcsₛₕₒᵣₜ for the synthesis of polyketides, preferably noreugenin,
   under the control of an inducible promoter, preferably an IPTG-inducible promoter, particularly preferably the T7 promoter.

As mentioned herein, the present invention is advantageously characterized in that the genes or regions with which they are regulatorily linked are integrated into the genome of the cells according to embodiments of the invention for increased provision (production) of malonyl-CoA, i.e. are present in chromosomally coded form. This provides degrees of freedom to introduce further heterologous plasmid-coded genes into the cells without overwhelming the cell. The known drawbacks that bacterial cells with more than 2 plasmids cannot be propagated in a stable manner, or the huge drawback that plasmids with more than 2 heterologous genes generally do not produce a satisfactory result with regard to stability or expression, is overcome by the highly advantageous system of a coryneform bacteria cell according to embodiments of the invention. By virtue of its construction, it offers a high degree of freedom of which plant or other heterologous genes can be introduced extrachromosomally into the system in order to thus enable a stable microbial production of plant secondary metabolites starting from malonyl-CoA.

In a variant of the present invention there is a coryneform bacteria cell which has genes selected from the group comprising
a) fasB and/or gltA and/or accBCDI, whose functionality and/or expression is specifically modified for an increased provision of malonyl-CoA, and
b) cg0344-47 (phdBCDE operon), cg2625-40 (cat, ben and pca), cg1226 (pobA) and cg0502 (qsuB), whose functionality for the degradation of aromatic components, preferably from the group comprising phenylpropanoids or benzoic acid derivatives, is turned off, and
c) pcsₛₕₒᵣₜ coding for a protein with an increased 5,7-dihydroxy-2-methylchromone synthase activity (PCSₛₕₒᵣₜ) for the synthesis of polyketides, preferably noreugenin, or
d) optionally aroH and tal for the precursor synthesis of polyphenols starting from glucose, and
e) 4cl and sts for the synthesis of polyphenols, preferably stilbenes, particularly preferably resveratrol, or
f) chs and chi for the synthesis of polyphenols, preferably flavonoids, particularly preferably naringenin.

In variants of a bacteria cell according to the invention, the genes according to the invention or the regulatory regions of a) and b) operatively linked thereto are coded in the genome. The genes or the regulatory regions from c)-f) operatively linked to them are present in plasmid-coded form. According to embodiments of the invention, for the production of polyketides, preferably noreugenin, combinations are conceivable, such as, for example, with variants of fasB (substitution mutants or deletion mutants) and Acg0344-47 {phdBCDE operon) and Acg2625-40 {cat, ben and pca) and Acg1226 {pobA) and Acg0502 {qsuB) and pcsₛₕₒᵣₜ; with gtlA and Acg0344-47 {phdBCDE operon) and Acg2625-40 {cat, ben and pca) and Acg1226 {pobA) and Acg0502 {qsuB) and pcsₛₕₒᵣₜ; with gtlA and accBCDI and Acg0344-47 {phdBCDE operon) and Acg2625-40 {cat, ben and pca) and Acg1226 {pobA) and Acg0502 {qsuB) and pcsₛₕₒᵣₜ; with variants of fasB (substitution mutants or deletion mutants) and gtlA and accBCDI and Acg0344-47 {phdBCDE operon) and Acg2625-40 {cat, ben and pca) and Acg1226 {pobA) and Acg0502 {qsuB) and pcsₛₕₒᵣₜ.

Combinations are conceivable for the production of polyphenols, preferably stilbenes, more preferably resveratrol, such as, for example, with variants of fasB (substitution mutants or deletion mutants) and Acg0344-47 (phdBCDE operon) and Acg2625-40 (cat, ben and pca) and Acg1226 (poM) and Acg0502 (qsuB) and aroH and tal and 4cl and sts; with gtlA and Acg0344-47 {phdBCDE operon) and Acg2625-40 {cat, ben and pca) and Acg1226 {pobA) and Acg0502 {qsuB) and aroH and tal and 4cl and sts; with gtlA and accBCDI and Acg0344-47 {phdBCDE operon) and Acg2625-40 {cat, ben and pca) and Acg1226 {pobA) and Acg0502 {qsuB) and aroH and tal and 4cl and sts; with variants of fasB (substitution mutants or deletion mutants) and gtlA and accBCDI and Acg0344-47 {phdBCDE operon) and Acg2625-40 {cat, ben and pca) and Acg1226 {pobA) and Acg0502 {qsuB) and aroH and tal and 4cl and sts. These variants mentioned above allow the production of the polyphenols starting from glucose on account of the expression of the genes aroH and tal. However, the genes aroH and tal are not required for a cultivation of the coryneform bacteria cell according to embodiments of the invention supplemented with the precursor p-cumaric acid. Variants according to the invention of the aforementioned coryneform bacteria cell then do not have the genes aroH and tal.

For the preparation of polyphenols, preferably flavonoids, more preferably naringenin, combinations are conceivable, such as, for example, with variants of fasB (substitution mutants or deletion mutants) and Acg0344-47 (phdBCDE operon) and Acg2625-40 (cat, ben and pca) and Acg1226 (pobA) and Acg0502 (qsuB) and aroH and tal and chs and chi; with gtlA and Acg0344-47 {phdBCDE operon) and Acg2625-40 (cat, ben and pca) and Acg1226 (pobA) and Acg0502 (qsuB) and aroH and tal and chs and chi; with gtlA and accBCDI and Acg0344-47 (phdBCDE operon) and Acg2625-40 (cat, ben and pca) and Acg1226 (pobA) and Acg0502 (qsuB) and aroH and tal and chs and chi; with variants of fasB (substitution mutants or deletion mutants) and gtlA and accBCDI and Acg0344-47 (phdBCDE operon) and Acg2625-40 (cat, ben and pca) and Acg1226 (pobA) and Acg0502 (qsuB) and aroH and tal and chs and chi. These variants mentioned above allow the production of the polyphenols starting from glucose on account of the expression of the genes aroH and tal. However, the genes aroH and tal are not required for a cultivation of the coryneform bacteria cell according to the invention supplemented with the precursor p-cumaric acid. Variants of the aforementioned coryneform bacteria cell according to the invention then optionally do not have the genes aroH and tal.

In further variants of the present invention there is a coryneform bacteria cell of the aforementioned type with the aforementioned variations in gene combinations comprising genes selected from the group comprising
a) fasB gene according to a nucleic acid sequence selected from the group comprising SEQ ID NO. 1, 3, 5, 7, and 9 or fragments thereof, coding for fatty acid synthases FasB selected from the group comprising SEQ ID NO. 2, 4, 6, 8, and 10, or fragments or alleles thereof, and/or gltA gene with operatively linked promoter region according to SEQ ID NO. 11 and/or accBCDI gene clusters with operatively linked fasO binding sites selected from the group comprising SEQ ID NO. 13 and 15, whose functionality and/or expression is specifically modified for an increased provision of malonyl-CoA, and
b) cg0344-47 (phdBCDE operon), cg2625-40 (cat, ben and pca), cg1226 (pobA) and cg0502 (qsuB), whose functionality for the degradation of aromatic components, preferably from the group comprising phenylpropanoids or benzoic acid derivatives, is turned off, and
c) pcsₛₕₒᵣₜ according to SEQ ID NO. 19 coding for a protein with an increased 5,7-dihydroxy-2-methylchromone synthase activity (PCSₛₕₒᵣₜ) according to SEQ ID NO: 20 or fragments or alleles thereof for the synthesis of polyketides, preferably noreugenin, or
d) optionally aroH according to SEQ ID NO. 30 or fragments or alleles thereof, and tal according to SEQ ID NO: 32 or fragments or alleles thereof, for the precursor synthesis of polyphenols starting from glucose, and
e) 4cl according to SEQ ID NO. 22 or fragments or alleles thereof, and sts according to SEQ ID 24 or fragments or alleles thereof, for the synthesis of polyphenols, preferably stilbenes, particularly preferably resveratrol, or
f) chs according to SEQ ID NO. 26 or fragments or alleles thereof, and chi according to SEQ ID NO. 28 or fragments or alleles thereof, for the synthesis of polyphenols, preferably flavonoids, particularly preferably naringenin.

The listed variants are the subject matter of the invention without the invention being limited thereby. This description serves for a better understanding of the present invention.

Subject matter of the present invention further includes a method for the microbial preparation of malonyl-CoA in coryneform bacteria comprising the steps of:
a) providing a solution containing water and a C6 carbon source;
b) microbial conversion of the C6 carbon source in a solution according to step a) to malonyl-CoA in the presence of a coryneform bacteria cell according to the invention in which the regulation and/or expression of the genes selected from the group comprising fasB, gtlA, accBC and accD1 and/or the functionality of the enzymes encoded by them is specifically modified.

According to the invention, “solution” is equivalent in meaning to “medium,” “culture medium,” “culture broth,” or “culture solution”. Within the meaning of the present invention, “microbial” is equivalent in meaning to “biotechnological” or “fermentative.” According to the invention, “reaction” is equivalent in meaning to “metabolization” or “cultivation”. According to the invention, “conditioning” is equivalent in meaning to “separation,” “concentration,” or “purification”.

The culture medium to be used should adequately satisfy the requirements of the respective microorganisms. Descriptions of culture media of various microorganisms are contained in the handbook “Manual of Methods for General Bacteriology” of the American Society for Bacteriology (Washington D.C., USA, 1981). Besides glucose as starting substrate for malonyl-CoA provision, sugar and carbohydrates, such as glucose, sucrose, lactose, fructose, maltose, molasses, starch and cellulose, oils and fats, such as soy oil, sunflower oil, peanut oil, and coconut oil, fatty acids, such as palmitic acid, stearic acid, and linoleic acid, alcohols, such as glycerol and ethanol, and organic acids, such as acetic acid, can be used as carbon source. These substances can be used individually or as a mixture. The nitrogen source used may be organic, nitrogen-containing compounds, such as peptones, yeast extract, meat extract, malt extract, maize steeping liquor, soybean meal, and urea or inorganic compounds, such as ammonium sulfate, ammonium chloride, ammonium phosphate, ammonium carbonate, and ammonium nitrate. The nitrogen sources can be used individually or as a mixture. Potassium dihydrogen phosphate or dipotassium hydrogen phosphate or the corresponding sodium-containing salts can be used as phosphorus source. The culture medium should furthermore contain salts of metals, such as magnesium sulfate or iron sulfate, which are necessary for growth. Ultimately, it is possible to use essential growth substances, such as amino acids and vitamins, in addition to the aforementioned substances. Said starting materials can be added to the culture in the form of a single batch or fed in a suitable manner during the cultivation. For the pH control of the culture, basic compounds such as sodium hydroxide, potassium hydroxide, ammonia or acidic compounds such as hydrochloric acid, phosphoric acid or sulfuric acid are used in a suitable manner. Antifoam agents, such as fatty acid polyglycol esters, can be used to control foam development. Suitable selective substances, such as antibiotics, can be added to the medium in order to maintain the stability of plasmids. In order to maintain aerobic conditions, oxygen or oxygen-containing gas mixtures, such as air, are introduced into the culture. The temperature of the culture is normally from about 20° C. to about 45° C., and preferably from about 25° C. to about 40° C.

The present invention relates to a method in which cultivation takes place discontinuously or continuously, preferably in batch, fed batch, repeated fed batch or continuous mode.

In a variant of the method according to the invention for the increased provision of malonyl-CoA, the microbial conversion of the C6 carbon source takes place in a coryneform bacteria according to embodiments of the invention containing one of the variants of fasB described according to embodiments of the invention, in which the fatty acid synthase FasB is reduced or turned off and/or the gene fasB coding for fatty acid synthase is purposefully mutated, preferably by one or more nucleotide substitutions, or partially or completely deleted.

In a variant of the method according to the invention for the increased provision of malonyl-CoA, the microbial conversion of the C6 carbon source takes place in a coryneform bacteria cell according to embodiments of the invention containing a gene gltA coding for citrate synthase according to embodiments of the invention, said gene being reduced in its expression by mutation, preferably multiple nucleotide substitutions, of the operatively linked promoter.

In a variant of the method according to the invention for the increased provision of malonyl-CoA, the microbial conversion of the C6 carbon source in a coryneform bacteria cell according to embodiments of the invention containing the accBC and accD1 genes according to embodiments of the invention, for which the functionality of the operator binding sites (fasO) for the regulator FasR in the promoter areas of the gene accBC and accD1 coding for the acetyl-CoA carboxylase subunits, preferably by one or more nucleotide substitutions, is reduced or turned off and the expression of the genes accBC and accD1 coding for the acetyl-CoA carboxylase subunits is derepressed, preferably increased.

In a further variant of the method according to the invention for the increased provision of malonyl-CoA, the microbial conversion of the C6 carbon source takes place in a coryneform bacteria cell according to embodiments of the invention which has a combination of reduced expression and/or activity of the citrate synthase (CS) and deregulated, increased expression and/or activity of the acetyl-CoA carboxylase subunits (AccBC and AccD1).

In a further variant of the method according to the invention for the increased provision of malonyl-CoA, the microbial conversion of the C6 carbon source takes place in a coryneform bacteria cell according to embodiments of the invention which has a combination of reduced expression and/or activity of the citrate synthase (CS) and deregulated, increased expression and/or activity of the acetyl-CoA carboxylase subunits (AccBC and AccD1) and reduced or eliminated functionality of the fatty acid synthase FasB.

In a further variant of the method according to the invention for the increased provision of malonyl-CoA, the microbial conversion of the C6 carbon source takes place in a coryneform bacterial cell of the genus Corynebacterium or Brevibacterium according to embodiments of the invention. In a further variant of the method according to the invention for the increased provision of malonyl-CoA, the microbial conversion of the C6 carbon source takes place in a coryneform bacterial cell according to embodiments of the invention, selected from the group comprising Corynebacterium glutamicum, particularly preferred Corynebacterium glutamicum ATCC 13032, Corynebacterium acetoglutamicum, Corynebacterium thermoaminogenes, Brevibacterium flavum, Brevibacterium lactofermentum or Brevibacterium divaricatum. According to embodiments of the invention, a variant of the method according to the invention for the increased provision of malonyl-CoA is carried out by microbial conversion of the C6 carbon source in a coryneform bacteria cell according to the invention, such as Corynebacterium glutamicum ATCC13032 or deliberately modified derivatives or archetypes thereof, such as, for example, Corynebacterium glutamicum ATCC13032, in which in addition the catabolic pathway of aromatic components, preferably selected from the group comprising phenylpropanoids and benzoic acid derivatives, is turned off.

The subject matter of the present invention furthermore includes a method for the microbial preparation of polyphenols or polyketides in coryneform bacteria, comprising the steps of:
a) providing a solution containing water and a C6 carbon source,
b) microbial conversion of the C6 carbon source in a solution according to step a) to polyphenols or polyketides in the presence of a coryneform bacteria cell according to the invention, wherein malonyl-CoA is first provided at an elevated concentration as intermediate and further reacted for microbial synthesis of polyphenols or polyketides;
c) induction of the expression of heterologous or plant genes under the control of an inducible promoter by addition of a suitable inducer in step b),
d) optionally the conditioning of the desired product.

In a variant of the method according to the invention, a coryneform bacteria cell is used which has genes selected from the group comprising:
a) fasB and/or gltA and/or accBCDI, whose functionality and/or expression is specifically modified for an increased provision of malonyl-CoA, and
b) cg0344-47 (phdBCDE operon), cg2625-40 (cat, ben and pca), cg1226 (pobA) and cg0502 (qsuB), whose functionality for the degradation of aromatic components, preferably from the group comprising phenylpropanoids or benzoic acid derivatives, is turned off, and
c) pcsₛₕₒᵣₜ coding for a protein with an increased 5,7-dihydroxy-2-methylchromone synthase activity (PCSₛₕₒᵣₜ) for the synthesis of polyketides, preferably noreugenin, or
d) aroH and tal for the precursors synthesis of polyphenols starting from glucose, and
e) 4cl and sts for the synthesis of polyphenols, preferably stilbenes, particularly preferably resveratrol, or
f) chs and chi for the synthesis of polyphenols, preferably flavonoids, particularly preferably naringenin.

According to embodiments of the invention, combinations are conceivable here for the production of polyketides, preferably noreugenin, such as, for example, with variants of fasB (substitution mutants or deletion mutants) and Acg0344-47 {phdBCDE operon) and Acg2625-40 {cat, ben and pca) and Acg1226 {pobA) and Acg0502 {qsuB) and pcsₛₕₒᵣₜ; or with gtlA and Acg0344-47 {phdBCDE operon) and Acg2625-40 {cat, ben and pca) and Acg1226 {pobA) and Acg0502 {qsuB) and pcsₛₕₒᵣₜ; or with gtlA and accBCDI and Acg0344-47 {phdBCDE operon) and Acg2625-40 {cat, ben and pca) and Acg1226 {pobA) and Acg0502 {qsuB) and pcs_{Short}; or with variants of fasB (substitution mutants or deletion mutants) and gtlA and accBCDI and Acg0344-47 {phdBCDE operon) and Acg2625-40 {cat, ben and pca) and Acg1226 {pobA) and Acg0502 {qsuB) and pcsₛₕₒᵣₜ.

According to embodiments of the invention, combinations are conceivable for the production of polyphenols, preferably stilbenes, more preferably resveratrol, such as, for example, with variants of fasB (substitution mutants or deletion mutants) and Acg0344-47 (phdBCDE operon) and Acg2625-40 (cat, ben and pca) and Acg1226 {pobA) and Acg0502 {qsuB) and aroH and tal and 4cl and sts; with gtlA and Acg0344-47 {phdBCDE operon) and Acg2625-40 {cat, ben and pca) and Acg1226 {pobA) and Acg0502 {qsuB) and aroH and tal and 4cl and sts; with gtlA and accBCDI and Acg0344-47 {phdBCDE operon) and Acg2625-40 {cat, ben and pca) and Acg1226 {pobA) and Acg0502 {qsuB) and aroH and tal and 4cl and sts; with variants of fasB (substitution mutants or deletion mutants) and gtlA and accBCDI and Acg0344-47 {phdBCDE operon) and Acg2625-40 {cat, ben and pca) and Acg1226 (pobA) and Acg0502 (qsuB) and aroH and tal and 4cl and sts. These variants mentioned above allow the production of the polyphenols starting from glucose on account of the expression of the genes aroH and tal. However, the genes aroH and tal are not required for a cultivation of the coryneform bacteria cell according to embodiments of the invention supplemented with the precursor p-cumaric acid. Variants according to the invention of the aforementioned coryneform bacteria cell then do not have the genes aroH and tal or the expression of these genes is not induced.

According to embodiments of the invention, for the preparation of polyphenols, preferably flavonoids, more preferably naringenin, combinations are conceivable, such as, for example, with variants of fasB (substitution mutants or deletion mutants) and Acg0344-47 (phdBCDE operon) and Acg2625-40 (cat, ben and pca) and Acg1226 (pobA) and Acg0502 (qsuB) and aroH and tal and chs and chi; with gtlA and Acg0344-47 (phdBCDE operon) and Acg2625-40 (cat, ben and pca) and Acg1226 (pobA) and Acg0502 (qsuB) and aroH and tal and chs and chi; with gtlA and accBCDI and Acg0344-47 (phdBCDE operon) and Acg2625-40 (cat, ben and pca) and Acg1226 (pobA) and Acg0502 (qsuB) and aroH and tal and chs and chi; with variants of fasB (substitution mutants or deletion mutants) and gtlA and accBCDI and Acg0344-47 (phdBCDE operon) and Acg2625-40 (cat, ben and pca) and Acg1226 (pobA) and Acg0502 (qsuB) and aroH and tal and chs and chi. These variants mentioned above allow the production of the polyphenols starting from glucose on account of the expression of the genes aroH and tal. However, the genes aroH and tal are not required for a cultivation of the coryneform bacteria cell according to embodiments of the invention supplemented with the precursor p-cumaric acid. Variants of the aforementioned coryneform bacteria cell according to the invention then optionally do not have the genes aroH and tal.

In a variant of the method according to the invention for polyphenol production, the solution in step b) is supplemented with the polyphenol precursor, preferably p-cumaric acid.

Here, supplementation with p-cumaric acid in a concentration of 1-10 mM, preferably 2-8 mM, particularly preferably 3-7 mM, very particularly preferably 5-6 mM and in particular 5 mM and all conceivable intermediates is suitable.

According to embodiments of the invention, “conditioning” is equivalent in meaning to “separation,” “extraction”, “concentration”, or “purification”. Product preparation is optional in the method for the preparation of polyketides and polyphenols according to the invention, since the advantageous, targeted stem construction of coryneform bacteria according to the invention achieves the production of only one secondary metabolite, such as, for example, resveratrol or naringenin or noreugenin. This advantageously does not require the separation of several different products, such as, for example, resveratrol and naringenin, from the culture solution. This is another advantage of embodiments of the present invention. The method according to embodiments of the invention is advantageously distinguished in that it is independent of the addition of inhibitors of fatty acid synthesis, for example cerulenin. Further extraction, preparation of the cells, cell extracts or cell supernatants are known to the person skilled in the art and can take place in a known manner.

In variants of the method according to the invention, the cultivation takes place in a discontinuous or continuous, preferably batch, fed batch, repeated fed batch or continuous mode. The procedures required for carrying out such cultivation methods are known to the person skilled in the art.

The subject matter of the present invention also includes the use of a coryneform bacteria cell of the above-described type and/or one or more proteins according to embodiments of the invention and/or one or more nucleotide sequences according to embodiments of the invention for the increased provision of malonyl-CoA in coryneform bacteria.

The subject matter of the present invention also includes the use of a coryneform bacteria cell according to embodiments of the invention and/or one or more proteins according to the invention and/or one or more nucleotide sequences according to embodiments of the invention for polyketide or polyphenol production, preferably for producing noreugenin or for producing stilbenes, particularly preferably resveratrol, or for producing flavonoids, particularly preferably naringenin.

The subject matter of the present invention also includes a composition containing secondary metabolites selected from the group of polyphenols and polyketides, preferably stilbenes, flavonoids or polyketides, particularly preferably resveratrol, naringenin and/or noreugenin, produced with a coryneform bacteria cell according to embodiments of the invention and/or one or more proteins according to embodiments of the invention and/or one or more nucleotide sequences according to embodiments of the invention and/or a method of the above-described type according to embodiments of the invention.

The subject matter of the present invention further includes the use of resveratrol, naringenin and/or noreugenin produced with a coryneform bacteria cell according to embodiments of the invention and/or according to a method according to embodiments of the invention and/or the use of a composition of the above-described type for producing pharmaceuticals, foodstuffs, feedstuffs and/or for use in plant physiology. The composition according to embodiments of the invention may comprise further substances which are advantageous in the preparation of the desired products. A selection is known to the person skilled in the art from the prior art.

### Examples

The present invention is explained in more detail by the following examples, which, however, are not limiting:

Alteration of the Regulatory Binding Site in the Promoter Region of the Citrate Synthase CS by Nucleotide Substitutions for the Integration into the Genome of Coryneform Bacteria Cells
Cloning pK19mobsacB-PgltA::PdapA-C7

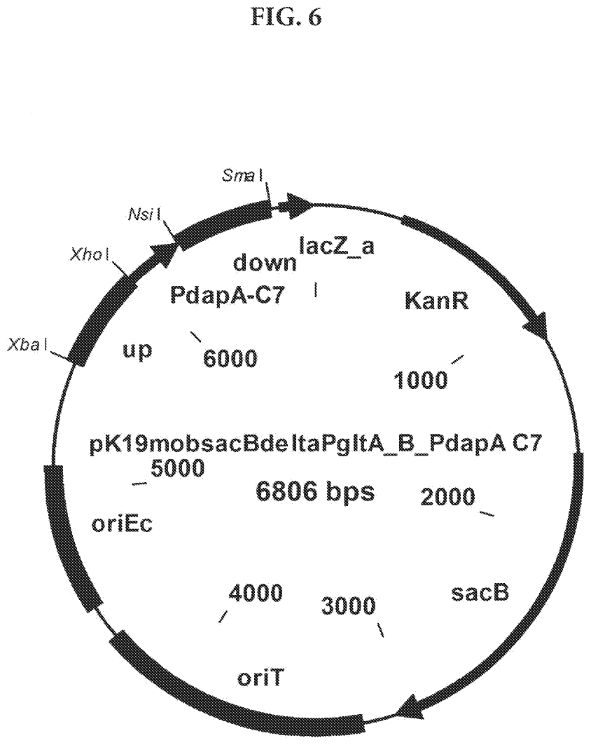

The plasmid pK19mobsacB-A540 was constructed first to then construct the plasmid pK19mobsacB-PgltA::PdapA-C7 (FIG. 6). Here, the flanking regions were chosen such that a 540 base pair chromosomal fragment bearing the native gltA promoter region with the two transcription start and operator sequences can be deleted. A 20 base pair linker having the interfaces Nsi\ and Xho\ was inserted between the two flanks up and down. The C7 variant of the dapA promoter was subsequently subcloned via these interfaces.

For the cloning of pK19mobsacB-A540, the upstream fragment up was amplified with the primer pair PgltA-up-s/PgltA-up-as, the downstream flank was amplified with the primer pair PgltA-down-s/PgltA-down-as. The check of the generated DNA fragments for the expected base pair size was performed by means of gel electrophoretic analysis on a 1% agarose gel. The nucleotide sequences of the inner primers (PgltA-up-as/PgltA-down-s) were selected such that the two amplified fragments up and down contain respective complementary overhangs (including the A/s/l/X/jol linker described). In a second PCR (without addition of DNA primers), the purified fragments attach via the complementary sequences and serve as both primers and templates for each other (overlap extension PCR). The A540 fragment thus generated was amplified in a final PCR with the two exterior (facing away from the gene) primers from the first PCR (PgltA-up-s/PgltA-down-as). After electrophoretic separation on a 1% TAE agarose gel, the final mutation fragment was isolated from the gel with the NucleoSpin® Gel and PCR Clean-up Kit (Macherey-Nagel, Düren) according to the accompanying protocol. For the construction of pk19mobsacB-A540, both the generated A540 fragment and the pK19mobsacB empty vector were digested with FastDigest variants (Thermo Fisher Scientific) of restriction enzymes Xba\ and Sma\. The restriction assays of said fragments were purified with the NucleoSpin Gel and PCR Clean-up-KA (Macherey-Nagel, Düren). For the ligation of the hydrolyzed DNA fragments by means of the Rapid DNA Ligation Kit (Thermo Fisher Scientific), the deletion fragment was used in threefold molar excess relative to the linearized vector backbone pK19mobsacB. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL of LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/mL) and incubated overnight at 37° C. The correct assembly of pk19mobsacB-A540 in the grown transformants was checked by means of colony PCR. The 2× DreamTaq Green PCR Master Mix (ThermoFisher Scientific inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct ligation of the fragments used, forms a PCR product of a specific size which was checked by gel electrophoresis. Clones whose PCR product indicates a correct assembly of pK19mobsacB-A540 were grown overnight in LB medium with kanamycin (50 pg/mL) for isolation of the plasmids. The plasmids were then isolated with the NucleoSpin Plasmid (NoLid)-KA (Macherey-Nagel, Düren) and sequenced with said amplification and colony PCR primers.

For the construction of pk19mobsacB-PgltA::PdapA-C7, the C7 variant of the dapA promoter was amplified with the primer pair PdapA-s/PdapA-as and checked for the expected base pair size by means of gel electrophoretic analysis on a 1% agarose gel. The generated fragment was purified with the NucleoSpin® Gel and PCR Clean-up Kit (Macherey-Nagel, Düren) according to the accompanying protocol. For the construction of pk19mobsacB-PgltA::PdapA-C7, both the generated PdapA fragment and the target vector pk19mobsacB-A540 were digested with the FastDigest variants (Thermo Fisher Scientific) of the restriction enzymes Xho\ and Nsi\. The restriction assays of said fragments were purified with the NucleoSpin Gel and PCR Clean-up Kit (Macherey Nagel, Düren). For ligation of the hydrolyzed DNA fragments by means of the Rapid DNA Ligation-Kit (Thermo Fisher Scientific), the PdapA fragment was used in threefold molar excess over the linearized vector backbone pk19mobsacB-A540. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/mL) and incubated overnight at 37° C. The correct assembly of pk19mobsacB-PgltA::PdapA-C7 in the grown transformants was checked by means of colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct ligation of the fragments used, forms a PCR product of a specific size which was checked by gel electrophoresis. Clones whose PCR product indicates a correct assembly of pk19mobsacB-PgltA::PdapA-C7 were grown overnight in LB medium with kanamycin (50 pg/mL) for isolation of the plasmids. The plasmids were then isolated with the NucleoSpin Plasmid (NoLid)-KW (Macherey-Nagel, Düren) and sequenced with said amplification and colony PCR primers.

An aliquot of electrocompetent C. glutamicum cells was transformed with the described protocol with pk19mobsacB-PgltA::PdapA-C7 and spread on BHIS Kan¹⁵ plates. Since the pK19mobsacB plasmid cannot replicate in C. glutamicum, the subsequent selection of the mediated kanamycin resistance could be expected to be formed only if the mutation plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the mutation plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the mutation plasmid into their genome via homologous recombination are resistant to kanamycin and sensitive to sucrose.

The excision of pK19mobsacB took place in a second recombination event via the now doubly present DNA regions in which the codon to be mutated from the chromosome was eventually exchanged for the introduced mutation fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired mutation. For the detection of the successful exchange in the clones obtained after excision, the corresponding genomic region was amplified by colony PCR (primer pair chk-PgltA-s/chk-PgltA-as) and checked for the expected fragment formation by gel electrophoresis. PCR products indicating a promoter exchange were purified with the NucleoSpin Gel and PCR Clean-up-Kit (Macherey-Nagel, Düren) and sequenced with primers chk-PgltA-s and chk-PgltA-as to verify the exchange.

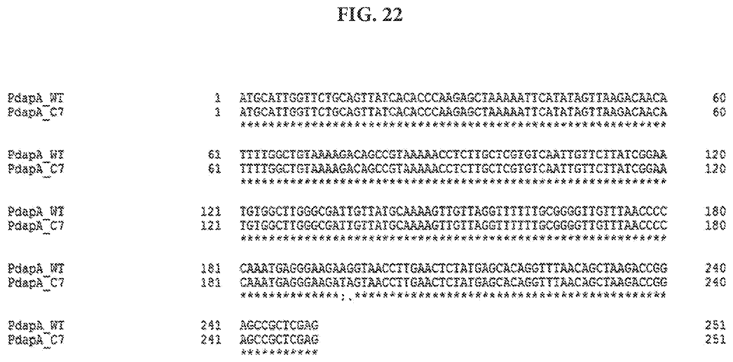

The promoter region PgltA::PdapA-C7 according to an embodiment of the invention furthermore has, in addition to a replacement of the promoter region of gtlA with dapA, additional nucleotide substitutions at positions 95 (a->t) and 96 (g->a) upstream of the start codon ATG (FIG. 22).

Primers used
PgltA-up-s: TGCTCT AGAGCAT GAACTGGGACTT GAAG
PgltA-up-as:
TATG CATGTTT CTCGAGT GGGCCGAAC AAAT ATGTTT GAAAG G
PgltA-down-s:
CCCACTCGAGAAACATGCATAGCGTTTTCAATAGTTCGGTGTC
PgltA-down-as:
CCCCCCGGGGGGCCTAGGGAAAGGATGATCTCGTAGCC
PdapA-s:
CCAATGCATTGGTTCTGCAGTTATCACACCCAAGAGCTAAAAATTCA
PdapA-as:
CCGCTCGAGCGGCTCCGGTCTTAGCTGTTAAACCT
chk-PgltA-s:
ATGAGTCCGAAGGTTGCTGCAT
chk-PgltA-as:
TCGAGTGGGTTCAGCTGGTCC
univ:
CGCCAGGGTTTTCCCAGTCACGAC
rsp:
CA CA GGAAAC AGCTAT GACCATG

Alteration of the Regulatory Binding Site (Operator: fasO) for the FasR Regulator Protein in the Promoter Region of the Acetyl Carboxylases AccBCDI by Nucleotide Substitutions for Integration into the Genome of Coryneform Bacteria Cells
Construction pK19mobsacB-mufasO-accBC and pK19mobsacB-mufasO-accD1

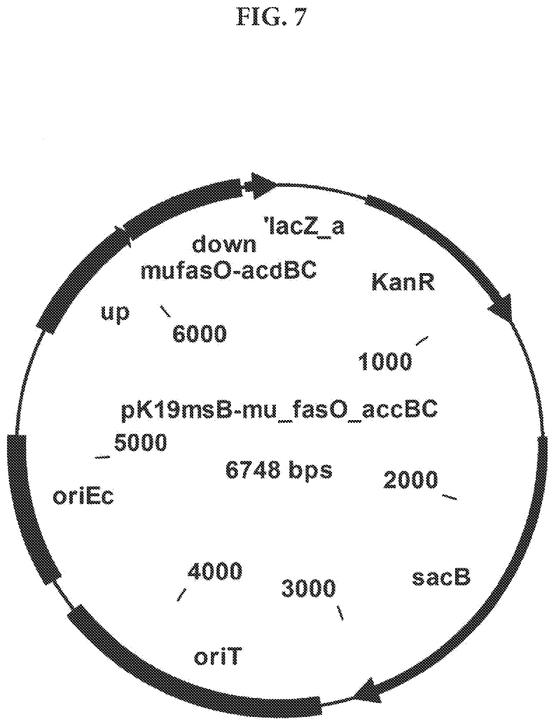

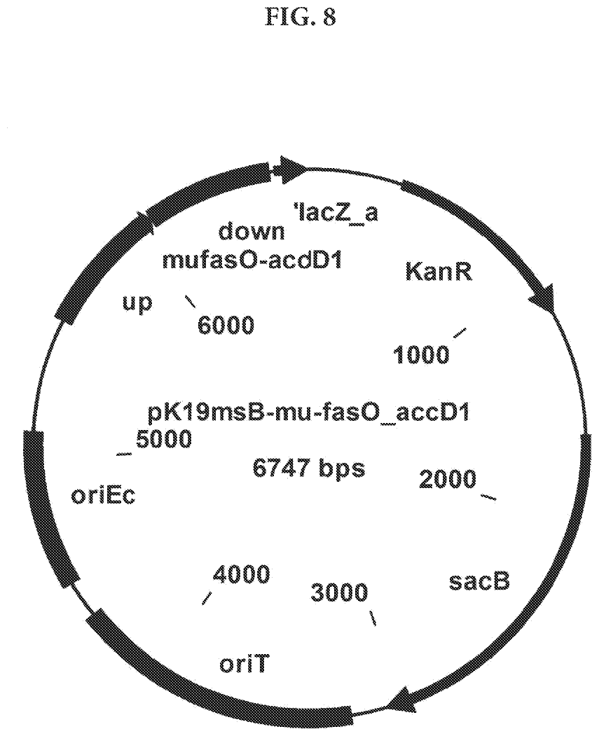

For the construction of the plasmids pK19mobsacB-mufasO-accBC (FIG. 7) and pK19mobsacB-mufasO-accD1 (FIG. 8) for the mutation of the respective/asO binding site of the genes accBC and accD1 in C. glutamicum, the flanking fragments required for the homologous recombination event were amplified by PCR starting from isolated genomic C. glutamicum DNA.

For the generation of the upstream fragment, the primer pair mu-accXX-up-s/mu-accXX-up-as was used, the downstream flank was amplified with the primer pair mu-accXX-down-s/mu-accXX-down-as. The coding XX here represents one of the two acc gene variants (accBC or accD1) respectively. The nucleotide sequences of the inner primers (facing the gene to be deleted) (fasB-(cg2743)-up-as/fasB-(cg2743)-down-s) were selected such that the two amplified fragments up and down contain mutually complementary overhangs which are a prerequisite for the later Gibson assembly. Furthermore, the planned mutations within the respective fasO binding site are introduced via these primers. The verification of the generated DNA fragments for the expected base pair size was carried out by means of gel electrophoresis analysis on a 1% agarose gel and subsequently purified with the NucleoSpin® Gel and PCR Clean-up Kit (Macherey-Nagel, Düren) according to the accompanying protocol. For the construction of the mutation plasmids, the empty vector pK19-mobsacB was linearized with the FastDigest variant (Thermo Fisher Scientific) of the restriction enzyme EcoRI. The restriction assay was purified with the NucleoSpin Gel and PCR Clean-up Kit (Macherey Nagel, Düren). For the assembly of the DNA fragments by means of Gibson Assembly (Gibson et al., 2009a), the amplified fragments were used in threefold molar excess over the linearized vector backbone pK19mobsacB. The DNA fragments were provided with a prepared Gibson Assembly Master Mix which, in addition to an isothermal reaction buffer, contains the enzymes required for assembly (T5 exonuclease, phusion DNA polymerase and Taq DNA ligase). The assembly of the fragments is carried out at 50° C. for 60 minutes in a thermal cycler. After the assembly of the fragments, the total batch volume was used for transformation of chemically competent E. coli DH5a cells by heat shock at 42° C. for 90 seconds. Following the heat shock, the cells were regenerated on ice for 90 seconds before being provided with 800 μL LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/mL) and incubated overnight at 37° C. The correct assembly of the mutation plasmids in the grown transformants was verified by colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct assembly of the fragments used, forms a PCR product of a specific size which was checked by gel electrophoresis. Clones whose PCR product indicates a correct assembly of the mutation plasmids pK19mobsacB-mufasO-accBC and/or pK19mobsacB-mufasO-accD1 were grown overnight in LB medium with kanamycin (50 pg/mL) for isolation of the plasmids. The plasmids were then isolated with the NucleoSpin Plasmid (NoLid) Kit (Macherey-Nagel, Düren) and sequenced with said amplification and colony PCR primers.

An aliquot of electrocompetent C. glutamicum cells was transformed with the described protocol with the respective mutation plasmid and spread on BHIS Kan¹⁵ plates. Since the pK19mobsacB plasmid cannot replicate in C. glutamicum, the subsequent selection of the mediated kanamycin resistance could be expected to be formed only if the mutation plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the mutation plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the mutation plasmid into their genome via homologous recombination are resistant to kanamycin and sensitive to sucrose.

The excision of pK19mobsacB took place in a second recombination event via the now doubly present DNA regions in which the codon to be mutated from the chromosome was eventually exchanged for the introduced mutation fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired mutation. For the detection of the successful mutation in the clones obtained after excision, the corresponding genomic region was amplified by colony PCR (primer pair chk_accXX_s/chk_accXX_as). The PCR products were purified with the NucleoSpin Gel and PCR Clean-up-Kit (Macherey-Nagel, Düren) and sequenced to verify the mutation with the primers chk_accXX_s/chk_accXX_as.

According to embodiments of the invention, nucleotide substitutions are thus present at the fasO binding site upstream of accBC at positions 11-13 (tga->gtc) and 20-22 (cct->aag). Nucleotide substitutions are present at positions 20-24 (cctca->gtacg) in the fasO binding site upstream of accD1. In a variant of the present invention, the fasO binding sites according to the invention have a nucleic acid sequence upstream of the genes accBD and accD1, according to SEQ ID NO: 13 and 15, respectively.

Primers used
univ:
CGCCAGGGTTTTCCCAGTCACGAC
rsp:
CACAGGAAACAGCTAT GACCAT G
mufasO-accBC
mu-accBC-up-s:
ATCCCCGGGTACCGAGCTCGAACCAGCGCGCGTTCGTG
mu-accBC-up-as:
TTACGACTATTCTGGGGGAATTCTTCTGTTTTAGGCAGGAAATA
TGGCTTATG
mu-accBC-down-s:
AGAAGAATTCCCCCAGAATAGTCGTAAGTAAGCATATCTGGTT
GAGTTCTTCGGGGTTG
mu-accBC-down-as:
TTGTAAAACGACGGCCAGTGGCCTTGGCGGTATCTGCG
chk-accBC-s:
GTTCGGCCACTCCGATGTCCGCCTG
chk-accBC-as:
GCCTTGATGGCGATTGGGAGACC
mufasO-accD1
mu-accD1-up-s:
ATCCCCGGGTACCGAGCTCGTCATTCAACGCATCCATGACAGC
mu-accD1-up-as:
CTAATGGTCATGTTTTGAAATCGTAGCGGTAGGCGGGG
mu-accD1-down-s:
ACCGCTACGATTT CAAAACAT GACCATTAGT AGCCCTTT G ATT
GACGT CGCCAACCTT C
mu-accD1-down-as:
TTGTAAAACGACGGCCAGTGCGCCAGAAGCCTGAATGTTTTG
chk-accD1-s:
GGCTGATATTAGTGCCCCAACCGATGAC
chk-accD1-as:
GATCACGTCTGGGCCGGTAACGAAC

Deletion of the Gene fasB for the Elimination of the Functionality of the Fatty Acid Synthase FasB for Integration into the Genome of Coryneform Bacteria Cells
Construction pK19mobsacB-AfasB

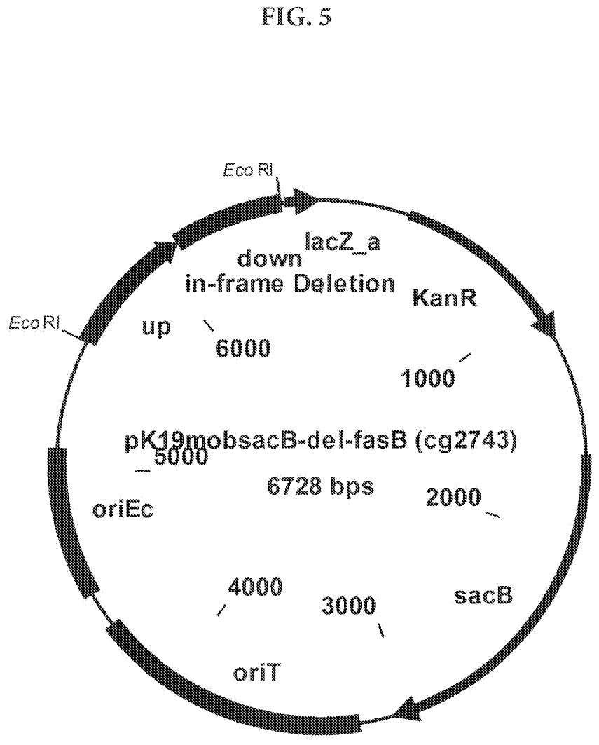

For the construction of the plasmid pK19mobsacB-AfasB (FIG. 5) for the deletion of the gene fasB in C. glutamicum, the flanking fragments required for the homologous recombination event were amplified by PCR starting from isolated genomic C. glutamicum DNA.

For the generation of the upstream fragment, the primer pair fasB-(cg2743)-up-s/fasB-(cg2743)-up-as was used, the downstream flank was amplified with the primer pair fasB-(cg2743)-down-s/fasB-(cg2743)-down-as. The nucleotide sequences of the inner primers (facing the gene to be deleted) (fasB-(cg2743)-up-as/fasB-(cg2743)-down-s) were selected such that the two amplified fragments up and down contain mutually complementary overhangs which are a prerequisite for the later Gibson assembly. The verification of the generated DNA fragments for the expected base pair size was carried out by means of gel electrophoresis analysis on a 1% agarose gel and subsequently purified with the NucleoSpin® Gel and PCR Clean-up Kit (Macherey-Nagel, Düren) according to the accompanying protocol. For the construction of the deletion plasmid, the empty vector pK19-mobsacB was linearized with the Fast-Digest variant (Thermo Fisher Scientific) of the restriction enzyme EcoRI. The restriction assay was purified with the NucleoSpin Gel and PCR Clean-up Kit (Macherey Nagel, Düren). For the assembly of the DNA fragments by means of Gibson Assembly (Gibson et al., 2009a), the amplified fragments were used in threefold molar excess over the linearized vector backbone pK19mobsacB. The DNA fragments were provided with a prepared Gibson Assembly Master Mix which, in addition to an isothermal reaction buffer, contains the enzymes required for assembly (T5 exonuclease, phusion DNA polymerase and Taq DNA ligase). The assembly of the fragments is carried out at 50° C. for 60 minutes in a thermal cycler. After assembly of the fragments, the total batch volume was used for transformation of chemically competent E. coli DH5a cells by heat shock at 42° C. for 90 seconds. Following the heat shock, the cells were regenerated on ice for 90 seconds before being provided with 800 μL LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/mL) and incubated overnight at 37° C. The correct assembly of the mutation plasmids in the grown transformants was verified by colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct assembly of the fragments used, forms a PCR product of a specific size which was checked by gel electrophoresis. Clones whose PCR product indicates a correct assembly of the deletion plasmid pK19mobsacB-AfasB were grown overnight in LB medium with kanamycin (50 pg/mL) for isolation of the plasmids. The plasmids were then isolated with the NucleoSpin Plasmid (NoLid) Kit (Macherey-Nagel, Düren) and sequenced with said amplification and colony PCR primers.

An aliquot of electrocompetent C. glutamicum cells was transformed with the described protocol with the respective deletion plasmid and spread on BHIS Kan¹⁵ plates. Since the pK19mobsacB plasmid cannot replicate in C. glutamicum, the subsequent selection of the mediated kanamycin resistance could be expected to be formed only if the deletion plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the deletion plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the deletion plasmid into their genome via homologous recombination are resistant to kanamycin and sensitive to sucrose.

The excision of pK19mobsacB took place in a second recombination event via the now doubly present DNA regions in which the codon to be mutated from the chromosome was eventually exchanged for the introduced mutation fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired deletion. The successful deletion in the clones obtained after excision was checked for the expected fragment size upon deletion of the clones obtained by means of colony PCR. The primers chk-fasB-s/chk-fasB-as used were selected here in such a way that they bind in the chromosome outside of the deleted DNA region and also outside of the amplified flanking gene regions.

Primers used
fasB-(cg2743)-up-s:
ATCCCCGGGTACCGAGCTCGAATTCGCGATTTCGATGCCT
GGATG
fasB-(cg2743)-up-as:
CGCGGGAATCGAAGTTCCTGCTCAATTCGG
fasB-(cg2743)-down-s:
CAGGAACTTCGATTCCCGCGCCCGCCTA
fasB-(cg2743)-down-as:
TTGTAAAACGACGGCCAGTGAATTCGATACTGCAATATCAA
ACCAAG ATCTCCATT CTCC
chk-fasB-s:
GGAGGAT ACAT CCACGGT CATT G
chk-fasB-as:
CGCTATGAGTT CAGGAT GTT GAT CG

Nucleotide Substitution in the Gene fasB Coding for a Fatty Acid Synthase with Reduced Functionality for the Integration into the Genome of Coryneform Bacteria Cells

C. glutamicum DelAro⁴-4cl_{Pc} cells were grown in 5 ml BHI medium (test tube, 30° C., 170 rpm) to an OD₆oonm of 5 to ensure that the exponential growth phase was reached. Whole cell mutagenesis was performed by the addition of methylnitronitrosoguanidine (MNNG) dissolved in DMSO (final concentration 0.1 mg/mL) for 15 minutes at 30° C. The treated cells were washed twice with 45 ml NaCl, 0.9% (w/v), resuspended in 10 ml BHI medium and then regenerated for 3 hours at 30° C. and 170 rpm. The mutant cells were stored as glycerol stocks at −30° C. in BHI medium containing 40% (w/v) glycerol. For the determination of the malonyl-CoA provision, dilutions of the cell libraries were plated onto BHI agar plates so that individual colonies could be picked. Individual clones were randomly picked and cultured for the determination of malonyl-CoA provision according to the LC MS/MS protocol described. Subsequently, the genome of the clones for which an improved provision of malonyl-CoA could be measured was sequenced. To determine which of the detected mutations contribute to improved malonyl-CoA provision, selected mutations were integrated into the stem root C. glutamicum DelAro⁴-4cl_{Pc}. A re-measurement of the malonyl-CoA provision by LC MS/MS was performed to check whether the introduced mutations provided the putative positive influence on malonyl-CoA provision.

Construction of the Plasmids pK19mobsacB-fasB-E622K, pK19mobsacB-fasB-G1361D, pK19mobsacB-fasB-G2153D and pK19mobsacB-fasB-G2668S

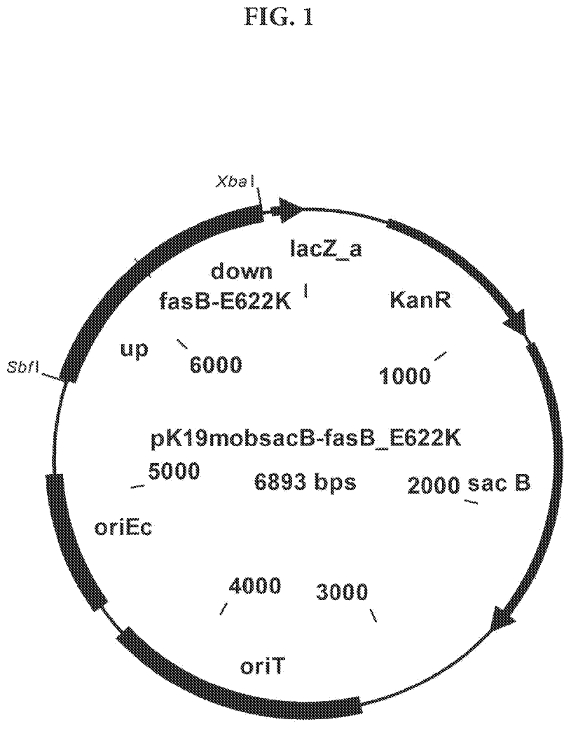

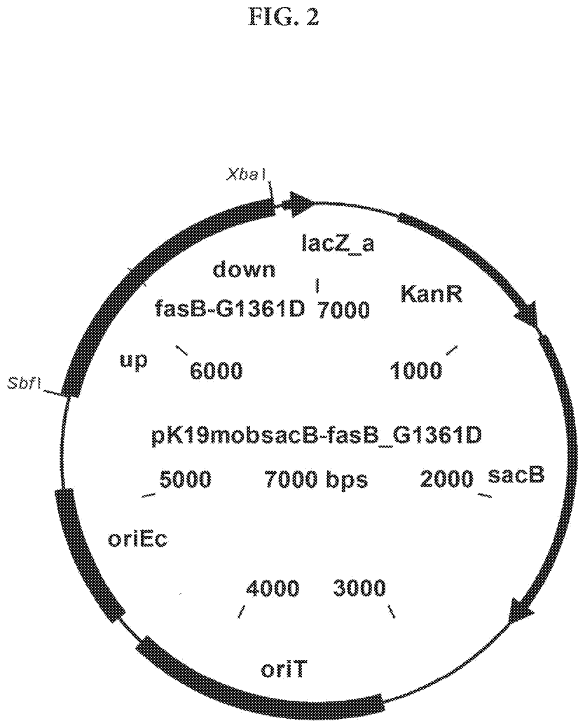

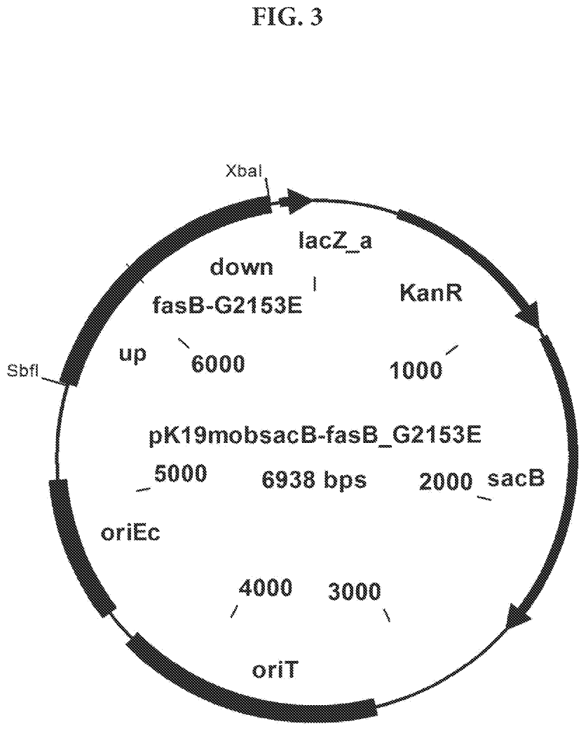

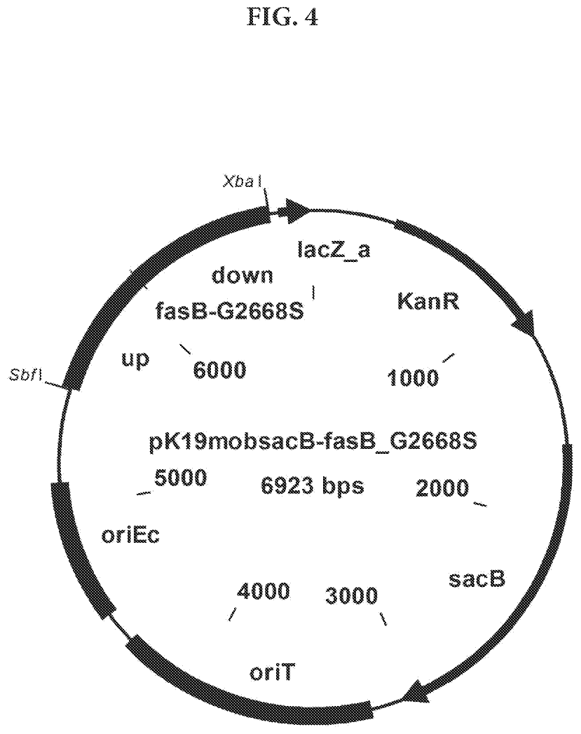

For the construction of the plasmids pK19mobsacB-fasB-E622K (FIG. 1), pK19mobsacB-fasB-G1361 D (FIG. 2), pK19mobsacB-fasB-G2153D (FIG. 3) and pK19mobsacB-fasB-G2668S (FIG. 4) for the integration of the respective amino acid substitution in the fatty acid synthase B that is coded in C. glutamicum by means of the gene fasB, the flanking fragments of the respective codon to be mutated for the homologous recombination event were amplified by PCR starting from isolated genomic C. glutamicum DNA.

For the generation of the upstream fragment, the primer pair Sbfl_XXX_s/OL_XXX_as was used, the downstream flank was amplified with the primer pair OL_XXX_s/Xbal_XXX-as. The coding XXX in each case stands for the amino acid substitution to be inserted at a specific position in the fatty acid synthase B. The verification of the generated DNA fragments for the expected base pair size was carried out by means of gel electrophoretic analysis on a 1% agarose gel. The nucleotide sequences of the inner primers (OL_XXX_as/OL_XXX_s) facing the codon to be mutated were selected such that the two amplified fragments up and down contain complementary overhangs. In a second PCR (without addition of DNA primers), the purified fragments attach via the complementary sequences and serve as both primers and templates for each other (overlap extension PCR). The mutation fragment thus generated was amplified in a final PCR with the two exterior (facing away from the gene) primers from the first PCR (Sbfl_XXX_s/Xbal_XXX-as). After electrophoretic separation on a 1% TAE agarose gel, the final mutation fragment was isolated from the gel with the NucleoSpin® Gel and PCR Clean-up Kit (Macherey-Nagel, Düren) according to the accompanying protocol. For the construction of the mutation plasmids, both the mutation fragments and the empty vector pK19-mobsacB were linearized with the FastDigest variants (Thermo Fisher Scientific) of the restriction enzymes Sbf\ and Xba\. The restriction assays of said fragments were purified with the NucleoSpin Gel and PCR Clean-up Kit (Macherey Nagel, Düren). For the ligation of the hydrolyzed DNA fragments by means of the Rapid DNA Ligation Kit (Thermo Fisher Scientific), one mutation fragment respectively was used in threefold molar excess relative to the linearized vector backbone pK19mobsacB. After ligation of the fragments, the total batch volume was used for transformation of chemically competent E. coli DF15a cells by means of heat shock at 42° C. for 90 seconds. Following the heat shock, the cells were regenerated on ice for 90 seconds before being provided with 800 μL LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/ml) and incubated overnight at 37° C. The correct assembly of the mutation plasmids in the grown transformants was verified by colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct ligation of the fragments used, forms a PCR product of a specific size which was checked by gel electrophoresis. Clones whose PCR product indicates a correct assembly of the respective mutation plasmid pK19mobsacB-fasB-XXX were grown overnight in LB medium with kanamycin (50 pg/mL) for isolation of the plasmids. The plasmids were then isolated with the NucleoSpin Plasmid (NoLid) Kit (Macherey-Nagel, Düren) and sequenced with said amplification and colony PCR primers.

An aliquot of electrocompetent C. glutamicum cells was transformed with the described protocol with the respective mutation plasmid and spread on BHIS Kan¹⁵ plates. Since the pK19mobsacB plasmid cannot replicate in C. glutamicum, the subsequent selection of the mediated kanamycin resistance could be expected to be formed only if the mutation plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the mutation plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the mutation plasmid into their genome via homologous recombination are resistant to kanamycin and sensitive to sucrose.

The excision of pK19mobsacB took place in a second recombination event via the now doubly present DNA regions in which the codon to be mutated from the chromosome was eventually exchanged for the introduced mutation fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired mutation. For the detection of the successful mutation in the clones obtained after excision, the corresponding genomic region was amplified by colony PCR (primer pair Sbfl_XXX_s/Xbal_XXX-as). The PCR products were cleaned with the NucleoSpin Gel and PCR Clean-up-Kit (Macherey-Nagel, Düren) and sequenced to verify the mutation with the primers Sbfl_XXX_s, OL_XXX_as, OL_XXX_s and Xbal_XXX-as.

Primers used:
univ:
CGCCAGGGTTTTCCCAGTCACGAC
rsp:
CACAGGAAACAGCTATGACCATG
pK19mobsacB-fasB-E622K
OL_622-s:
GTACCGCT GCGAT GGCAACCAAGAAAGCAACCACCT CCCAG
GCCGTC
OL 622-as:
GACGGCCTGGGAGGTGGTTGCTTTCTTGGTTGCCATCGCAGCGG
TAC
Sbfl 622-s:
AAAACCTGCAGGGGCTGAGCTCGCTGGTGGCGGACAGGTTACCC
CAG
Xbal 622-as:
GGGGTCT AG AACGT CCTTATCAATGACGGGCACAAAGTT
CACAGGC
pK19mobsacB-fasB-G1361 D
OL 1361-s:
CCTCACCCAGTTCACCCAGGTGGACATGGCAACTCTGGGCGTTG
CTC
OL 1361-as:
GAGCAACGCCCAGAGTTGCCATGTCCACCTGGGTGAACTGGGTG
AGG
Sbfl 1361-s:
AAAACCTGCAGGGTTGCACCTGAATCCATGCGCCCATTCGCTGTG
ATC
Xbal 1361-as:
GGAATCTAGATCGGCGGAAGCAGCCTTGAAATCAGCCAAGATCTC
pK19mobsacB-fasB-G2153D
OL 2153-s:
CATTCGCGGCACCTCGCGTGTCCGAATCCATGGCAGATGCAGGC
CCAC
OL 2153-as:
GTGGGCCTGCATCTGCCATGGATTCGGACACGCGAGGTGCCGCG
AATG
Sbfl 2153-s:
AAAACCTGCAGGTTGGCCACGTCAGGTTGCACCAAGCTTCGATGA
AG
Xbal 2153-as:
AAAATCTAGACCGAGCTCGCCGGCGCCAACGATGACGACCATCT
CG
pK19mobsacB-fasB-G2668S
OL_G2668S-s:
AGTCCGACTTCGTTGTCGCATCCGGCTTCGATGCCCTGTCC
OL_G2668S-as:
GGACAGGGCATCGAAGCCGGATGCGACAACGAAGTCGGACT
Sbfl G2668S-s:
AAAACCTGCAGGCACTGACCTACGTCGACTCCGAGCCAGAACTCA
C
Xbal G2668S-as:
GGGGTCTAGATGCGCAGCCAGACGAGGTGGGAATGCTTGGACAG

Also included in variants of the present invention are proteins of the fatty acid synthase FasB of coryneform bacteria and/or nucleic acid sequences encoding a fatty acid synthase FasB of coryneform bacteria in which there are nucleotide substitutions and respective corresponding amino acid substitutions. Such variants are described, for example, in SEQ ID NO. 1 with a nucleotide substitution at position 1864 (g->a), in SEQ ID NO. 3 with a nucleotide substitution at position 4082 (g->a), in SEQ ID NO. 5 with a nucleotide substitution at position 6458 (g->a), in SEQ ID NO. 7 with a nucleotide substitution at the positions 8002-8004 (ggt->tcc) and in SEQ ID NO. 9 with a deletion of positions 25-8943.

General Methodology for the Deletion or Mutation (Nucleotide Substitution) of Genes or Integration of DNA into Coryneform Bacteria Cells

The following steps are identical for both deletions and integration/substitutions. For the sake of simplicity, only deletion strains or deletion plasmids are mentioned.

For the construction of C. glutamicum deletion strains, pK19/77obsacB-based deletion plasmids are cloned (Schafer et al. 1994; https://doi.org/10/1016/0378: 1119(94)90324-7). The target gene is then deleted as described (Niebisch & Bott, 2001; https://doi.org/10.1007/s002030100262). The deletion fragment required for this is generated by means of cross-over PCR (Link et al., 1997; https://doi.org/10.1 128/jb.179.20.6228-6237.1997). For this purpose, in the first step, flanking fragments are generated in two separate reactions of −500 bp, which fragments lie in the chromosome upstream and downstream of the gene to be deleted. The nucleotide sequences of the inner primers (facing the gene to be deleted) are selected in such a way that the two amplified fragments contain mutually complementary overhangs. In a second PCR, the purified fragments attach via the complementary sequences and mutually serve both as primers and as template. The deletion fragment thus generated is amplified in a final PCR with the two exterior (facing away from the gene) primers from the first PCR. After electrophoretic separation on a 1% TAE agarose gel (Sambrook et al., 1989), the final deletion fragment is isolated from the gel with the NucleoSpin® Gel and PCR Clean-up Kit (Macherey-Nagel, Düren) according to the accompanying protocol. The deletion fragment is subsequently ligated to the vector pK19mobsacB via the inserted and hydrolyzed restriction sites. Subsequently, chemically competent E. coli DH5 cells are transformed with the entire ligation assay. The grown transformants are checked for the correct ligation product by means of colony PCR; positive deletion plasmids are isolated and sequenced. In the case of insertions, the DNA sequence to be inserted is cloned between the flanking regions of the target locus. The following steps are identical for both deletions and insertions. For the sake of simplicity, only deletion plasmids are mentioned.

An aliquot of electrocompetent C. glutamicum cells is transformed with the described protocol with the respective deletion plasmid and spread on BHIS Kan¹⁵ plates. Since the pK19mobsacB plasmid cannot replicate in C. glutamicum, the subsequent selection of the mediated kanamycin resistance could be expected to be formed only if the deletion plasmid could be successfully integrated into the genome of C. glutamicum via the homologous sequences. The resulting integrants are plated in a first round of selection onto BHI Kan²⁵ plates as well as BHI 10% sucrose (w/v) plates and incubated overnight at 30° C. Successful genome integration of the deletion plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996; PMID 8899981). Thus, colonies that have integrated the deletion plasmid into their genome via homologous recombination are resistant to kanamycin and sensitive to sucrose.

The excision of pK19mobsacB takes place in a second recombination event via the now doubly present DNA regions in which the codon to be deleted from the chromosome is eventually exchanged for the introduced mutation fragment. For this purpose, cells showing the described phenotype (kanamycin-resistant, sucrose-sensitive) are incubated in a test tube with 3 ml BHI medium (without the addition of kanamycin) for 3 hours at 30° C. and 170 rpm. Subsequently, 100 μl of a 1:10 dilution are each spread onto BHI Kan²⁵ plates and BHI 10% sucrose (w/v) plates and incubated overnight at 30° C. In total, 50 of the clones grown on the BHI 10% sucrose (w/v) plate are selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired gene deletion. The successful deletion in the clones obtained after excision is checked for the expected fragment size upon deletion of the gene or gene cluster obtained by means of colony PCR. The primers used are selected here in such a way that they bind in the chromosome outside the deleted DNA region and also outside of the amplified flanking gene regions.

With this procedure described above, strains are constructed starting from the strain C. glutamicum ATCC 13032, which, in the coding region of the homologous fatty acid synthase gene fasB, have nucleotide substitutions (C.g.130232-fasB-E622K, C.g.130232-fasB-G1361 D, C.g.130232-fasB-G2153E, C.g.130232-fasB-G2668S) and/or deleted regions (C.g 13032-AfasB), changes in the homologous FasO binding site before the gene cluster accBCDI (C.g.130232-mufasO), as well as a homologous promoter region with reduced activity upstream of the gene coding for the citrate synthase gtlA (C.g. 13032-C7). These strains are distinguished by the fact that they are modified non-recombinantly and are thus distinguished as non-GVO.

Strain Construction C. glutamicum 13032 DelAro³/DelAro⁴

The methodology described below was used for the construction of C. glutamicum DelA-ro⁴-4clp_{c}c_{g} as well as all corresponding intermediates such as, for example, C. glutamicum DelAro3, DelAro4 and DelAro³-4c/_{PoCg}.

The strain C. glutamicum MB001 (DE3) is selected as the starting strain for the construction of C. glutamicum DelAro⁴-4cl_{PcCg}. This is a prophage-free C. glutamicum ATCC13032 wild-type strain (Stamm C. glutamicum MB001; Baumgart et al, 2013b, https://doi.org/10.1 128/AEM.01634-13), which further possesses a chromosomally integrated T7 polymerase that allows the use of the strong and inducible T7 promoter (Stamm C. glutamicum MB001 (DE3); (Kortmann et al, 2015; https://doi.org/10.1111/1751-7915.12236). This promoter is also located on the pMK Ex2 plasmids used for the expression of genes of plant origin involved in the synthesis of the respective product.

Starting from C. glutamicum MB001 (DE3), the strain C. glutamicum DelAro³ is constructed by deletion of the gene (clusters) cg0344-47, cg2625-40 and cg1226 (Kallscheuer et al., 2016, https://doi.Org/10.1016/j.ymben.2016.06.003).

Here, cg0344-47 is the phdBCDE operon coding for genes involved in the catabolism of phenylpropanoids such as p-cumaric acid.

To prevent non-specific conversion of phenylpropanoids by enzyme catalyzed ring hydroxylation or ring cleaving reactions (the natural substrates of the respective enzymes 4-hydroxybenzoate-3-hydroxylase PobA and protocatechuate dioxygenase PcaGH respectively show high structural similarity to phenylpropanoids) the corresponding gene (clusters) cg 1226 (pobA) and cg2625-40 (cat, ben and pca genes essential for the degradation of 4-hydroxybenzoate, catechol, benzoate and protocatechuate) are deleted.

During the establishment of the synthesis of plant polyphenols from glucose (in addition to plasmid pEKEx3_aro/-/_{εc}_ta/_{Fj}), an accumulation of 0.9 g/L protocatechuate is measured, but neither L-tyrosine nor p-cumaric acid can be detected (Kallscheuer et al., 2016 https://doi.Org/10.1016/j.ymben.2016.06.003). The 3-dehydroshikimate dehydratase QsuB catalyzes the thermodynamically irreversible conversion of the shikimate path intermediates 3-dehydroshikimate to protocatechuate and thus leads to an undesired loss of intermediates of the synthetic pathway of aromatic amino acids. The deletion of qsuB reduced the accumulation of protocatechuate. Thus, in the constructed strain C. glutamicum DelAro³, the gene cg0502 (qsuB) is additionally deleted, resulting in strain C. glutamicum DelAro⁴.

The chromosomal integration of the 4cl_{PcC8} gene from Petroselinum crispum under control of the T7 promoter to the deletion locus cg0344-47 (Acg0344-47::P_{T7}-4c/_{PcCg}) in the strain C. glutamicum DelAro³ and/or DelAro⁴ served to construct C. glutamicum DelAro³-4c/_{PcCg} and/or C. glutamicum DelAro⁴-4c/_{PcCg}.

Starting from C. glutamicum DelAro³-4 cl_{pcC}g and/or. C. glutamicum DelAro⁴-4 cl_{Pc}c_{g}, the corresponding C. glutamicum strains are constructed in a similar way (see above deletion and/or integration of DNA into coryneform bacteria) by integration of non-recombinantly altered DNA, in which strains the gene of the fatty acid synthase fasB is mutated or deleted, the FasO binding site is mutated before the gene cluster accBCDI and/or the promoter is mutated before the citrate synthase gene gltA. By way of example for all the above-mentioned C. glutamicum strains (DelAro³, DelAro⁴, DelAro³-4c/_{PcCg}, DelAro⁴-4c/_{PcCg}), the strains with C. glutamicum DelAro⁴-4c/_{PcC8}-fasB-E622K, DelAro⁴-4c/_{PcCg}-fasB-G1361 D, DelAro⁴-4c/_{PcC8}-fasB-G2153E, DelAro⁴-4c/_{PcC8}-fasB-G2668S, DelAro⁴-4c/_{PcCg}-AfasB, DelAro⁴-4c/_{PcC8}-C7, DelAro⁴-4c/_{PcCg}-C7-mufasO, DelAro⁴-4c/_{PcCg}-C7-mufasO-fasB-E622K, DelAro⁴-4c/_{PcCg}-C7-mufasO-fasB-G1361 D, DelAro⁴-4c/_{PcC8}-C7-mufasO-fasB-G2153E, DelAro⁴-4c/_{PcC8}-C7-mufasO-fasB-G2668S, DelAro⁴-4c/_{PcC8}-C7-mufasO-AfasB are constructed in this way. All other conceivable bacterial strains with combinations of changes in genes of coryneform bacteria such as, for example, fasB, fasO and gtlA in the genome of the C. glutamicum wild-type ATCC 13032 or its derivatives C. glutamicum DelAro³, C. glutamicum DelAro⁴, C. glutamicum DelAro³-4c/_{PcCg}, C. glutamicum DelAro⁴-4 cl_{PcCg} are also produced in the same manner as described.

Construction of pK19mobsacB-cg0344-47-del and pK19mobsacB-cg2625-40-del

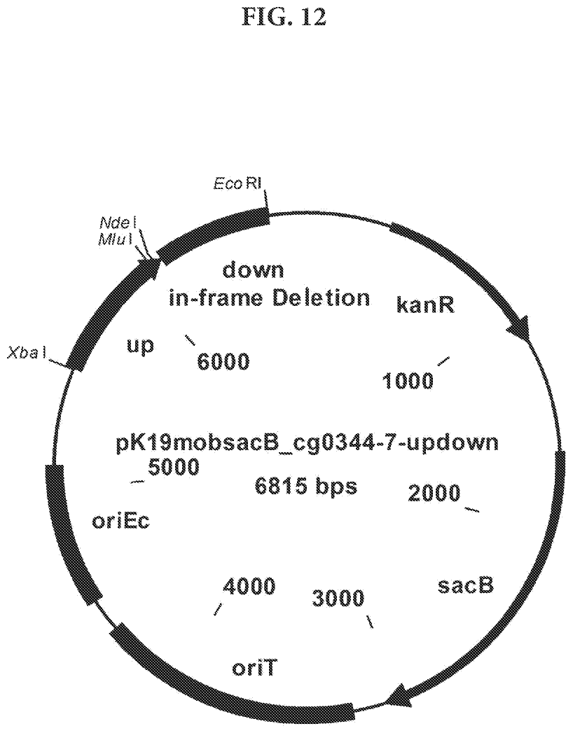

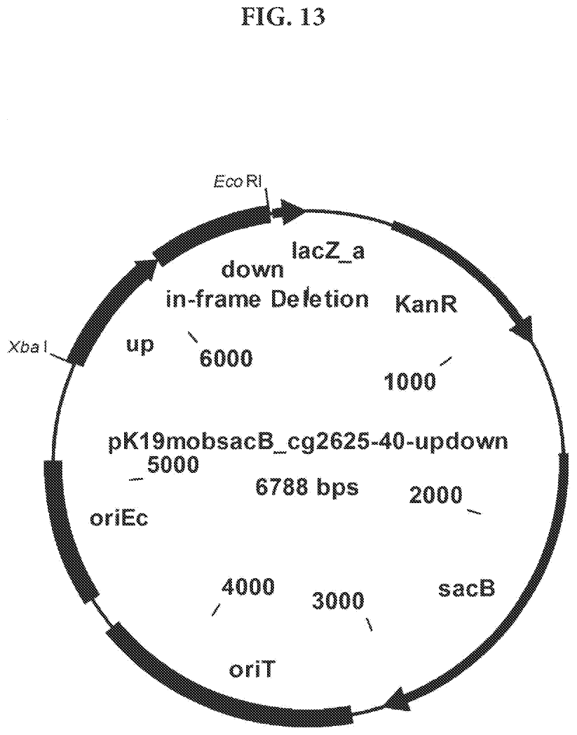

To construct the plasmid pK19mobsacB-cg0344-47-del (FIG. 12) and pK19mobsacB-cg2625-40-del (FIG. 13) for the deletion of the gene clusters cg0344-47 and cg2625-40 in C. glutamicum, the flanking fragments of each of the gene clusters to be deleted required for the homologous recombination event were amplified by PCR starting from isolated genomic C. glutamicum DNA.

For the generation of the upstream fragment, the primer pair cgXXXX-XX-up-s/cgXXXX-XX-up-as was used, the downstream flank was amplified with the primer pair cgXXXX-XX-down-s/cgXXXX-XX-down-as. The coding XXXX-XX here represents in each case the cg numbers of the genes to be deleted. For example, for the deletion of the gene cluster cg0344-47, the primer pair cg0344-47-up-s/cg0344-47-up-as is used and similarly for the deletion of the gene cluster cg2625-40, the primer pair cg2625-40-up-s/cg2625-40-up-as is used. The check of the generated DNA fragments for the expected base pair size was performed by means of gel electrophoretic analysis on a 1% agarose gel. The nucleotide sequences of the inner primers (facing the gene to be deleted) (cgXXXX-XX-up-as/cgXXXX-XX-down-s) were selected in such a way that the two amplified fragments up and down contain mutually complementary overhangs. For the gene cluster cg0344-47 this is the primer pair cg0344-47-up-as/cg0344-47-down-s and similarly for the gene cluster cg2625-40 the primer pair cg2625-40-up-as/cg2625-40-down-s. In a second PCR (without addition of DNA primers), the cleaned fragments attach via the complementary sequences and mutually serve both as primers and as templates (overlap extension PCR). The deletion fragment thus generated was amplified in a final PCR with the two exterior (facing away from the gene) primers from the first PCR (cgXXXX-XX-up-s/cgXXXX-XX-down-as). For the gene cluster cg0344-47 this is the primer pair cg0344-47-up-s/cg0344-47-down-as and similarly for the gene cluster cg2625-40 the primer pair cg2625-40-up-s/cg2625-40-down-as. After electrophoretic separation on a 1% TAE agarose gel, the final deletion fragment was isolated from the gel with the NucleoSpin® Gel and PCR Clean-up Kit (Macherey-Nagel, Düren) according to the accompanying protocol. For the construction of the deletion plasmids, both the deletion fragments and the empty vector pK19-mobsacB were linearized with the FastDigest variants (Thermo Fisher Scientific) of the restriction enzymes Xba\ und EcoRI. The restriction assays of said fragments were purified with the NucleoSpin Gel and PCR Clean-up Kit (Macherey Nagel, Düren). For the ligation of the hydrolyzed DNA fragments by means of the Rapid DNA Ligation Kit (Thermo Fisher Scientific), one of the two deletion fragments was used in threefold molar excess relative to the linearized vector backbone pK19mobsacB. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL of LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/mL) and incubated overnight at 37° C. The correct assembly of the deletion plasmids in the grown transformants was verified by colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct ligation of the fragments used, forms a PCR product of a specific size which was checked by gel electrophoresis. Clones whose PCR product indicates a correct assembly of the mutation plasmids pK19mobsacB-cg0344-47-del and/or pK19mobsacB-cg2625-40-del were grown overnight in LB medium with kanamycin (50 pg/mL) for isolation of the plasmids. The plasmids were then isolated with the NucleoSpin Plasmid (NoLid) kit (Macherey-Nagel, Düren) and sequenced with said amplification and colony PCR primers.

An aliquot of electrocompetent C. glutamicum cells was transformed with the described protocol with the respective deletion plasmid and spread on BHIS Kan¹⁵ plates. Since the pK19mobsacB plasmid cannot replicate in C. glutamicum, the subsequent selection of the mediated kanamycin resistance could be expected to be formed only if the deletion plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the deletion plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the deletion plasmid into their genome via homologous recombination are resistant to kanamycin and sensitive to sucrose.

The excision of pK19/77obsacB took place in a second recombination event via the now doubly present DNA regions in which the gene to be deleted from the chromosome was eventually exchanged for the introduced deletion fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and % sucrose (w/v) was spread and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired gene deletion. The successful deletion in the clones obtained after excision was checked for the expected fragment sequence upon deletion of the gene or gene cluster by means of colony PCR of the clones obtained. The primers del-cgXXXX-XX-s/del-cgXXXX-XX-as used were selected here in such a way that they bind in the chromosome outside of the deleted DNA region and also outside of the amplified flanking gene regions. For the gene cluster cg0344-47, this is the primer pair del-cg0344-47-s/del-cg0344-47-as and similarly for the gene cluster cg2625-40 the primer pair del-cg2625-40-s/del-cg2625-40-as.

Primers used
univ:
CGCCAGGGTTTTCCCAGTCACGAC
rsp:
CAC AG G AAAC AG CTAT G ACC AT G
pK19mobsacB-cg0344-47-del
cg0344-47-up-s:
CTCTCTAGAGCGGTGGCGATGATGATCTTCGAG
cg0344-47-up-as:
AAGCATATGAGCCAAGTACTATCAACGCGTCAGGGCGACTTT
TCCATTGAGAGACATTTC
cg0344-47-down-s:
CTGACGCGTTGATAGTACTTGGCTCATATGCTTTTCCTCACCC
GCTTCTACGCTTAAAAG
cg0344-47-down-as:
GACGAATTCGTGTGGCCACCACCTCAATCTGTG
del-cg0344-47-s:
AGAGATTCACCCTCGGCGATGAG
del-cg0344-47-as:
GACCCGCAATGGTGTCGCCAG
pK19mobsacB-cg2625-40-del
cg2625-40-up-s:
ACATCTAGAGGTCGGCGAATCAAGCTCCATG
cg2625-40-up-as:
CGTCTCGAGTTCACATATGCAACGCGTGCTCAAGATGACAAT
ATCTTGAGGGTTCATTTTTTGATCCTTAATTTAG
cg2625-40-down-s:
TTGAGCACGCGTTGCATATGTGAACTCGAGACGGTCGGTGGA
GGCGACCAGGGATAAC
cg2625-40-down-as:
TCTGAATTCATCAAGGCCAATCATGATGAGTGCGAAAC
del-cg2625-40-s:
AAGAGGAGTTGATGGGATGGTCGAACAATC
del-cg2625-40-as:
GTTGGCATGCCAGCTTTGTGGGATG

Construction of pK19mobsacB-Acg0344-47::P_{T7}-4c/_{Pc}

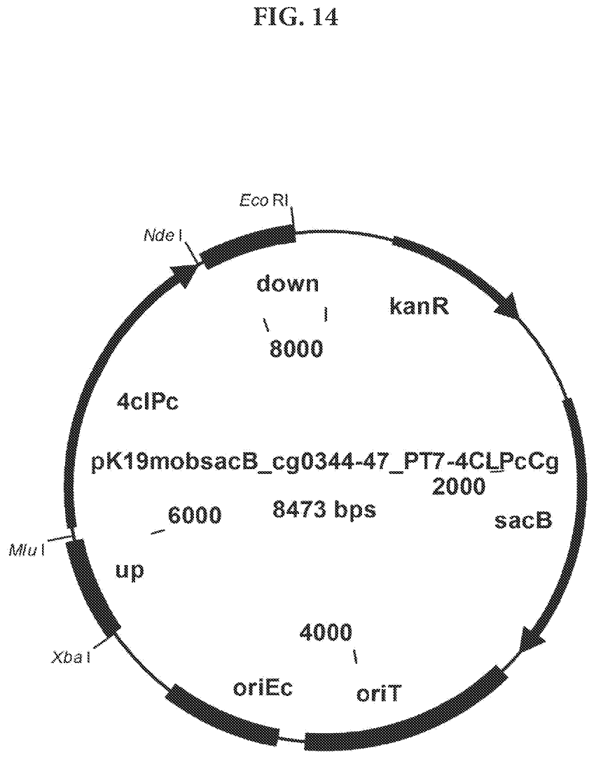

For the construction of the plasmid pK19mobsacB-Acg0344-47::P_{T7}-4c/_{Pc} (FIG. 14) for the chromosomal integration at the deletion locus cg0344-47 of a codon-optimized variant for C. glutamicum of the 4cl gene from Petroselinum crispum under the control of the T7 promoter (PT7-4C/_{Pc}), the gene from GeneArt Gene Synthesis (Thermo Fisher Scientific) was chemically synthesized as a string DNA fragment and used as DNA template for the amplification with the primer pair Mlul-PT7-4CLPcCg-s/Ndel-4CLPcCg-as. For the construction of the integration plasmid, both the amplified 4c/ₚ, gene and the plasmid pK19mobsacB-cg0344-47-del were linearized with the FastDigest variants (Thermo Fisher Scientific) of the restriction enzymes Mlu\ and Nde I. The restriction assays of said fragments were purified with the NucleoSpin Gel and PCR Clean-up Kit (Macherey Nagel, Düren). For ligation of the hydrolyzed DNA fragments by means of the Rapid DNA Ligation-Kit (Thermo Fisher Scientific), the 4cl_{Pc} fragment was used in threefold molar excess over the linearized vector backbone pK19mobsacB-cg0344-47-del. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/mL) and incubated overnight at 37° C. The correct assembly of the insertion plasmid in the grown transformants was verified by colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct ligation of the fragments used, forms a PCR product of a specific size which was checked by gel electrophoresis. Clones whose PCR product indicates a correct assembly of the insertion plasmid pK19mobsacB-Acg0344-47::P_{T7}-4c/_{Pc} were grown overnight in LB medium with kanamycin (50 pg/mL) for isolation of the plasmids. The plasmids were then isolated with the NucleoSpin Plasmid (NoLid) kit (Macherey-Nagel, Düren) and sequenced with said amplification and colony PCR primers.

An aliquot of electro-competent C. glutamicum cells was transformed with the described protocol with the insertion plasmid and spread on BHIS Kan¹⁵ plates. Since the pK19mobsacB plasmid cannot replicate in C. glutamicum, the subsequent selection of the mediated kanamycin resistance could be expected to be formed only if the mutation plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the insertion plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the insertion plasmid into their genome via homologous recombination are resistant to kanamycin and sensitive to sucrose.

The excision of pK19mobsacB took place in a second recombination event via the now doubly present DNA regions in which the integration locus selected from the chromosome was eventually exchanged for the introduced insertion fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired P_{T7}-4c/pc insertion. Successful insertion in the resulting clones after excision was checked for the expected fragment sequence upon insertion of the gene or gene cluster by means of colony PCR of the clones obtained. The primers del-cg0344-47-s/del-cg0344-47-as used were selected here in such a way that they bind in the chromosome outside of the insertion locus and also outside of the amplified flanking gene regions. PCR fragments indicating insertion of the PT7-4C/_{Pc} construct were cleaned with the NucleoSpin Gel and PCR Clean-up-Kit (Macherey-Nagel, Düren) and sequenced with the primers del-cg0344-47-s, cg0344-47-up-s, Mlul-PT7-4CLPcCg-s, Ndel-4CLPcCg-as, cg0344-47-down-as and del-cg0344-47-as to control insertion.

Primers used
univ:
CGCCAGGGTTTTCCCAGTCACGAC
rsp:
CACAGGAAACAGCTATGACCATG
Mlul-PT7-4CLPcCg-s:
TCCTACGCGTTAATACGACTCACTATAGGGAGATCAAGG
AGGCGGACAATGGGCGATTGCGTGGCAC
Ndel-4CLPcCg-as:
GGACGTTCATATGTTACTTTGGCAGATCACCGGATGCGA
TC
del-cg0344-47-s:
AGAGATTCACCCTCGGCGATGAG
cg0344-47-up-s:
CTCTCTAGAGCGGTGGCGATGATGATCTTCGAG
cg 0344-47-down-as:
GACGAATTCGTGTGGCCACCACCTCAATCTGTG
del-cg0344-47-as:
GACCCGCAATGGTGTCGCCAG

Construction of pK19mobsacB-cg0502-del

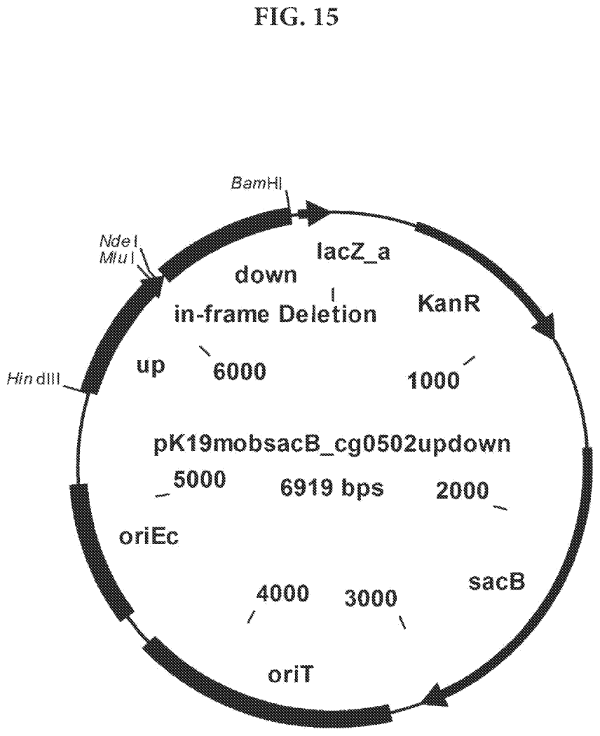

To construct the plasmid pK19mobsacB-cg0502-del (FIG. 15) for the deletion of the gene cg0502 in C. glutamicum, the flanking fragments required for the homologous recombination event were amplified by PCR starting from isolated genomic C. glutamicum DNA.

For the generation of the upstream fragment, the primer pair cg0502-up-s/cg0502-up-as was used, the downstream flank was amplified with the primer pair cg0502-down-s/cg0502-down-as. The check of the generated DNA fragments for the expected base pair size was performed by means of gel electrophoretic analysis on a 1% agarose gel. The nucleotide sequences of the inner primers (facing the gene to be deleted) (cg0502-up-as/cg0502-down-s) were selected in such a way that the two amplified fragments up and down contain mutually complementary overhangs. In a second PCR (without addition of DNA primers), the purified fragments attach via the complementary sequences and serve as both primers and templates for each other (overlap extension PCR). The deletion fragment thus generated was amplified in a final PCR with the two exterior (facing away from the gene) primers from the first PCR (cg0502-up-s/cg0502-down-as). After electrophoretic separation on a 1% TAE agarose gel, the final deletion fragment was isolated from the gel with the NucleoSpin® Gel and PCR Clean-up Kit (Macherey-Nagel, Düren) according to the accompanying protocol. For the construction of the deletion plasmids, both the deletion fragments and the empty vector pK19-mobsacB were linearized with the FastDigest variants (Thermo Fisher Scientific) of the restriction enzymes Hind\\\ and BamHI. The restriction assays of said fragments were cleaned with the NucleoSpin Gel and PCR Clean-up kit (Macherey-Nagel, Düren). For the ligation of the hydrolyzed DNA fragments by means of the Rapid DNA Ligation Kit (Thermo Fisher Scientific), the deletion fragment was used in threefold molar excess relative to the linearized vector backbone pK19mobsacB. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL of LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/ml) and incubated overnight at 37° C. The correct assembly of the deletion plasmids in the grown transformants was verified by colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct ligation of the fragments used, forms a PCR product of a specific size which was checked by gel electrophoresis. Clones whose PCR product indicates a correct assembly of the deletion plasmid pK19mobsacB-cg0502-del were grown overnight in LB medium with kanamycin (50 pg/mL) for isolation of the plasmids. The plasmids were subsequently isolated with the NucleoSpin Plasmid (NoLid) kit (Macherey Nagel, Düren) and sequenced with said amplification and colony PCR primers.

An aliquot of electrocompetent C. glutamicum cells was transformed with the described protocol with the respective deletion plasmid and spread on BHIS Kan¹⁵ plates. Since the pK19mobsacB plasmid cannot replicate in C. glutamicum, the subsequent selection of the mediated kanamycin resistance could be expected to be formed only if the mutation plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the deletion plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the deletion plasmid into their genome via homologous recombination are resistant to kanamycin and sensitive to sucrose.

The excision of pK19mobsacB took place in a second recombination event via the now doubly present DNA regions in which the gene to be deleted from the chromosome was eventually exchanged for the introduced deletion fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired gene deletion. The successful deletion in the clones obtained after excision was checked for the expected fragment sequence upon deletion of the gene or gene cluster by means of colony PCR of the clones obtained. The primers del-cg0502-s/del-cg0502-as used were selected here in such a way that they bind in the chromosome outside of the deleted DNA region and also outside of the amplified flanking gene regions.

Primers used
up-cg0502-s:
ACGAAGCTTTGTCCGGCATGCTGGCTGAC
up-cg0502-as:
TGCGCATATGTGGCCGTCTAGATACGCGTACGTCAAAC
AAACAGTGGCAATGGATGTACGCATG
down-cg0502-s:
ACGTACGCGTATCTAGACGGCCACATATGCGCAATCGA
GCGGGGAATCCCAAACTAGCATC
down-cg0502-as:
TATGGATCCTACGCCTGTACACCGTCGCACGTC
del-cg0502-s:
GTGAACATTGTGTTTACTGTGTGGGCACTGTC
del-cg0502-as:
TGATGTTCAGGCCGTTGAAGCCAAGGTAGAG
univ:
CGCCAGGGTTTTCCCAGTCACGAC
rsp:
CACAGGAAACAGCTATGACCATG

Construction of pK19mobsacB-cg1226-del

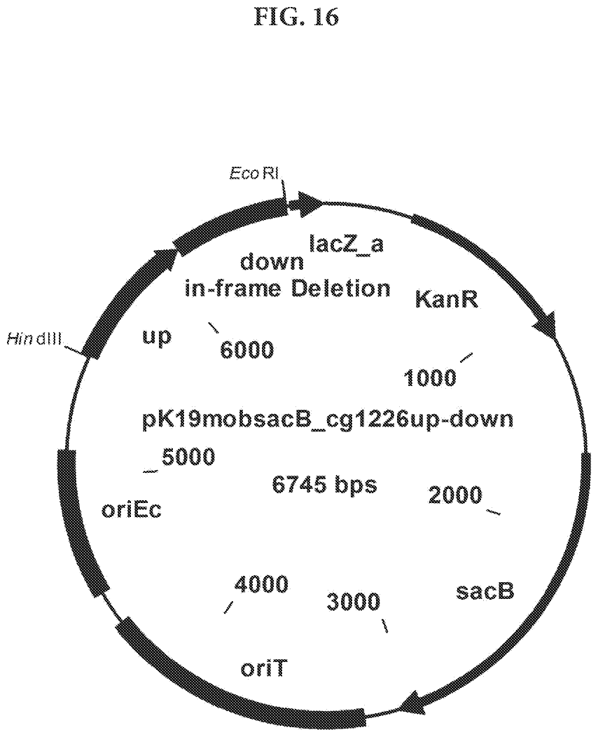

To construct the plasmid pK19mobsacB-cg1226-del (FIG. 16) for the deletion of the gene cg1226 in C. glutamicum, the flanking fragments required for the homologous recombination event were amplified by PCR starting from isolated genomic C. glutamicum DNA.

For the generation of the upstream fragment, the primer pair cg1226-up-s/cg1226-up-as was used, the downstream flank was amplified with the primer pair cg1226-down-s/cg1226-down-as. The check of the generated DNA fragments for the expected base pair size was performed by means of gel electrophoretic analysis on a 1% agarose gel. The nucleotide sequences of the inner primers (facing the gene to be deleted) (cg1226-up-as/cg1226-down-s) were selected in such a way that the two amplified fragments up and down contain mutually complementary overhangs. In a second PCR (without addition of DNA primers), the purified fragments attach via the complementary sequences and serve as both primers and templates for each other (overlap extension PCR). The deletion fragment thus generated was amplified in a final PCR with the two exterior (facing away from the gene) primers from the first PCR (cg1226-up-s/cg1226-down-as). After electrophoretic separation on a 1% TAE agarose gel, the final deletion fragment was isolated from the gel with the NucleoSpin® Gel and PCR Clean-up Kit (Macherey-Nagel, Düren) according to the accompanying protocol. For the construction of the deletion plasmids, both the deletion fragments and the empty vector pK19-mobsacB were linearized with the FastDigest variants (Thermo Fisher Scientific) of the restriction enzymes Hind and BamHI. The restriction assays of said fragments were cleaned with the NucleoSpin Gel and PCR Clean-up Kit (Macherey Nagel, Düren). For the ligation of the hydrolyzed DNA fragments by means of the Rapid DNA Ligation Kit (Thermo Fisher Scientific), the deletion fragment was used in threefold molar excess relative to the linearized vector backbone pK19mobsacB. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/ml) and incubated overnight at 37° C. The correct assembly of the deletion plasmids in the grown transformants was verified by colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair univ/rsp was used as DNA primer for the colony PCR, which specifically binds to the pK19mobsacB vector backbone and, in the case of correct ligation of the fragments used, forms a PCR product of a specific size which was checked by gel electrophoresis. Clones whose PCR product indicates a correct assembly of the deletion plasmid pK19mobsacB-cg1226-del were grown overnight in LB medium with kanamycin (50 pg/mL) for isolation of the plasmids. The plasmids were then isolated with the NucleoSpin Plasmid (NoLid) kit (Macherey-Nagel, Düren) and sequenced with said amplification and colony PCR primers.

An aliquot of electrocompetent C. glutamicum cells was transformed with the described protocol with the respective deletion plasmid and spread on BHIS Kan¹⁵ plates. Since the pK19mobsacB plasmid cannot replicate in C. glutamicum, the subsequent selection of the mediated kanamycin resistance could be expected to be formed only if the deletion plasmid could be successfully integrated into the genome of C. glutamicum via the homologous sequences. The resulting integrants were plated in a first round of selection onto BHI Kan²⁵ plates as well as BHI 10% sucrose (w/v) plates and incubated overnight at 30° C. Successful genome integration of the deletion plasmid results in the production of the levansucrase encoded by sacB in addition to kanamycin resistance. This enzyme catalyzes the polymerization of sucrose to the toxic levan, resulting in an induced lethality in growth on sucrose (Bramucci & Nagarajan, 1996). Thus, colonies that have integrated the deletion plasmid into their genome via homologous recombination are resistant to kanamycin and sensitive to sucrose.

The excision of pK19mobsacB took place in a second recombination event via the now doubly present DNA regions in which the gene to be deleted from the chromosome was eventually exchanged for the introduced deletion fragment. For this purpose, cells showing the described phenotype (kanamycin-resistant, sucrose-sensitive) were incubated in a test tube with 3 ml BHI medium (without the addition of kanamycin) for 3 hours at 30° C. and 170 rpm. Subsequently, 100 μl of a 1:10 dilution was each spread onto BHI Kan²⁵ plates and BHI 10% sucrose (w/v) plates and incubated overnight at 30° C. In total, 50 of the clones grown on the BHI 10% sucrose (w/v) plate were selected and spread on BHI Kan²⁵ as well as BHI 10% sucrose (w/v) to check the successful excision of pK19mobsacB and incubated overnight at 30° C. Should the plasmid have been completely removed, this results in sensitivity to kanamycin and resistance to sucrose of the particular clone. The second recombination event (excision) can also lead to the restoration of the wild-type situation in addition to the desired gene deletion. The successful deletion in the clones obtained after excision was checked for the expected fragment sequence upon deletion of the gene or gene cluster by means of colony PCR of the clones obtained. The primers del-cg1226-s/del-cg1226-as used were selected here in such a way that they bind in the chromosome outside of the deleted DNA region and also outside of the amplified flanking gene regions.

Primers used
up-cg1226-s:
CACAAGCTTCCACACGATGAAAAT
CAATCCGCAG
up-cg1226-as:
TGCGGTACCCTCGCATATGATATCTCGAGAG
CTAATTGCCACTGGTACGTGGTTCATG
down-cg1226-s:
AGCTCTCGAGATATCATATGCGAGGGTACCGC
AGACCTACCACGCTTCGAGGTATAAACGCTC
down-cg1226-as:
AGTGAATTCCAAGGAAGGCGGTTGCTACTGC
del-cg01226-s:
TAAATGGTGGAGATACCAAACTGTGAAGC
del-cg1226-as:
CGAGTTCTTCTTCGTGTTCGCGATC
univ:
CGCCAGGGTTTTCCCAGTCACGAC
rsp:
CACAGGAAACAGCTATGACCATG

### Codon Optimization of Heterologous Genes in Coryneform Bacteria Cells

The establishment of synthetic biosynthesis pathways, such as the synthesis of polyphenols or polyketides, from plants in coryneform bacteria cells requires a heterologous expression of the required plant genes. Different species are known to use variants of the universal genetic code with varying frequency, which is ultimately due to different tRNA concentrations within the cell. In this case, one speaks of codon usage. Codons rarely used can slow translation, while codons used more frequently can accelerate translation. This results in heterologous genes with codon usage being synthesized specifically for the target organism. To this end, the amino acid sequence of the heterologous protein of interest is rewritten into the DNA sequence with specific codon usage. For C. glutamicum, a database of codon usage is available at http://www.kazusa.or.ip/codon/cqi-bin/showcodon.cqi?species=196627&aa=1&style=N.

Expression of the aroH and Tal Genes in Coryneform Bacteria Cells
Construction of the Plasmid pEKEx3-aroH_{Ec}-fa/_{Fj}

In order to be able to carry out the synthesis of plant polyphenols without supplementation of the polyphenol precursor p-cumaric acid in coryneform bacteria, i.e. for the synthesis of plant polyphenols from glucose, two further genes are required (Kallscheuer et al., 2016; https://doi.Org/10.1016/j.ymben.2016.06.003). These are the genes coding for a feedback-resistant 3-deoxy-D-arabinoheptulosonate-7-phosphate synthase (aroH), preferably from E. coli (aroH_{εc}), and for a tyrosine ammonium lyase (tal), preferably from Flavobacterium johnsoniae (tal_{Fj}).

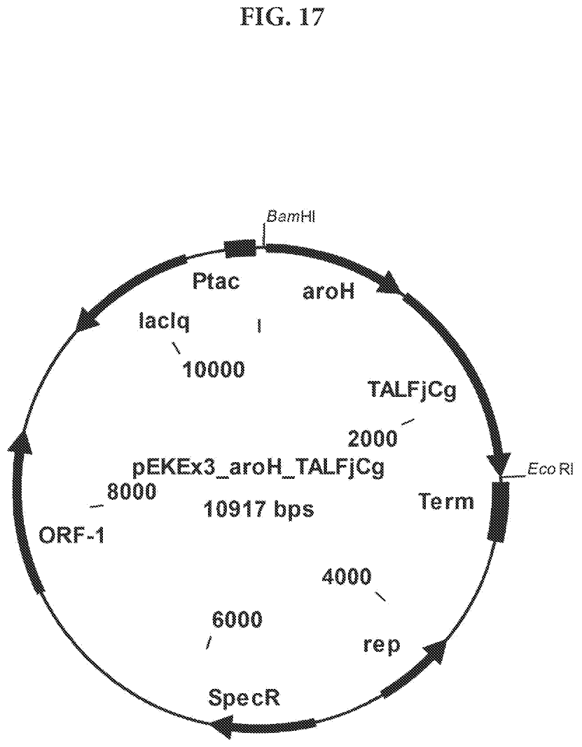

The procedure for the construction of the plasmid pEKEx3-aroH_{Ec}-tal_{Fj} (FIG. 17) is as follows:

For the construction of the plasmid pEKEx3-aroH_{Ec}-tal_{Fj}c_{g} for the expression of the genes aroH from E. coli (aroH_{Ec}) and a variant of the tal gene from Flavobacterium johnsoniae (tal_{FJCg}) codon-optimized for C. glutamicum, the two genes are amplified by PCR. For the aroH_{Ec} amplification by PCR, genomic DNA is isolated from E. coli and amplified with the primer pair aroHEc-s/aroHEc-as specific to that for the aroH_{Ec} gene. The tal_{FjC8} gene that is codon-optimized for C. glutamicum is chemically synthesized as a string DNA fragment by GeneArt Gene Synthesis (Thermo Fisher Scientific) and used as DNA template for the amplification of tal_{FJCg} with the primer pair talFj-s/talFj-as. The check of the generated DNA fragments for the expected base pair size is performed by means of gel electrophoretic analysis on a 1% agarose gel. For the construction of the plasmid pEKEx3-aroH_{Ec}-tal_{FjCg}, the plasmid pEKEx3 is linearized with the FastDigest variants (Thermo Fisher Scientific) of the restriction enzymes BamHI and EcoRI. The genes aroH_{Ec} and/or tal_{FjCg} amplified with the given primer pairs are hydrolyzed with the restriction enzymes of BamHI and SapI or SapI and EcoRI, respectively. The restriction assays of said fragments are purified with the NucleoSpin Gel and PCR Clean-up Kit (Macherey Nagel, Düren). For the ligation of the hydrolyzed DNA fragments by means of the Rapid DNA Ligation Kit (Thermo Fisher Scientific), the two inserts aroH_{E}, und tal_{FjCg} are used in threefold molar excess relative to the linearized vector backbone pEKEx3. After ligation of the fragments, the total batch volume is used for transformation of chemically competent E. coli DH5a cells by means of heat shock at 42° C. for 90 seconds. Following the heat shock, the cells are regenerated on ice for 90 seconds before being provided with 800 μL of LB medium and regenerated at 37° C. in a thermal mixer (Eppendorf, Hamburg) at 900 rpm for 60 minutes. Subsequently, 100 μL of the cell suspension was spread on LB agar plates with kanamycin (100 pg/mL) and incubated overnight at 37° C. The correct assembly of the inserted fragments in the grown transformants was checked by means of colony PCR. The 2× DreamTaq Green PCR Master Mix (ThermoFisher Scientific Inc., Waltham, Mass., USA) is used for this purpose. The DNA template was added to the PCR assay by adding cells of the grown colonies. By the initial denaturation step of the PCR protocol at 95° C. for 3 minutes, the cells are lysed so that the DNA template is released and is accessible for DNA polymerase. The primer pair chk_pEKEx3_s/chk_pEKEx3_as is used as DNA primer for the colony PCR, which specifically binds to the pEKEx3 vector backbone and, in the case of correct ligation of the fragments used, forms a PCR product of a specific size which is verified by gel electrophoresis. Clones whose PCR product indicates a correct assembly of the deletion plasmid pEKEx3-aroH_{Ec}-tal_{FjCg} are grown overnight in LB medium with kanamycin (100 pg/mL) for isolation of the plasmids. The plasmids are then isolated with the NucleoSpin Plasmid (NoLid) Kit (Macherey-Nagel, Düren) and sequenced with said amplification and colony PCR primers. This plasmid is shown in FIG. 1.

Primers used:
aroHEc-s:
CTCGGATCCAAGGAGGT CATAT
CATGAACAGAACGACGAACTCCGTACTGCGCGTATTG
aroHEc-as:
TACGCTCTTCTGATTTAGAAGCGGGTATCTACCGCAGAGGCGAG
talFj-s:
TTCGCTCTTCAATCTGGCAAGGAGGGATCCGTATGAACACCATCAA
CGAATACCTGTCCCTGGAAG
talFj-as:
ATCGAATT CTTAGTTGTTG ATCAGGTGATC CTTCACCTT CTG CAC
chk_pEKEx3_s:
GCAAAT ATT CTGAAAT GAGCTGTT GACAATT AAT CATC
chk_pEKEx3_as:
CGTTCT GATTT AAT CTGTAT CAGGCTGAAAAT CTTCTC

### Expression of Heterologous Genes for the Synthesis of Polyphenols or Polyketides in Coryneform Bacteria Cells

Construction of pMKEx2_sts_{Ah}_4cl_{Pc}

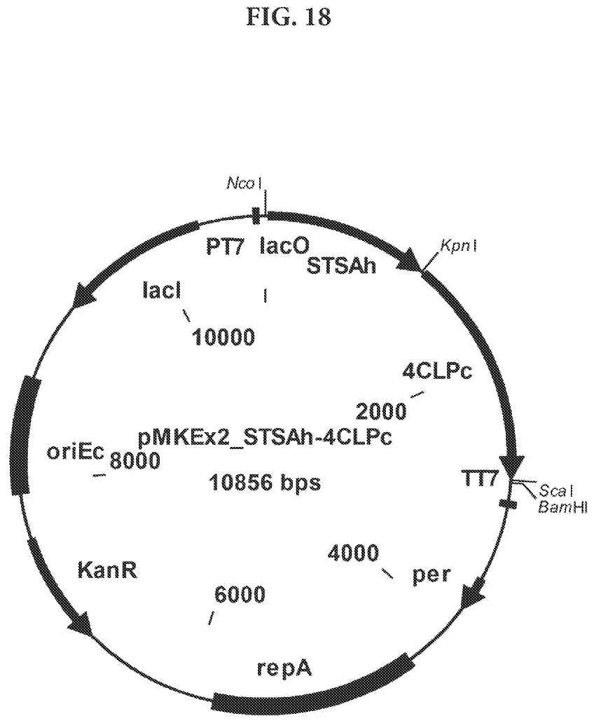

To construct the plasmid pMKEx2_sfs\hat{ }_4c/p, (FIG. 18) for the expression of the genes sfs from Arachis hypogea (sts_{Ah}) and 4cl from Petroselinum crispum (4cl_{pc}), the two genes were chemically synthesized by GeneArt Gene Synthesis (Thermo Fisher Scientific) as a string DNA fragment, as gen variants that are codon-optimized for C. glutamicum, and used as DNA template for amplification by PCR. The genes sts_{Ah} and 4cl_{Pc} were amplified by PCR with the primer pair stsAh-s/stsAh-as and/or 4clPc-s/4clPc-as, respectively, which is specific for the respective gene. The check of the generated DNA fragments for the expected base pair size was performed by means of gel electrophoretic analysis on a 1% agarose gel. For the construction of the plasmid pMKEx2_sts_{Ah}_4cl_{Pc}, the plasmid pMKEx2 is linearized with the FastDigest variants (Thermo Fisher Scientific) of the restriction enzymes NcoI and BamHI. The genes sts_{Ah} and 4cl_{Pc} amplified with the given primer pairs were hydrolyzed with the restriction enzymes NcoI and KpnI and/or KpnI and BamHI respectively. The restriction assays of said fragments were purified with the NucleoSpin Gel and PCR Clean-up Kit (Macherey Nagel, Düren). For the ligation of the hydrolyzed DNA fragments by means of the Rapid DNA Ligation Kit (Thermo Fisher Scientific), the two inserts sts_{Ah} and 4cl_{Pc}, were used in threefold molar excess relative to the linearized vector backbone pMKEx2. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL of LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/ml) and incubated overnight at 37° C. The correct assembly of the inserted fragments in the grown transformants was verified by means of colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair chk_pMKEx2_s/chk_pMKEx2_as was used as DNA primer for the colony PCR, which specifically binds to the pMKEx2 vector backbone and, in the case of correct ligation of the fragments used, forms a PCR product of a specific size which was checked by gel electrophoresis. Clones whose PCR product indicates a correct assembly of the plasmid pMKEx2_sts_{Ah}_4cl_{Pc} were grown overnight in LB medium with kanamycin (50 pg/mL) for isolation of the plasmids. The plasmids were then isolated with the NucleoSpin Plasmid (NoLid) Kit (Macherey-Nagel, Düren) and sequenced with said amplification and colony PCR primers.

Primers used
stsAh-s:
ATACCATGGTAAGGAGGACAGCTATGGTGTCCGTGTCCGGCATC
stsAh-as:
CTCGGTACCTTTAGATTGCCATAGAGCGCAGCACCAC
4clPc-s:
AGCGGTACCTAAGGAGGTGGACAATGGGCGATTGCGTGGCAC
4clPc-as:
CTGGGATCCAGGACTAGTTTCCAGAGTACTATTACTTTGGCAGAT
CACCGGATGCGATC
chk_pMKEx2-s:
CCCTCAAGACCCGTTTAGAGGC
chk_pMKEx2-as:
TTAATACGACTCACTATAGGGGAATTGTGAGC

Construction of pMKEX2-chs_{Ph}-chi_{Ph}

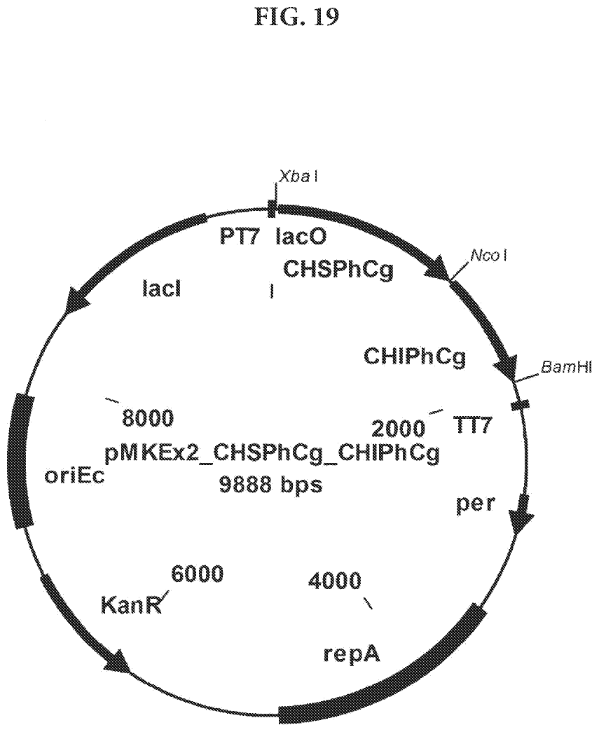

To construct the plasmid pMKEX2-chs_{Ph}-chi_{Ph} (FIG. 19) for the expression of the genes chs and chi from Petunia x hybrida (chs_{Ph} and chi_{Ph}), the two genes were chemically synthesized by GeneArt Gene Synthesis (Thermo Fisher Scientific) as a string DNA fragment, as gen variants that are codon-optimized for C. glutamicum, and used as DNA templates for amplification by PCR. The chs_{Ph} and chi_{Ph} were amplified by PCR with the primer pair chs_{Ph}-s/chsPh-as and/or chiPh-s/chiPh-as, which is specific for the respective gene. The check of the generated DNA fragments for the expected base pair size was performed by means of gel electrophoretic analysis on a 1% agarose gel. For the construction of the plasmid pMKEX2-chs_{Ph}-chi_{Ph}, the plasmid pMKEx2 is linearized with the FastDigest variants (Thermo Fisher Scientific) of the restriction enzymes Xba\ and Bam-HI. The genes chs_{Ph} and chi_{Ph} with the given primer pairs were hydrolyzed with the restriction enzymes Xba\ and Nco\ and/or Nco\ and BamHI, respectively. The restriction assays of said fragments were cleaned with the NucleoSpin Gel and PCR Clean-up Kit (Macherey Nagel, Düren). For the ligation of the hydrolyzed DNA fragments by means of the Rapid DNA Ligation Kit (Thermo Fisher Scientific), the two inserts chs_{Ph} and chi_{Ph} were used in threefold molar excess relative to the linearized vector backbone pMKEx2. After ligation of the fragments, the total batch volume was used for transformation of chemically competent E. coli DH5a cells by means of heat shock at 42° C. for 90 seconds. Following the heat shock, the cells were regenerated on ice for 90 seconds before being provided with 800 μL of LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/ml) and incubated overnight at 37° C. The correct assembly of the inserted fragments in the grown transformants was verified by means of colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair chk_pMKEx2_s/chk_pMKEx2_as was used as DNA primer for the colony PCR, which specifically binds to the pMKEx2 vector backbone and, in the case of correct ligation of the fragments used, forms a PCR product of a specific size which was checked by gel electrophoresis. Clones whose PCR product indicates a correct assembly of the plasmid pMKEx2_chs_{Ph} and chipₕ were grown overnight in LB medium with kanamycin (50 pg/mL) for isolation of the plasmids. The plasmids were then isolated with the NucleoSpin Plasmid (NoLid) Kit (Macherey-Nagel, Düren) and sequenced with said amplification and colony PCR primers.

Primers used
chsPh-s:
GTATCTAGAAAGGAGGTCGAAGATGGTGACCGTG
GAAGAATACCGCAAG
chsPh-as:
CTCCCATGGTTAGGTTGCCACGGAGTGCAGCAC
chiPh-s:
CTCCCATGGTGCTAAAGGAGGTCGAAGATGTCCC
CACCAGTGTCCGTGACCAAG
chiPh-as:
CTGGGATCCTTACACGCCGATCACTGGGATGGTG
chk_pMKEx2-s:
CCCTCAAGACCCGTTTAGAGGC
chk_pMKEx2-as:
TTAATACGACTCACTATAGGGGAATTGTGAGC

### Expression of the Polyketide Synthase PCS in Coryneform Bacteria Cells According to Embodiments of the Invention

Construction of pMKEx2-pcs_{Aa} and pMKEx2-pcs_{Aa}-short

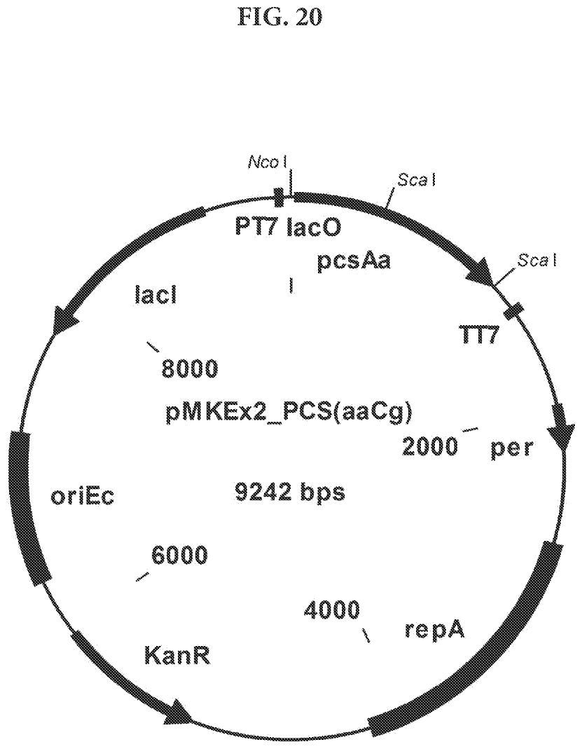

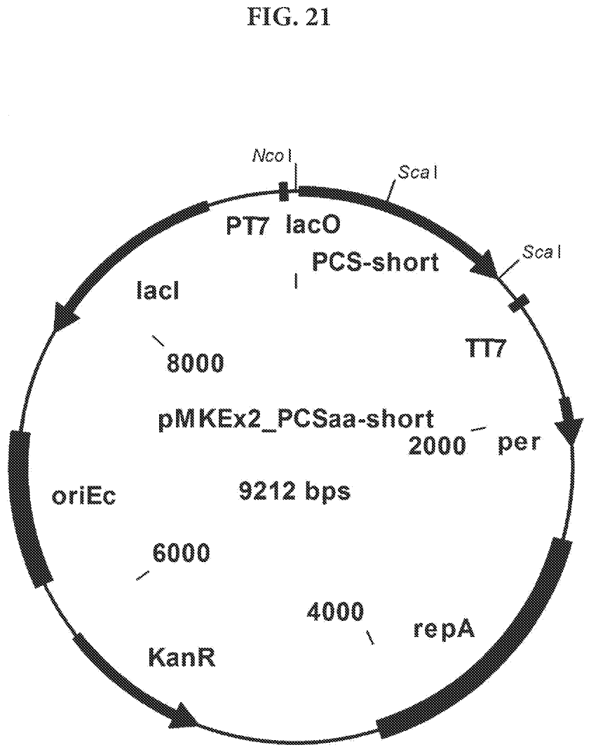

For the construction of the plasmids pMKEx2_pcs_{Aa} (FIG. 20) and pMKEx2\hat{ }cs\hat{ }-short (FIG. 21) for the expression of the gene variants from pcs from Aloe arborescens (pcs_{Aa}), the gene was chemically synthesized as a codon-optimized gene variant for C. glutamicum by GeneArt Gene Synthesis (Thermo Fisher Scientific) as a string DNA fragment and used as a DNA template for the amplification by PCR. The pcs_{Aa} gene was amplified by PCR with the primer pair Gibson-PCS-s/Gibson-PCS-as and/or Gibson-PCS-short-s/Gibson-PCS-as, respectively, to generate both the native and truncated pcs_{Aa} sequence.

The verification of the generated DNA fragments for the expected base pair size was carried out by means of gel electrophoresis analysis on a 1% agarose gel and subsequently purified with the NucleoSpin® Gel and PCR Clean-up Kit (Macherey-Nagel, Düren) according to the accompanying protocol. For the construction of the expression plasmids, the plasmid pMKEx2-sts_{Ah}-4cl_{Pc} was linearized with the FastDigest variant (Thermo Fisher Scientific) of the restriction enzymes Nco\ and SeaI. The restriction assay was separated on a 1% agarose gel. The expected fragment of the vector backbone was cleaned from the gel with the NucleoSpin Gel and PCR Clean-up-Kit (Macherey-Nagel, Düren). For the assembly of the DNA fragments by Gibson Assembly (Gibson et al., 2009a), the amplified fragments were individually (pcs_{Aa} or pcs_{Aa}-short) used in threefold molar excess over the linearized vector backbone pMKEx2. The DNA fragments were provided with a prepared Gibson Assembly Master Mix which, in addition to an isothermal reaction buffer, contains the enzymes required for assembly (T5 exonuclease, phusion DNA polymerase and Taq DNA ligase). The assembly of the fragments is carried out at 50° C. for 60 minutes in a thermal cycler. After ligation of the fragments, the total batch volume was used for transformation of chemically competent E. coli DH5oc cells by heat shock at 42° C. for 90 seconds. Following the heat shock, the cells were regenerated on ice for 90 seconds before being provided with 800 μL LB medium and regenerated at 37° C. in a thermal mixer (Eppendorf, Hamburg) at 900 rpm for 60 minutes. Subsequently, 100 μL of the cell suspension was spread on LB agar plates with kanamycin (50 pg/mL) and incubated overnight at 37° C. The correct assembly of the expression plasmids in the grown transformants was checked by means of colony PCR. The 2× DreamTaq Green PCR Master Mix (ThermoFisher Scientific Inc., Waltham, Mass., USA) was used for this purpose. The DNA template was added to the PCR assay by adding cells of the grown colonies. By the initial denaturation step of the PCR protocol at 95° C. for 3 minutes, the cells are lysed so that the DNA template is released and accessible for DNA polymerase. The primer pair chk_pMKEx2_s/chk_pMKEx2_as was used as DNA primer for the colony PCR, which specifically binds to the pMKEx2 vector backbone and, in the case of correct assembly of the fragments used, forms a PCR product of a specific size which was checked by gel electrophoresis. Clones whose PCR product indicates a correct assembly of the expression plasmid construction pMKEx2-pcs_{Aa} and pMKEx2-pcs_{Aa}-short were grown overnight in LB medium with kanamycin (50 pg/mL) for the isolation of the plasmids. The plasmids were then isolated with the NucleoSpin Plasmid (NoLid) Kit (Macherey-Nagel, Düren) and sequenced with said amplification and colony PCR primers.

Primers used:
chk_pMKEx2-s:
CCCTCAAGACCCGTTTAGAGGC
chk_pMKEx2-as:
TTAATACGACTCACTATAGGGGAATTGTGAGC
pMKEx2-pcsAa
Gibson-PCS-s:
ACTTTAAGAAGGAGATATACCATGGTAAGGAGGACAGCTAT-
GTCCTCCTTGTCCAAC
Gibson-PCS-as:
CCAGGACTAGTTTCCAGAGTACTATTACATGAGTGGCAGGGAG
pMKEx2-pcsAa-short
Gibson-PCS-short-s:
ACTTTAAGAAGGAGATATACCATGGTAAGGAGGACAGCTATG-
GAAGATGTGCAGGGC
Gibson-PCS-as:
CCAGGACTAGTTTCCAGAGTACTATTACATGAGTGGCAGGGAG

### Cultivation Conditions

All cultures of C. glutamicum to measure intracellular malonyl-CoA provision, or the synthesis of naringenin, noreugenin and resveratrol, are performed in 50 ml of defined CGXII medium (Keilhauer et al., 1993) with 4% glucose (w/v) in a JRC-1-T incubating shaker (Adolf Kühner AG, Birsfelden, Switzerland) (500 ml, baffled flask, 30° C., 130 rpm). If appropriate, selection antibiotics of the stated concentrations are added:

| Antibiotic | E. coli | C. glutamicum |
|---|---|---|
| Kanamycin (Kan) | 50 μg/ml | 25 μg/ml |
| | | (freely replicating plasmids) |
| | | 15 μg/ml |
| | | (integration of a plasmid into the genome) |
| Spectinomycin (Spec) | 100 μg/ml | 100 μg/ml |

For cultivation in CGXII medium, the strains are first incubated for 6-8 hours in 5 ml BHI medium (brain heart infusion, Difco Laboratories, Detroit, USA) in test tubes at 170 rpm (first preculture) and then used to inoculate 50 ml of CGXII medium in a 500 ml baffled flask (with two opposite baffles). This second preculture is incubated at 30° C. and 130 rpm overnight. The CGXII main culture (50 ml in a 500 ml baffled flask) is inoculated with the grown second preculture to an O D₆₀₀ nm of 1.0 ((malonyl-CoA measurement) and/or 5.0 (production of naringenin, resveratrol or noreugenin), respectively. Optionally, for the synthesis of naringenin and resveratrol, 5 mM of p-cumaric acid (previously dissolved in 80 μl DMSO) is additionally supplemented. The expression of heterologous genes either integrated chromosomally or introduced plasmid-based is induced by the addition of 1 mM isopropyl-ß-D-thiogalactopyranoside (IPTG) 90 minutes after inoculum. At the indicated points in time, 1 ml of culture is collected and stored at −20° C. until use. The product determination (malonyl-CoA or polyphenols or polyketides) is effected as described below. Towards the end of the fermentation, resveratrol, or naringenin or noreugenin, can be further processed optically from the cultivation solution, i.e. separated, purified and/or concentrated.

Determination of the biomass during cultivation to measure malonyl-CoA provision or production of polyphenols or polyketides is carried out by measuring the optical density at a wavelength of 600 nm (OD₆oonm) with the Ultrospec 3300 pro UVA/visible spectrophotometer (Amersham Biosciences, Freiburg). For this purpose, 100 μL of sample volume was taken from the corresponding cultivation and diluted in such a way that the measured OD₆₀oₙm was in the linear measuring range of the photometer of 0.2-0.6. The purification by the dilution factor is used to calculate the actual OD₆₀oₙm of the culture. If a stronger dilution factor of >1:10 (for example 1:100) is to be pipetted, this is carried out sequentially (example: diluted 1:10 twice for a 1:100 dilution).

### Malonyl-CoA Quantification by LC MS/MS

Sample preparation for quantification of the malonyl-CoA intracellular level was performed as previously described (Kallscheuer et al., 2016). 5 mL of the culture is quenched in 15 ml of ice-cold 60% MeOH in H₂O in triplicate and then centrifuged. The malonyl-CoA concentration is determined in the cell extract and in the culture supernatant. In addition, the analysis is carried out in the obtained supernatants after quenching. For the supernatant samples of the culture and after quenching, filtration takes place through 0.2 μm of cellulose acetate filter. Of the culture supernatant, 250 pL is diluted with 750 pL of 60% MeOH; the quenching supernatant was used undiluted.

The quantification of the malonyl-CoA concentration in the samples obtained (cell extract, culture supernatant and quenching supernatant) is carried out by means of LC MS/MS analysis with an Agilent 1260 Infinity HPLC system (Agilent Technologies, Waldbronn, Germany) at 40° C. with a 150*2.1 mm Sequant ZIC-pHILIC-column with 5 pm particle size and a 20*2.1 mm pre-column (Merck, Darmstadt, Germany). Separation is carried out with 10 mM ammonium acetate (pH 9.2) (buffer A) and acetonitrile (buffer B). Before each injection, the column was equilibrated with 90% Buffer B for 15 min. The following gradient is used for separation (injection volume 5 pL): 0 min: 90% B, 1 min: 90% B, 10 min: 70% B, 25 min: 65% B, 35 min: 10% B, 45 min: 10% B, 55 min: 10% B. The measurement is carried out with an ESI-QqTOF-MS (TripleTOF 6600, ab Sciex, Darmstadt, Germany) with an IonDrive ion source. The software Analyst TF 1.7 (AB Sciex, Concord, ON, Canada) is used for data analysis.

As reference, a total of ¹³C-labeled cell extract from Escherichia coli is quantified with ¹³C3 labeled malonyl-CoA in order to obtain a concentration of about 12.5 mM (estimation based on the molecular weight of the free acid). ¹³C3 labeled malonyl-CoA contained [U-¹³C3]malonate as a contamination (data not shown) likely to have resulted from spontaneous hydrolysis of the thioester. This is used as internal standard for malonate quantification and equal volumes of the internal standard solution were added to the samples. Malonate standards with concentrations of 0.01-100 μM in 50% MeOH/H₂O serve as external standard series. A separate external standard series for malonyl-CoA was prepared analogously.

As optimal collision energies for the strongest transitions of malonyl-CoA (852.1>79) and malonate (103>59), −130 eV and −11 eV, respectively, are used. These are determined using the metabolite standards. During the elution, the mentioned transitions and those of the internal standards (855.1>79 and 106>61 respectively) were used for the measurement in the MS/MS high sensitivity mode with the optimum collision energies.

The ¹²C-¹³C isotope ratio was used for the quantification of both metabolites. The standard line was determined by linear regression of isotopic ratios and standard concentrations. To determine the dynamic range, the measurement signals for the highest concentrations were removed so that R² was greater than 0.99. The reduced data set was then log₁₀ transformed to weight lower concentrations in the same way. In the log₁₀ transformed values, measurement signals of the lowest concentrations were discarded so that R² was greater than 0.99.

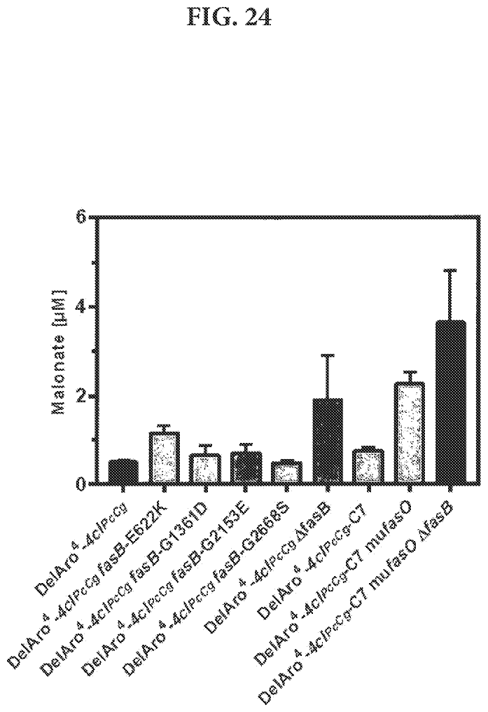

For example, the following malonate (malonyl-CoA) titers are determined using coryneform bacteria cells according to the invention (FIG. 24). The wild-type C. glutamicum ATCC 13032 and/or its derivative of the archetype C. glutamicum DelAro⁴-4c/_{PcCg} has a malonate titer of 0.508 pM under standard conditions. The strains C. glutamicum DelAro⁴-4clp_{cCg} fasB-E622K, DelAro⁴-4c/_{PcCg} fasß-G1361 D, DelAro⁴-4cl_{PcCg} fasß-G2153E and DelAro⁴-4clp_{PcCg} fasB-G2668S have malonate titers of 1.148 pM, 0.658 pM, 0.694 and/or 0.484 pM. The fasB deletion strain DelAro⁴-4cl_{Pc}c_{g} AfasB even yields 1.909 pM malonate. The strain C. glutamicum DelAro⁴-4clp_{cC8}-C7 yields 0.741 pM malonate. The strains C. glutamicum DelAro⁴-4cl_{PcC8}-C7 mufasO and/or C. glutamicum DelAro⁴-4cl_{PcC8}-C7 mufasO AfasB have a titer of 2.261 pM malonate and 3.645 pM malonate, respectively.

### Polyphenol ZPolyketide Quantification by Ethyl Acetate Extraction and LC MS Measurement

The extraction of the products naringenin, noreugenin and resveratrol is carried out as described (Kallscheuer et al., 2016). Samples taken during cultivation were thawed and provided with 1 ml of ethyl acetate and incubated at 1,400 rpm and 20° C. for 10 minutes in an Eppendorf thermal mixer (Hamburg, Germany). The suspension was then centrifuged at 16,000 g for 5 minutes. From the ethyl acetate phase, 800 μl were transferred to a solvent resistant 2 ml deep well plate (Eppendorf, Hamburg, Germany). After evaporation of the solvent overnight, the dried extracts were resuspended in 800 μl of acetonitrile and used directly for LC MS analysis.

The LC-MS analysis of the respective products in the extracts was performed as described with an ultrahigh performance liquid chromatography 1290 Infinity system coupled to a 6130 Quadrupol LC-MS system (Agilent, Waldbronn, Germany) (Kallscheuer et al., 2016). For chromatographic separation, a Kinetex 1.7 μm C18 column with 100 A pore size (50 mm×2.1 mm [internal diameter]), Phenomenex, Torrance, Calif., USA) column was used at 50° C. As mobile phases, 0.1% acetic acid (Phase A) and acetonitrile+0.1% acetic acid (Phase B) were used at a flow rate of 0.5 ml/min. This was followed by a gradient elution in which the proportion of phase B was increased stepwise: Minute 0-6: 10-30%, minute 6-7: 30-50%, minute 7-8: 50-100%, minute 8-8.5: 100-10%. The mass spectrometer was operated in negative electrospray ionization mode (ESI); the data acquisition was in Selected Ion Monitoring Mode (SIM). For the quantification, pure product standards of various concentrations were prepared in acetonitrile. The measured planes for the [M-H]- mass signals (m/z 271 for Naringenin, m/z 191 for Noreugenin, m/z 227 for Resveratrol) were linear for concentrations up to 250 mg/l. Benzoate (final concentration 100 mg/l, m/z 121 for benzoate) was used as internal standard. A calibration curve was calculated based on the ratio of the measured surfaces of the analyte to internal standard.

With the coryneform bacteria cells according to the invention, the following polyphenol or polyketide titers are determined, in each case under standard conditions when growing on glucose or glucose supplemented with p-cumaric acid. The wild-type C. glutamicum ATCC 13032 and its derivative, the archetype C. glutamicum DelAro⁴-4c/_{PcCg} pMKEx2-stsAh-4clPc have a resveratrol titer of 8 mg/L and 12 mg/L, respectively, under standard conditions. The strains C. glutamicum DelAro⁴-4c/p_{cCg} fasB-E622K pMKEx2-stsAh-4clPc, DelAro⁴-4c/p_{cCg} fasß-G1361 D pMKEx2-stsAh-4clPc, DelAro⁴-4c/p_{cCg} fasB-G2153E pMKEx2-stsAh-4clPc and DelAro⁴-4c/_{PcCg} fasB-G2668S pMKEx2-stsAh-4clPc have a resveratrol titer of 9 mg/L and/or 28.90 mg/L, 8.37 mg/L bzw. 18.20 mg/L, 8.49 mg/L and/or 20.30 mg/L and 7.89 mg/L and/or 11.70 mg/L resveratrol. The fasB deletion strain DelAro⁴-4cl_{PcCg} AfasB pMKEx2-stsAh-4clPc reaches even 9.49 mg/L and/or 37 mg/L resveratrol, respectively. With the strain C. glutamicum DelAro⁴-4cl_{PcC8}-C7 pMKEx2-stsAh-4clPc, 14 mg/L and/or 113 mg/L resveratrol are achieved. The strains C. glutamicum DelAro⁴-4cl_{PcCg}-C7-mufasO pMKEx2-stsAh-4clPc and/or C. glutamicum DelAro⁴-4cl_{Pc}c_{g}-C7-mufasO-AfasB pMKEx2-stsAh-4clPc have a titer of 22.85 mg/L and/or 262 mg/L resveratrol and 22.73 mg/L and 260 mg/L resveratrol.

With respect to naringenin production, the coryneform bacteria cells according to the invention have the following titers, in each case under standard conditions when grown on glucose or glucose supplemented with p-cumaric acid. The wild-type C. glutamicum ATCC 13032 and/or its derivative the archetype C. glutamicum DelAro⁴-4c/p_{cCg} pMKEx2-chsPh-chiPh has a naringenin titer of 1 mg/L and/or 2.1 mg/L under standard conditions. The strains C. glutamicum DelAro⁴-4c/_{PcCg} fasß-E622K pMKEx2-chsPh-chiPh, DelAro⁴-4c/p_{cCg} fasß-G1361 D pMKEx2-chsPh-chiPh, DelAro⁴-4cl_{PcCg} fasB-G2153E pMKEx2-chsPh-chiPh and DelAro⁴-4cl_{PcC8} fasß-G2668S pMKEx2-chsPh-chiPh have naringenin titers of 1.78 mg/L and/or 7.11 mg/L, 1.32 mg/L and/or 4.54 mg/L, 1.55 mg/L and/or 5.08 mg/L and 1.16 mg/L and/or 2.84 mg/L naringenin. The fasB deletion strain DelAro⁴-4cl_{PcCg} AfasB pMKEx2-chsPh-chiPh reaches even 2.15 mg/L and/or 9.61 mg/L of naringenin. With the strain C. glutamicum DelAro⁴-4cl_{PcC8}-C7 pMKEx2-chsPh-chiPh, 3.5 mg/L and/or 18.5 mg/L resveratrol are achieved. The strains C. glutamicum DelAro⁴-4cl_{Pe}c_{g}-C7-mufasO pMKEx2-chsPh-chiPh and C. glutamicum DelAro⁴-4cl_{PcC8}-C7-mufasO-AfasB pMKEx2-chsPh-chiPh have a titer of 10.59 mg/L and 65 mg/L naringenin and 9.83 mg/L and 60 mg/L naringenin, respectively.

The coryneform bacteria cells according to the invention have the following noreugenin titers under standard conditions when grown on glucose. No noreugenin (0.002 mg/L) could be detected for the wild-type C. glutamicum ATCC 13032 pMKEx2-pcs_{Aacg-short} and/or its derivative, the archetype C. glutamicum DelAro⁴-4clp_{cCg} pMKEx2-pcs_{AaC8-short}. The strains C. glutamicum DelAro⁴-4c/p_{cCg} fasß-E622K pMKEx2-pcs_{AaCg-Short}, DelAro⁴-4c/_{PcCg} fasß-G1361 D pMKEx2-pcs_{Aacg-short}, DelAro⁴-4c/p_{cCg} fasB-G2153E pMKEx2-pcs_{AaC8-short} and DelAro⁴-4c/p_{cCg} fasB-G2668S pMKEx2-pcs_{Aac8-short} have noreugenin titer of 0.004 mg/L, 0.003 mg/L, 0.003 mg/L and 0.003 mg/L noreugenin. The strain C. glutamicum DelAro⁴-4cl_{Pc}c_{g}-C7 pMKEx2-pcs_{Aacg-short} results in 0.86 mg/L noreugenin that is determined. The strain C. glutamicum DelAro⁴-4cl_{PcC8}-C7-mufasO pMKEx2-pcs_{AaCg.Short} has a titer of 4.4 mg/L noreugenin. The strain C. glutamicum DelAro⁴-4cl_{PcC8}-C7-mufasO-AfasB pMKEx2-pcs_{AaCg-short} has a titer of 4.51 mg/L noreugenin.

TABLE 1

| Strain | Description | Reference |
|---|---|---|
| C. glutamicum ATCC | Wild type | Abe et al., 1967 |
| 13032 | | (https://doi.org/10.2323/ |
| | | jgam.13.279) |
| C. glutamicum MB001 | Prophage free variant of wild type | Kortmann, M. et al., 2015. |
| (DE3) | ATCC 13032 with chromosomally | https://doi.org/10.1111/ |
| | coded T7 gene 1 (cg1122-Placl-lacl- | 1751-7915.12236 |
| | PlacUV5-lacZα-T7 gene 1-cg1121) | |
| C. glutamicum DelAro³ | C. glutamicum MB001 (DE3) | Kallscheuer, N. et al.; 2016. |
| | derivative with in-frame deletion of | https://doi.org/10.1016/ |
| | cg0344-47, cg2625-40 and cg1226 | j.ymben.2016.06.003 |
| C. glutamicum DelAro⁴ | C. glutamicum DelAro³ derivative with | Kallscheuer, N. et al.; 2016. |
| | in-frame deletion of cg0502 | https://doi.org/10.1016/ |
| | | j.ymben.2016.06.003 |
| C. glutamicum DelAro⁴ | C. glutamicum DelAro⁴ derivative with | herein |
| fesB-E622K | fasB variant with amino acid | |
| | substitution E622K | |
| C. glutamicum DelAro⁴ | C. glutamicum DelAro⁴ derivative with | herein |
| fesB-G1361D | fasB variant with amino acid | |
| | substitution G1361D | |
| C. glutamicum DelAro⁴ | C. glutamicum DelAro⁴ derivative with | herein |
| fesB-G2153D | fasB variant with amino acid | |
| | substitution G2153D | |
| C. glutamicum DelAro⁴ | C. glutamicum DelAro⁴ derivative with | herein |
| fesB-G2668S | fasB variant with amino acid | |
| | substitution G2668S | |
| C. glutamicum DelAro⁴ | C. glutamicum DelAro⁴ derivative with | herein |
| ΔfasB | in-frame deletion of fasB (cg2743) | |
| C. glutamicum DelAro⁴- | C. glutamicum DelAro⁴ derivative with | herein |
| C7 | replacement of the native gltA | |
| | promoter against the dapA promoter | |
| | variant C7 (P_{gl-tA}::P_{dapA}-C7) | |
| C. glutamicum DelAro⁴- | C. glutamicum DelAro⁴-C7 derivative | herein |
| C7 ΔfasB | with in-frame deletion of fasB | |
| | (cg2743) | |
| C. glutamicum DelAro⁴- | C. glutamicum DelAro⁴-C7 derivative | herein |
| C7 muufasO | with mutation of the fasO binding site | |
| | upstream of the genes accBC | |
| | (cg0802) and accD1 (cg0812) | |
| C. glutamicum DelAro⁴- | C. glutamicum DelAro⁴-C7 mufasO | herein |
| C7 mu/asO ΔfasB | derivative with in-frame deletion of | |
| | fasB (cg2743) | |
| C. glutamicum DelAro³- | C. glutamicum DelAro³ derivative with | Kallscheuer, N. et al; 2016. |
| 4cl_{Pc} | chromosomal integration of the gene | https://doi.org/10.1016/ |
| | 4cl from Petroselinum crispum, | j.ymben.2016.06.003 |
| | codon-optimized for C. glutamicum, | |
| | under control of the IPTG-inducible | |
| | T7 promoter to the deletion locus | |
| | Dcg0344-47 (Dcg0344-47::P_{T7}- | |
| | 4cl_{PcCg}) | |
| C. glutamicum DelAro⁴- | C. glutamicum DelAro⁴ derivative with | Kallscheuer, N. et al; 2016. |
| 4cl_{Pc} | integration of the gene 4cl from | https://doi.org/10.1016/ |
| | Petroselinum crispum, codon- | j.ymben.2016.06.003 |
| | optimized for C. glutamicum, under | |
| | control of the IPTG-inducible T7 | |
| | promoter to the deletion locus | |
| | Dcg0344-47 (Dcg0344-47::P_{T7}- | |
| | 4cl_{PcCg}) | |
| C. glutamicum DelAro⁴- | C. glutamicum DelAro⁴ fasB-E622K | herein |
| 4cl_{pc} fasB-E622K | derivative with integration of the gene | |
| | 4cl from Petroselinum crispum, | |
| | codon-optimized for C. glutamicum, | |
| | under control of the IPTG-inducible | |
| | T7 promoter to the deletion locus | |
| | Dcg0344-47 (Dcg0344-47::P_{T7}- | |
| | 4cl_{PcCg}) | |
| C. glutamicum DelAro⁴- | C. glutamicum DelAro⁴ fasB-G1361D | herein |
| 4cl_{Pc} fasB-G1361D | derivative with integration of the gene | |
| | 4cl from Petroselinum crispum, | |
| | codon-optimized for C. glutamicum, | |
| | under control of the IPTG-inducible | |
| | T7 promoter to the deletion locus | |
| | Dcg0344-47 (Dcg0344-47::P_{T7}- | |
| | 4cl_{PcCg}) | |
| C. glutamicum DelAro⁴- | C. glutamicum DelAro⁴ fasB-G2153D | herein |
| 4cl_{Pc} fasB-G2153D | derivative with integration of the gene | |
| | 4cl from Petroselinum crispum, | |
| | codon-optimized for C. glutamicum, | |
| | under control of the IPTG-inducible | |
| | T7 promoter to the deletion locus | |
| | Dcg0344-47 (Dcg0344-47::P_{T7}- | |
| | 4cl_{PcCg}) | |
| C. glutamicum DelAro⁴- | C. glutamicum DelAro⁴ fasB-G2668S | herein |
| 4cl_{Pc} fasB-G2668S | derivative with integration of the gene | |
| | 4cl from Petroselinum crispum, | |
| | codon-optimized for C. glutamicum, | |
| | under control of the IPTG-inducible | |
| | T7 promoter to the deletion locus | |
| | Dcg0344-47 (Dcg0344-47::P_{T7}- | |
| | 4cl_{PcCg}) | |
| C. glutamicum DelAro⁴- | C. glutamicum DelAro⁴ DfasB | herein |
| 4cl_{Pc} ΔfasB | derivative with integration of the gene | |
| | 4cl from Petroselinum crispum, | |
| | codon-optimized for C. glutamicum, | |
| | under control of the IPTG-inducible | |
| | T7 promoter to the deletion locus | |
| | Acg0344-47 (Δcg0344-47::P_{T7}- | |
| | 4cl_{PcCg}) | |
| C. glutamicum DelAro⁴- | C. glutamicum DelAro⁴-C7 derivative | herein |
| 4cl_{Pc}-C7 | with integration of the gene 4cl from | |
| | Petroselinum crispum, codon- | |
| | optimized for C. glutamicum, under | |
| | control of the IPTG-inducible T7 | |
| | promoter to the deletion locus | |
| | Δcg0344-47 (Δcg0344-47::P_{T7}- | |
| | 4cl_{PcCg}) | |
| C. glutamicum DelAro⁴- | C. glutamicum DelAro⁴-C7 ΔfasB | herein |
| 4cl_{Pc}-C7 ΔfasB | derivative with integration of the gene | |
| | 4cl from Petroselinum crispum, | |
| | codon-optimized for C. glutamicum, | |
| | under control of the IPTG-inducible | |
| | T7 promoter to the deletion locus | |
| | Δcg0344-47 (Δcg0344-47::PT7- | |
| | 4cl_{PcCg}) | |
| C. glutamicum DelAro⁴- | C. glutamicum DelAro⁴-C7 mufasO | herein |
| 4cl_{PC}-C7 mufasO | derivative with integration of the gene | |
| | 4cl from Petroselinum crispum, | |
| | codon-optimized for C. glutamicum, | |
| | under control of the IPTG-inducible | |
| | T7 promoter to the deletion locus | |
| | Δcg0344-47 (Δcg0344-47::P_{T7}- | |
| | 4cl_{PcCg}) | |
| C. glutamicum DelAro⁴- | C. glutamicum DelAro⁴-C7 mufasO | herein |
| 4cl_{Pc}-C7 mu/asO ΔfasB | ΔfasB derivative with integration of | |
| | the gene 4cl from Petroselinum crispum, | |
| | codon-optimized for C. glutamicum, | |
| | under control of the | |
| | IPTG-inducible T7 promoter to the | |
| | deletion locus Δcg0344-47 | |
| | (Dcg0344-47:: P_{T7}-4cl_{PcCg}) | |
| E. coli DH5 α | F- Φ80IacZΔM15 Δ(lacZYA- | Thermo Fisher Scientific |
| | argF)U169 recA1 endA1hsdR17 (rK-, | (Waltham, MA, USA) |
| | mKp) phoA supE44 λ- thi-1 gyrA96 | |
| | relA1 | |

TABLE 2

| Plasmid | Description | Reference |
|---|---|---|
| pK19mobsacB | Vector for allelic exchange | Schafer, A. et al.; 1994. |
| | in C. glutamicum (pK18, | “Small mobilizable |
| | Kanamycin^{R}, oriV_{Ec}, sacB, IacZa) | multi-purpose cloning |
| | | vectors derived from |
| | | the Escherichia coli |
| | | plasmids pK18 and |
| | | pK19: selection of |
| | | defined deletions in the |
| | | chromosome of |
| | | Corynebacterium |
| | | glutamicum.” Gene, |
| | | 145 (1) |
| pK19mobsacB- | PK19mobsacB-based vector for | Kallscheuer, N. et al.; 2016. |
| cg0344-47-del | in-frame deletion of the | https://doi.org/10.1016/ |
| | genes cg0344-47 | j.ymben.2016.06.003 |
| pK19mobsacB- | PK19mobsacB-based vector for | Kallscheuer, N. et al.; 2016. |
| cg2625-40-del | in-frame deletion of the | https://doi.org/10.1016/ |
| | genes cg2625-40 | j.ymben.2016.06.003 |
| pK19mobsacB- | PK19mobsacB-based vector for | Kallscheuer, N. et al.; 2016. |
| cg1226-del | in-frame deletion of the | https://doi.org/10.1016/ |
| | gene cg1226 | j.ymben.2016.06.003 |
| pK19mobsacB- | PK19mobsacB-based vector for | Kallscheuer, N. et al.; 2016. |
| cg0502-del | in-frame deletion of the | https://doi.org/10.1016/ |
| | gene cg0502 | j.ymben.2016.06.003 |
| pK19mobsacB-fasB- | PK19mobsacB-based vector for | herein |
| E622K | amino acid substitution E622K | |
| | in the fasB gene (cg2743) | |
| pK19mobsacB-fasB- | PK19mobsacB-based vector for | herein |
| G1361D | amino acid substitution G1361D | |
| | in the fasB gene (cg2743) | |
| pK19mobsacB-fasB- | PK19mobsacB-based vector for | herein |
| G2153D | amino acid substitution G2153D | |
| | in the fasB gene (cg2743) | |
| pK19mobsacB-fasB- | PK19mobsacB-based vector for | herein |
| G2668S | amino acid substitution G2668S | |
| | in the fasB gene (cg2743) | |
| pK19mobsacB- | PK19mobsacB-based vector for | herein |
| ΔfasB | in-frame deletion of fasB | |
| | (cg2743) | |
| pK19mobsacB-gItA- | PK19mobsacB-based vector for | van Ooyen, J. et al.; 2012. |
| C7 | replacing the native promoter | https://doi.org/10.1002/bit.24486 |
| | of gltA with the C7 variant of | |
| | the dapA promoter (P_{gltA}::P_{dapA}-C7) | |
| pK19mobsacB- | PK19mobsacB-based vector for | herein |
| mu/asO-accBC | mutation of the fasO binding | |
| | site upstream of accBC (cg0802) | |
| pK19mobsacB- | PK19mobsacB-based vector for | herein |
| mufasO-accD1 | mutation of the fasO binding | |
| | site upstream of accD1 (cg0812) | |
| | taking into account the ATG start | |
| | codon and the amino acid | |
| | sequence of accD1 | |
| pK19mobsacB- | pK19mobsacB-based vector for | Kallscheuer, N. et al.; |
| Δcg0344-47::P_{T7}- | the chromosomal integration of | 2016. |
| 4cl_{Pc} | the gene 4cl from Petroselinum crispum, | https://doi.org/10.1016/ |
| | codon-optimized for C. glutamicum, | j.ymben.2016.06.003 |
| | under control of the | |
| | IPTG-inducible T7 promoter to the deletion | |
| | locus Dcg0344-47 (Dcg0344-47::P_{T7}-4cl_{PcCg})- | |
| pMKEx2 | E. coli/C. glutamicum Shuttle vector | Kortmann, M. et al.; 2015. |
| | (Kanamycin^{R}, lacl, P_{T7}, lacO1, pHM1519 | https://doi.org/10.1111/ |
| | ori_{Cg} pACYC177 ori_{Ec}) | 1751-7915.12236 |
| pM KEx2-stS_{Ah}-4cl_{Pc} | pMKEx2 derivative for the expression | Kallscheuer, N. et al.; 2016. |
| | of the codon-optimized genes for | https://doi.org/10.1016/ |
| | C. glutamicum for a stilbene synthase | j.ymben.2016.06.003 |
| | (sts) from Arachis hypogea and a 4- | |
| | coumarate-CoA ligase (4cl) from | |
| | Petroselinum crispum under control of the | |
| | IPTG- inducible T7 promoter. | |
| pMKEx2-chS_{Ph}-chi_{Ph} | pMKEx2 derivative for the expression of | Kallscheuer, N. et al.; 2016. |
| | the codon-optimized genes for C. glutamicum | https://doi.org/10.1016/ |
| | for a chaicone synthase (chs) from | j.ymben.2016.06.003 |
| | Petunia x hybrida and a chaicone isomerase | |
| | (chi) from Petunia x hybrida under control | |
| | of the IPTG inducible T7 promoter | |
| pMKEx2-pcs_{Aa} | PMKEx2 derivative for expressing the | herein |
| | codon-optimized gene for C. glutamicum, | |
| | a pentaketide chromone synthase (pcs) | |
| | from Aloe arborescens | |
| pMKEx2-pcs_{Aa}-short | PMKEx2 derivative for the expression | herein |
| | of a truncated variant of the codon- | |
| | optimized gene for C. glutamicum for | |
| | a pentaketide chromone synthase (pcs) | |
| | from Aloe arborescens | |
| pEKEx3 | E. coli/C. glutamicum shuttle vector | Gande, R. et al.; 2007. |
| | (Spectinomy-cin^{R}, lacl, P_{tac}, lacO1, | https://doi.org/10.1128/ |
| | pBL1 ori_{Cg}, pUC ori_{Ec}) | JB.00254-07 |
| pEKEx3-aroH_{Ec}-tal_{Fj} | PEKEx3 derivative for the expression of | Kallscheuer, N. et al.; 2016. |
| | the native gene for a 3-deoxy-D-arabino- | https://doi.org/10.1016/ |
| | heptulosonate-7-phosphate synthase (aroH) | j.ymben.2016.06.003 |
| | from E. coli and for a codon-optimized | |
| | gene for C. glutamicum for a tyrosine | |
| | ammonia lyase (tal) from Flavobaderium johnsoniae | |
| | under the control of the IPTG-inducible tac promoter | |

TABLE 3

| Sequence | Description | Reference |
|---|---|---|
| SEQ ID NO. 1 | Nucleic acid sequence of | herein |
| | the coding region of the | |
| | fatty acid synthase gene | |
| | fasB from coryneform bacteria | |
| | with a nucleotide substitution | |
| | at position 1864 (g −> a) | |
| SEQ ID NO. 2 | Amino acid sequence of coryneform bacteria for | herein |
| | homologous fatty acid synthase with reduced | |
| | activity with amino acid exchange at position | |
| | 622 (E −> K) | |
| SEQ ID NO. 3 | Nucleic acid sequence of the coding region of | herein |
| | the fatty acid synthase gene fasB from | |
| | coryneform bacteria with a nucleotide | |
| | substitution at position 4082 (g −> a) | |
| SEQ ID NO. 4 | Amino acid sequence from coryneform bacteria | herein |
| | for a homologous fatty acid synthase with | |
| | reduced activity with an amino acid | |
| | replacement at position 1361 (G −> D) | |
| SEQ ID NO. 5 | Nucleic acid sequence of the coding region of | herein |
| | the fatty acid synthase gene fasB from | |
| | coryneform bacteria with a nucleotide | |
| | substitution at position 6458 (g −> a) | |
| SEQ ID NO. 6 | Amino acid sequence of coryneform bacteria for | herein |
| | homologous fatty acid synthase with reduced | |
| | activity with amino acid exchange at position | |
| | 2153 (G −> E) | |
| SEQ ID NO. 7 | Nucleic acid sequence of the coding region of | herein |
| | the fatty acid synthase gene fasB from | |
| | coryneform bacteria with nucleotide | |
| | substitutions at positions 8002-8004 (ggt −> tcc) | |
| SEQ ID NO. 8 | Amino acid sequence of coryneform bacteria for | herein |
| | homologous fatty acid synthase with reduced | |
| | activity with amino acid replacement at position | |
| | 2668 (G −> S) | |
| SEQ ID NO. 9 | Nucleic acid sequence of coryneform bacteria | herein |
| | with nucleotide deletions at positions 25-8943 | |
| | (ΔfasB) of the coding region of the fatty acid | |
| | synthase gene fasB | |
| SEQ ID NO. 10 | Amino acid sequence of coryneform bacteria for | herein |
| | homologous fatty acid synthase with switched- | |
| | off activity by amino acid deletions over most of | |
| | the encoded protein (ΔFasB) | |
| SEQ ID NO. 11 | Nucleic acid sequence P_{gltA}:: Pda_{dapA}-C7 with | herein |
| | nucleotide substitutions in the 5′ operatively | |
| | linked promoter region of the gltA gene | |
| | encoding the citrate synthase from coryneform | |
| | bacteria, wherein the promoter P_{gltA} is | |
| | exchanged with the promoter of the dapA gene | |
| | (P_{dapA}) from coryneform bacteria and its | |
| | functionality is reduced by means of nucleotide | |
| | substitutions; (P_{gltA}::Pda_{daypA}-C7 substitution at | |
| | position 95 (a −> t) and 96 (g −> a) in the 5′ | |
| | regulatory region (1-265) upstream from the | |
| | start codon ATG of gtlA) | |
| SEQ ID NO. 12 | Nucleic acid sequence of the native promoter | Vasicova et al, 1999; |
| | region P_{dapA} (position 1-265 before the start | PMID 10498736 |
| | codon ATG of gtlA) from Corynebacterium | |
| | glutamicum wild-type ATCC13032 | |
| SEQ ID NO. 13 | Nucleic acid sequence with one or more | Nickel et al; 2010; |
| | nucleotide substitutions in the fasO binding site | https://doi.org/10.1111/ |
| | of the accBC gene; (5′ - regulatory region | j.1365-2958.2010.07337.x |
| | mufasO-accBC with substitutions at positions | |
| | 11-13 (tga −> gtc) and 20-22 (cct −> aag)) | |
| SEQ ID NO. 14 | Nucleic acid sequence of the operatively | Nickel et al; 2010; |
| | linked FasO binding site of the accBC gene | https://doi.org/10.1111/ |
| | from Corynebacterium glutamicum wild- type | j.1365-2958.2010.07337.x |
| | ATCC 13032 | |
| SEQ ID NO. 15 | Nucleic acid sequence with one or more | herein |
| | nucleotide substitutions in the operatively linked | |
| | fasO binding site of the accD1 gene; (5′ | |
| | regulatory region mufasO-accD1 with | |
| | substitutions at positions 20-24 (cctca −> gtacg)) | |
| SEQ ID NO. 16 | Nucleic acid sequence of the operatively | Nickel et al; 2010; |
| | linked fasO binding site of the accD1 gene | https://doi.org/10.1111/ |
| | from Corynebacterium glutamicum wild-type | j.1365-2958.2010.07337.x |
| | ATCC 13032 | |
| SEQ ID NO. 17 | Nucleic Acid Sequence of the gene pcs_{Aa} | Abe et al.; 2005; |
| | coding a pentaketide chromone synthase from | https://doi.org/10.1021/ |
| | the Aloe Arborescens wild-type | ja0431206 |
| SEQ ID NO. 18 | Amino acid sequence of the pentaketide | Abe et al.; 2005; |
| | chromone synthase (PCS_{Aa}) from the Aloe | https://doi.org/10.1021/ |
| | arborescens wild-type | ja0431206 |
| SEQ ID NO. 19 | Nucleic acid sequence of the gene pcs_{AaCg-short} | herein |
| | for the expression of the gene variant of pcs | |
| | from Aloe arborescens (pcs_{Aa}) with adaptation | |
| | to codon usage of C. glutamicum in coryneform | |
| | bacteria cells. | |
| SEQ ID NO. 20 | Amino acid sequence of the variant of the | herein |
| | pentaketide chromone synthase PCS_{AaCg-short} | |
| | from aloe arborescens for expression in | |
| | coryneform bacteria cells | |
| SEQ ID NO. 21 | Nucleic acid sequence of the gene 4cl_{PcCg} from | Kallscheuer, N. et al.; 2016; |
| | Petroselinum crispum with adaptation to codon | https://doi.org/10.1016/ |
| | usage of C. glutamicum for expression in | j.ymben.2016.06.003 |
| | coryneform bacteria cells | |
| SEQ ID NO. 22 | Amino acid sequence encoding 4-cumorate | Kallscheuer, N. et al.; 2016; |
| | CoA ligase (4cl) from Petroselinum crispum for | https://doi.org/10.1016/ |
| | expression in coryneform bacteria cells | j.ymben.2016.06.003 |
| SEQ ID NO. 23 | Nucleic acid sequence of the gene sts_{AhCg} | Kallscheuer, N. et al.; 2016; |
| | produced from Arachis hypogea with | https://doi.org/10.1016/ |
| | adaptation to codon usage of C. glutamicum for | j.ymben.2016.06.003 |
| | expression in coryneform bacteria cells | |
| SEQ ID NO. 24 | Amino acid sequence encoding stilbene | Kallscheuer, N. et al.; 2016; |
| | synthase (STS) from Arachis hypogea for | https://doi.org/10.1016/ |
| | expression in coryneform bacteria cells | j.ymben.2016.06.003 |
| SEQ ID NO. 25 | Nucleic acid sequence of the gene chs_{PhCg}from | Kallscheuer, N. et al.; 2016; |
| | Petunia x hybrida with adaptation to codon | https://doi.org/10.1016/ |
| | usage of C. glutamicum for expression in | j.ymben.2016.06.003 |
| | coryneform bacteria cells | |
| SEQ ID NO. 26 | Amino acid sequence encoding chaicone | Kallscheuer, N. et al.; 2016; |
| | synthase (CHS) from Petunia x hybrida for | https://doi.org/10.1016/ |
| | expression in coryneform bacteria cells | j.ymben.2016.06.003 |
| SEQ ID NO. 27 | Nucleic acid sequence of the gene chi_{PhCg}from | Kallscheuer, N. et al.; 2016; |
| | Petunia x hybrida with adaptation to codon | https://doi.org/10.1016/ |
| | usage of C. glutamicum for expression in | j.ymben.2016.06.003 |
| | coryneform bacteria cells | |
| SEQ ID NO. 28 | Amino acid sequence encoding chaicone | Kallscheuer, N. et al.; 2016; |
| | isomerase (CHI) from Petunia x hybrida for | https://doi.org/10.1016/ |
| | expression in coryneform bacteria cells | j.ymben.2016.06.003 |
| SEQ ID NO. 29 | Nucleic Acid Sequence of the gene aroH_{Ec} from | Kallscheuer, N. et al.; 2016; |
| | Escherichia coli for Expression in coryneform | https://doi.org/10.1016/ |
| | bacteria cells | j.ymben.2016.06.003 |
| SEQ ID NO. 30 | Amino acid sequence coding a feedback | Kallscheuer, N. et al.; 2016; |
| | resistant 3-deoxy-D-arabino-heptulosonate-7- | https://doi.org/10.1016/ |
| | phosphate synthase (/XroH) from E. coli for | j.ymben.2016.06.003 |
| | expression in coryneform bacteria cells | |
| SEQ ID NO. 31 | Nucleic acid sequence of the gene tal_{FjCg} from | Kallscheuer, N. et al.; 2016; |
| | Flavobacterium johnsoniae with adaptation to | https://doi.org/10.1016/ |
| | codon usage of C. glutamicum forexpression in | j.ymben.2016.06.003 |
| | coryneform bacteria cells | |
| SEQ ID NO. 32 | Amino acid sequence coding a tyrosine | Kallscheuer, N. et al.; 2016; |
| | ammonium lyase (tai) from Flavobacterium | https://doi.org/10.1016/ |
| | johnsoniae (tal_{Fj}) for expression in coryneform | j.ymben.2016.06.003 |
| | bacteria cells | |
| SEQ ID NO. 33 | Primer PgltA-up-s | herein |
| SEQ ID NO. 34 | Primer PgltA-up-as | herein |
| SEQ ID NO. 35 | Primer PgltA-down-s | herein |
| SEQ ID NO. 36 | Primer PgltA-down-as | herein |
| SEQ ID NO. 37 | Primer PdapA-s | herein |
| SEQ ID NO. 38 | Primer PdapA-as | herein |
| SEQ ID NO. 39 | Primer chk-PgltA-s | herein |
| SEQ ID NO. 40 | Primer chk-PgltA-as | herein |
| SEQ ID NO. 41 | Primer univ | herein |
| SEQ ID NO. 42 | Primer rsp | herein |
| SEQ ID NO. 43 | Primer mu-accBC-up-s | herein |
| SEQ ID NO. 44 | Primer mu-accBC-up-as | herein |
| SEQ ID NO. 45 | Primer mu-accBC-down-s | herein |
| SEQ ID NO. 46 | Primer mu-accBC-down-as | herein |
| SEQ ID NO. 47 | Primer chk-accBC-s | herein |
| SEQ ID NO. 48 | Primer chk-accBC-as | herein |
| SEQ ID NO. 49 | Primer mu-accD1-up-s | herein |
| SEQ ID NO. 50 | Primer mu-accD1-up-as | herein |
| SEQ ID NO. 51 | Primer mu-accD1-down-s | herein |
| SEQ ID NO. 52 | Primer mu-accD1-down-as | herein |
| SEQ ID NO. 53 | Primer chk-accD1-s | herein |
| SEQ ID NO. 54 | Primer chk-accDT\hat{ }as | herein |
| SEQ ID NO. 55 | Primer fasB-(cg2743)-up-s | herein |
| SEQ ID NO. 56 | Primer fasB-(cg2743)-up-as | herein |
| SEQ ID NO. 57 | Primer fasB-(cg2743)-down-s | herein |
| SEQ ID NO. 58 | Primer fasB-(cg2743)-down-as | herein |
| SEQ ID NO. 59 | Primer chk-fasB-s | herein |
| SEQ ID NO. 60 | Primer chk-fasB-as | herein |
| SEQ ID NO. 61 | Primer OL_622-s | herein |
| SEQ ID NO. 62 | Primer OL_622-as | herein |
| SEQ ID NO. 63 | Primer Sbfl_622-s | herein |
| SEQ ID NO. 64 | Primer Xbal_622-as | herein |
| SEQ ID NO. 65 | Primer OL_1361-s | herein |
| SEQ ID NO. 66 | Primer OL_1361-as | herein |
| SEQ ID NO. 67 | Primer Sbfl_1361-s | herein |
| SEQ ID NO. 68 | Primer Xbal_1361-as | herein |
| SEQ ID NO. 69 | Primer OL_2153-s | herein |
| SEQ ID NO. 70 | Primer OL_2153-as | herein |
| SEQ ID NO. 71 | Primer Sbfl_2153-s | herein |
| SEQ ID NO. 72 | Primer Xbal_2153-as | herein |
| SEQ ID NO. 73 | Primer OL_G2668S-s | herein |
| SEQ ID NO. 74 | Primer OL_G2668S-as | herein |
| SEQ ID NO. 75 | Primer Sbfl_G2668S-s | herein |
| SEQ ID NO. 76 | Primer Xbal_G2668S-as | herein |
| SEQ ID NO. 77 | Primer cg0344-47-up-s | herein |
| SEQ ID NO. 78 | Primer cg0344-47-up-as | herein |
| SEQ ID NO. 79 | Primer cg0344-47-down-s | herein |
| SEQ ID NO. 80 | Primer cg0344-47-down-as | herein |
| SEQ ID NO. 81 | Primer del-cg0344-47-s | herein |
| SEQ ID NO. 82 | Primer del-cg0344-47-as | herein |
| SEQ ID NO. 83 | Primer cg2625-40-up-s | herein |
| SEQ ID NO. 84 | Primer cg2625-40-up-as | herein |
| SEQ ID NO. 85 | Primer cg2625-40-down-s | herein |
| SEQ ID NO. 86 | Primer cg2625-40-down-as | herein |
| SEQ ID NO. 87 | Primer del-cg2625-40-s | herein |
| SEQ ID NO. 88 | Primer del-cg2625-40-as | herein |
| SEQ ID NO. 89 | Primer Mlul-PT7-4CLPcCg-s | herein |
| SEQ ID NO. 90 | Primer Ndel-4CLPcCg-as | herein |
| SEQ ID NO. 91 | Primer up-cg0502-s | herein |
| SEQ ID NO. 92 | Primer up-cg0502-as | herein |
| SEQ ID NO. 93 | Primer down-cg0502-s | herein |
| SEQ ID NO. 94 | Primer down-cg0502-as | herein |
| SEQ ID NO. 95 | Primer del-cg0502-s | herein |
| SEQ ID NO. 96 | Primer del-cg0502-as | herein |
| SEQ ID NO. 97 | Primer up-cg1226-s | herein |
| SEQ ID NO. 98 | Primer up-cg1226-as | herein |
| SEQ ID NO. 99 | Primer down-cg1226-s | herein |
| SEQ ID NO. 100 | Primer down-cg1226-as | herein |
| SEQ ID NO. 101 | Primer del-cg01226-s | herein |
| SEQ ID NO. 102 | Primer del-cg1226-as | herein |
| SEQ ID NO. 103 | Primer aroHEc-s | herein |
| SEQ ID NO. 104 | Primer aroHEc-as | herein |
| SEQ ID NO. 105 | Primer talFj-s | herein |
| SEQ ID NO. 106 | Primer talFj-as | herein |
| SEQ ID NO. 107 | Primer chk_pEKEx3_s | herein |
| SEQ ID NO. 108 | Primer chk_pEKEx3_as | herein |
| SEQ ID NO. 109 | Primer stsAh-s | herein |
| SEQ ID NO. 110 | Primer stsAh-as | herein |
| SEQ ID NO. 111 | Primer 4clPc-s | herein |
| SEQ ID NO. 112 | Primer 4clPc-as | herein |
| SEQ ID NO. 113 | Primer chk_pMKEx2-s | herein |
| SEQ ID NO. 114 | Primer chk_pMKEx2-as | herein |
| SEQ ID NO. 115 | Primer chsPh-s | herein |
| SEQ ID NO. 116 | Primer chsPh-as | herein |
| SEQ ID NO. 117 | Primer chiPh-s | herein |
| SEQ ID NO. 118 | Primer chiPh-as | herein |
| SEQ ID NO. 119 | Primer Gibson-PCS-s | herein |
| SEQ ID NO. 120 | Primer Gibson-PCS-as | herein |
| SEQ ID NO. 121 | Primer Gibson-PCS-short-s | herein |

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A coryneform bacteria cell with an increased provision of Malonyl-CoA compared to its archetype, wherein the regulation and/or expression of one or more of genes fasB, gltA, accBC and accD1, and/or the functionality of the enzymes encoded by each gene is modified in a targeted manner.

2. The coryneform bacteria cell according to claim 1, wherein the cell has one or more targeted modifications selected from the group comprising
a. Reduced or eliminated functionality of the fatty acid synthase FasB;
b. Mutation or partial or complete deletion of the fatty acid synthase encoding gene fasB;
c. Reduced functionality of the promoter operatively linked to the citrate synthase gene gtIA;
d. Reduced expression of the gene gltA coding for the citrate synthase CS;
e. Reduced or eliminated functionality of the operator binding sites (fasO) for the regulator FasR in the promoter regions of the genes accBC and accD1 coding for the acetyl-CoA carboxylase subunits;
f. Derepressed expression of the genes accBC and accD1 coding for the acetyl-CoA carboxylase subunits; and
g. One or more combinations of a)-f).

3. The coryneform bacteria cell according to claim 1, wherein the functionality of the fatty acid synthase FasB is reduced or turned off and/or the gene fasB coding for the fatty acid synthase is purposefully mutated, or is partially or completely deleted.

4. The coryneform bacteria cell according to claim 1, wherein the expression of the gene gltA coding for the citrate synthase is reduced by mutation of the operatively linked promoter.

5. The coryneform bacteria cell according to claim 1, wherein functionality of the operator binding sites (fasO) for the regulator FasR in the promoter regions of the genes accBC and accD1 coding for the acetyl-CoA carboxylase subunits is reduced or turned off and the expression of the genes accBC and accD1 coding for the acetyl-CoA carboxylase subunits is derepressed, preferably increased.

6. The coryneform bacteria cell according to claim 1, wherein the cell comprises a combination of reduced expression and/or activity of the citrate synthase (CS) and deregulated, increased expression and/or activity of the acetyl-CoA carboxylase subunits (AccBC and AccD1).

7. The coryneform bacteria cell according to claim 1, wherein the cell comprises a combination of reduced expression and/or activity of the citrate synthase (CS) and deregulated, increased expression and/or activity of the acetyl-CoA carboxylase subunits (AccBC and AccD1) and reduced or eliminated functionality of the fatty acid synthase FasB.

8. The coryneform bacteria cell according to claim 1, wherein the cell comprises a protein comprising a fatty acid synthase FasB isolated from coryneform bacteria whose functionality is reduced or turned off for the increased provision of malonyl-CoA in coryneform bacteria, wherein the amino acid sequence has at least 70% identity to the amino acid sequence selected from the group comprising SEQ ID NO. 2, 4, 6, 8 and 10 or fragments or alleles thereof.

9. The coryneform bacteria cell according to claim 1, wherein the cell comprises a nucleic acid sequence coding for a fatty acid synthase FasB from coryneform bacteria whose functionality is reduced or turned off, selected from the group comprising of:
a. a nucleic acid sequence containing at least 70% identity to the nucleic acid sequence selected from the group of SEQ ID NO. 1, 3, 5, 7 and 9 or fragments thereof,
b. a nucleic acid sequence which, under stringent conditions, hybridizes with a complementary sequence of a nucleic acid sequence selected from the group of SEQ ID NO. 1, 3, 5, 7 and 9 or fragments thereof,
c. a nucleic acid sequence selected from the group of SEQ ID NO. 1, 3, 5, 7 and 9 or fragments thereof, and
d. a nucleic acid sequence coding for a fatty acid synthase FasB corresponding to each of the nucleic acids according to a)-c) but which differs from these nucleic acid sequences according to a)-c) by the degeneracy of the genetic code or functionally neutral mutations,
for the increased provision of malonyl-CoA in coryneform bacteria.

10. (canceled)

11. The coryneform bacteria cell according to claim 1, wherein the cell has one or more targeted modifications selected from the group comprising of
a. Reduced or eliminated functionality of the fatty acid synthase FasB with at least 70% identity to the amino acid sequence selected from the group comprising SEQ ID NO. 2, 4, 6, 8 and 10 or fragments or alleles thereof,
b. Mutation or partial or complete deletion of the fatty acid synthase-encoding gene fasB with a nucleic acid sequence containing at least 70% identity to the nucleic acid sequence selected from the group of SEQ ID NO. 1, 3, 5, 7 and 9 or fragments thereof,
c. Reduced functionality of the promoter operatively linked to the citrate synthase gene gltA according to SEQ ID NO. 11;
d. Reduced expression of the gene gltA coding for the citrate synthase (CS);
e. Reduced or eliminated functionality of the operator binding sites (fasO) for the regulator FasR in the promoter regions of the genes accBC and accD1 coding for the acetyl-CoA carboxylase subunits according to SEQ ID NO. 13 and 15;
f. Derepressed expression of the genes accBC and accD1 coding for the acetyl-CoA carboxylase subunits; and
g. One or more combinations of a)-f).

12. The coryneform bacteria cell according to claim 1, wherein the modifications are chromosomally encoded.

13. The coryneform bacteria cell according to claim 1, wherein the cell is non-recombinantly altered (non-GVO).

14. The coryneform bacteria cell according to claim 1, wherein the cell is selected from the group comprising of Corynebacterium and Brevibacterium, preferably Corynebacterium glutamicum, particularly preferred Corynebacterium glutamicum ATCC 13032, Corynebacterium acetoglutamicum, Corynebacterium thermoaminogenes, Brevibacterium flavum, Brevibacterium lactofermentum and Brevibacterium divaricatum.

15. The coryneform bacteria cell according claim 1, wherein the cell comprises a catabolic pathway of aromatic components, wherein the pathway is turned off.

16. The coryneform bacteria cell according to claim 15, wherein the functionality and/or activity of the enzymes or the expression of the genes coding them involved in the catabolic pathway of aromatic components are turned off by deletions of the gene clusters cg0344-47 (phdBCDE operon), cg2625-40 (cat, ben and pca), cg 1226 (pobA) and cg0502 (qsuB).

17. The coryneform bacteria cell according to claim 1, wherein the cell comprises genes coding for a feedback-resistant 3-deoxy-D-arabinoheptulosonate-7-phosphate synthase (aroH), preferably from E. coli, and for a tyrosine ammonium lyase (tal), preferably from Flavobacterium johnsoniae.

18. The coryneform bacteria cell according to claim 1, wherein the cell additionally-further comprises enzymes derived from plants or the genes coding them for polyphenol or polyketide synthesis.

19. The coryneform bacteria cell according to claim 1, wherein the cell comprises a protein with an increased 5,7-dihydroxy-2-methylchromone synthase activity (PCSₛₕₒᵣₜ) for the synthesis of polyketides in coryneform bacteria, wherein the amino acid sequence has at least 70% identity to the amino acid sequence according to SEQ ID NO. 22 or fragments or alleles thereof.

20. The coryneform bacteria cell according to claim 1, wherein the cell comprises a nucleic acid sequence (pcsₛₕₒᵣₜ) coding for a 5,7-dihydroxy-2-methylchromone synthase with increased activity for polyketide production in coryneform bacteria selected from the group comprising of:
a. a nucleic acid sequence containing at least 70% identity to the nucleic acid sequence according to SEQ ID NO. 21 or fragments thereof,
b. a nucleic acid sequence which, under stringent conditions, hybridizes with a complementary sequence of a nucleic acid sequence according to SEQ ID NO. 21 or fragments thereof,
c. a nucleic acid sequence according to SEQ. ID NO. 21 or fragments thereof, or
d. a nucleic acid sequence coding for a 5,7-dihydroxy-2-methylchromone synthase (PCSₛₕₒᵣₜ) corresponding to each of the nucleic acids in accordance with a)-c) which is adapted to the codon usage of coryneform bacteria, and
e. that differs from these nucleic acid sequences in accordance with a)-d) by the degeneracy of the genetic code or by function-neutral mutations.

21. The coryneform bacteria cell according to claim 1, wherein the cell comprises one or more genes derived from plants for polyphenol or polyketide production selected from the group comprising of genes 4cl, sts, chs, chi and pcs.

22. The coryneform bacteria cell according to claim 21, wherein the plant genes are present under the expression control of an inducible promoter.

23. The coryneform bacteria cell according to claim 1, wherein the cell comprises gene 4clPc as chromosomal coding under the expression control of an inducible promoter.

24. (canceled)

25. The coryneform bacteria cell according to claim 1, wherein the cell comprises genes selected from the group comprising of
a. 4cl and sts for the synthesis of polyphenols,
b. chs and chi for the synthesis of polyphenols,
c. pcsₛₕₒᵣₜ for the synthesis of polyketides,
under the control of an inducible promoter.

26. The coryneform bacteria cell according to claim 1, wherein the cell comprises the genes selected from the group comprising of
a. fasB and/or gltA and/or accBC and accD1 or combinations thereof whose functionality and/or expression is specifically modified for an increased provision of malonyl-CoA, and
b. cg0344-47 (phdBCDE operon), cg2625-40 (cat, ben and pca), cg1226 (pobA) and cg0502 (qsuB), whose functionality for the degradation of aromatic components is switched off, and
c. pcsₛₕₒᵣₜ coding for a protein with an increased 5,7-dihydroxy-2-methylchromone synthase activity (PCSₛₕₒᵣₜ) for the synthesis of polyketides,
d. optionally aroFI and tal for the precursors synthesis of polyphenols starting from glucose,
e. 4cl and sts for the synthesis of polyphenols, and
f. chs and chi for the synthesis of polyphenols.

27. (canceled)

28. A method for the increased provision of malonyl-CoA in coryneform bacteria comprising the steps of:
a. providing a solution comprising water and a C6 carbon source; and
b. converting the C6 carbon source in a solution according to step a) to malonyl-CoA in the presence of a coryneform bacteria cell according to a claim 1.

29. A method for the microbial production of polyphenols or polyketides in coryneform bacteria, comprising the steps of:
a. providing a solution containing water and a C6 carbon source,
b. converting the C6 carbon source in a solution according to step a) to polyphenols or polyketides in the presence of a coryneform bacteria cell according to claim 1, wherein malonyl-CoA is first provided at an elevated concentration as an intermediate and further reacted for microbial synthesis of polyphenols or polyketides; and
c. inducing expression of plant genes under the control of an inducible promoter by addition of a suitable inducer in step b).

30. The method for polyphenol production according to claim 29, wherein the solution in step b) is supplemented with a polyphenol precursor.

31. The method according to claim 29, wherein cultivation takes place in a discontinuous or continuous mode.

32-35. (canceled)


SEQUENCE LISTING (121 sequences)

SEQ ID NO: 1 - DNA, 8991 bp, Corynebacterium glutamicum
gtgaccgaattgagcaggaacttcggggccagccgactgattaaccgctttggccaggagccttttgccttcgctttcgccggccaaggatatgactggttgaagacccttcgtgccgcggttgccgcaggtgcaggcaccaatgttagtgacatcgtcgagcgcgcaaatgcgctgcttgcactagttgcagatgatctcattggcacccttccatttggtttcgatccagtggcttgggctaacaactccgaagatccagctttcgatactgcacaatctgcagtgagcgtgccgggtatctttgtctcccagatcgcaaccctggattcccttgaggcgcagcgccttgatgtggatcaggctgtgtccagcattggtcattcccagggcgtattgggcgtgcacctgctcaatgatgcgactcgtgctgatgaactcgttgccattgcgcagttgatcggtgcagcgatcacccgcaccgcacgcatgacgggcctgatcgcgcagggcgacaacatgccgatgctgtcgatcgccggaatttcccgcgaacagcttcagcaagctatcgacgcggcctgcgccgaagtccctgcggagatccgcccggttatcggtctgcgcaactcacgcgattcttatgttttggttggccgcccagacgacaacgctcgcgttgttaaggtcattgaggcaatggctgccaaggataagaaggccattgaagataagctgcgcggcggttccgcgttcagcccccgtattactccgctgaaggtgcaggctgctttccatcacccagctatgaacatggctgtggagcagaccgtggcgtgggcaaccactgctggtttggatgtggaactcacccgcgagatcgccgctgatgttttggttaaccctgtcgattgggtagcacgcgtcaacgaagcgtatgaggctggcgctcgctggttcctcgacgttggaccagatggtggcatcgttaagctgactgccaacatccttgagggccgcggcgcggattccttctatgttggtgacgccgcaggccaggccaagatatttgatgctggcatggcacctgaacttccagtggattaccaggagttcgcaccacgcgttgagcacgttgatggaaccccacgcctggttaccaagttcactgagctgaccggccgcaccccaatgatgctggctggcatgaccccaaccaccgttgaccctgccattgttgcagccgctgcaaacggtggacactgggctgagctcgctggtggcggacaggttaccccagagctgctggaaacccacatcgcacagctcaccgacatgcttgagccaggtatcaacgcccagttcaactccatgttcttggatccatacctgtggaagatgcagattggtggcaagcgccttgttcctaaggcccgcgctaatggtgcatccatcgacggcatcgtcatcaccgccggcattcctgaaaaggatgaagctgttgcattggtcaaggaactgatgcgtgatggtttcccttggatcgcattcaagccaggtgccatcaagcaggttaactctgtgttggctatcgctaaggaagttccagaactccccatcatcattcagattgagggtggcgttgcaggtggacaccactcttgggaagacctcgatgagctgctgatcgccacctacggcaaggtccgcgcactggataacgtggtgctgtgtgtcggcggtggcattggctcacctgagcgcgctgctgattacgtcaccggttcctggtccacttcctacggcctgccagctatgcctgttgatggcatcttggtgggtaccgctgcgatggcaaccaagaaagcaaccacctcccaggccgtcaaggaacttcttgtttccacccagggctctgatgaatgggttcctgctggtggcgcaaagaacggaatggcatctggccgttcccagcttggcgcagacatccacgagatcgacaactcctttgctaaggctggacgccttcttgatgaggttgcaggcgatgagacggctgtgcaggcgcgccgggatgagatcattgaagcgattggcaagaccgccaaggtgtacttcggtgacatcggatccatgacttacgagcagtggctcaaccgctacctcgagctgtctggccctgttgatggtcagtggattgatgcttcctgggctgcacgttttgcccagatgctggagcgtgccgaggcgcgtttgatcgagcaggatcatggccaatttgagccaagcctgacggtggaggatggcgtcgacaagcttgttgctgcttacccgcatgccgcaaccgacctgctcaccccggctgatgtcgcctggttcttgggcctgtgccgcacgccgggcaagcctgtgaactttgtgcccgtcattgataaggacgtgcgtcgctggtggcgctcggactccctgtggcagtcccacgatgatcgctacaccgctgatcaggtggctattatccctggtgtcgtcgccgttgctggcatcaccaaggccaacgaacctgtcgctgacctgcttgatcgctttgtcgacgccaccatcgagcgcatcgatgagcacgattcccgctcccgcgacatcatgggcaaagtgctttcctcacctggcacattctgggctggccgcaacatcccatcggtgatccacagccttgggcatgctgacaagtggtcccgctccgaattcgaagcattccatagcccaaccggcgccaacttggtgtacgaagacgccgagcacgcgatgctgactgtgcctttggcgggttccaccgcattcggcaccaccgctgagctgaaaatccgtttcaccagccccatcgacgctctgccaagcgctgtcccactggtcacccaggaagacgctgaagccgcgatgggtgaactgacccgcatcgcagctggcggcaccctggcaactgtgaacaatggcaccgctacctgggaaacctccgtcgatgccggcgtcatcgctgactacaacaacgtcaccgcaggctacctgccagcatccgttgttcctgcacacaccgcacctgacgtgctggttggccgcgcatggccagcagttttcgctgccgtaaagtccgcagtcatcccaggcaccgattccgcatccgttgtggaaggcatgctgtccctggttcacctggagcaccacattgtgctcaagtccgatgtcccaaccgacggcgcgctgaaggtttccgcgactgccgatgaggtagtcgataccgacctgggtcgcctcgtgatcgtgcgcgcagaaatcgccgacgcagaaggcaacctgattgctacgttggctgagcgtttcgcgatccgcggacgcaagggcaacgctgtcgcacgcaccaacacctccgcactgccaaccaccgtggacaccccacgctcagctcgcgcagtggcaaccgttgttgcacctgaatccatgcgcccattcgctgtgatctccggtgaccgcaacccaattcacgtctctgatgttgcggcttccctggctggtctgccaggtgtgatcgtgcacggcatgtggacctctgccatcggtgaactgatcgccggtgcagcattcaacgatgagcagatccaaactcccgcagccaaggtcgtggaatacaccgcaaccatgctggcaccagttcttccaggtgaagaaattgagttcagcgttgagcgctccgcagtggacaaccgcccaggaatgggagaggtccgcaccgttaccgcaaccgtcaacggcaacttagtgcttaccgccaccgctgttgtggcagctccatctactttctacgcattcccaggccagggcattcagtcccagggcatgggtatggaagcacgccgtaactctcaggcagctcgcgctatctgggaccgcgccgatgcacacacccgcaataagctgggcttctccatcgtggaaatcgtggaaaacaacccacgcgaagtaaccgtggcaggggagaagttcttccacccagacggcgttttgtacctcacccagttcacccaggtgggcatggcaactctgggcgttgctcagatcgctgaaatgcgtgaagcacatgccttgaaccagcgtgcatactttgctggacactccgttggtgagtacaacgcgcttgctgcatatgctggtgtgctgtccctggaatccgttctggagatcgtttaccgtcgtggcttgaccatgcaccgcttggtggatcgcgatgaaaacggtctgtccaactacgcgctcgcagctcttcgccccaacaagatgggtctgaccgcagacaacgttttcgattacgttgcgtctgtttccgaagcttccggtgaattcctggagatcgttaactacaacttggctggcctgcagtacgcagttgctggaacccaggctggtcttgccgcccttcgtgccgatgttgagaaccgtgcaccaggtcagcgtgccttcattttgatccctggcattgacgtgccattccactcctccaagctgcgcgacggtgtgggcgcgttccgtgagcaccttgattccctgatcccagctgagctggatctggatgtgctggttggccgctacattccaaacttggtggctcgcccattcgaactcactgaagagttcgtggcatccatggcagaagtggtggagtccacctatgtcaatgagatcttggctgatttcaaggctgcttccgccgataagcagaagcttgcccgcacgttgcttattgagctgcttgcatggcagttcgcatcacctgtgcgctggatcgagactcaggatctgttgatcaagggccttcaagctgagcgtttcgtggaggtcggtgttggctctgctccaacgcttgccaacatgatgggccagaccctgcgccttcctcagtacgcggacgccaccattgaggtgttaaacattgagcgcgatcgcccagttgtgttcgctaccgatgaggttgtgcgtgaagtggcggttgaagagaccccagcagctcctgcagaaaccactgaaaccccagcaaccccagcaaccccagcccctgttgcagctgcagcccctgccaccggcggccctcgcccagatgacatcagcttcactccttctgatgccactgaaatgctcatcgctatctggaccaaggttcgcccagatcagatgggtgccactgattccatcgagaccctggttgagggcgtgtcctctcgccgtaaccagctcctgctggatcttggtgtggagttcggcctcggcgcaattgacggagcagccgatgctgagctcggtgatctaaaggtcaccgtgtccaagatggctaagggctacaaggcgtttggccctgtgctctccgatgctgcagctgatgccctgcgtcgcctcactggtcctaccggtaagcgcccgggatacatcgcagagcgcgtcaccggcacgtgggaattgggccagggctgggctgaccacgtggtcgctgaagttgtgatcggcgcccgcgaaggcgcatccctgcgcggcggcgacctggcgtcactgtctcctgcaagcccagcgtctgcatcagatcttgattcgcttatcgacgcagccgtccaggccgtagcctcccgccgcggcgttgcggtctccctgccttcagcaggcggcgctgccggtggcgtggttgattccgcagctcttggcgagtttgcagagcaggtcaccggacacgatggtgtgcttgctcaggcagcccgcaccatcttgacccagttgggtcttgataagccagcaaccgtttccgtggaagacaccgcagaggaagacctctacgagttggtctccaaggaactcggttctgattggccacgtcaggttgcaccaagcttcgatgaagaaaaggttgttctgcttgatgaccgttgggcttctgcgcgtgaggatctctcccgcgttgctcttggcgaactcgcagcaactgatatcgatgtcacaggcgcaggcgaagctgttgcagcacaggctgaattctttggacttgatgatctcgcagctcaggctcgcgaccaaagctccttggactacgccgacgatgttgcggtcgtaaccggcggatcgccgaactcgatcgcggcggcagtcgtcgaaaagcttcttgctggcggtgcaactgtcattgctacgacctccaacctcggccatgaccgcctggagttctacaaggatctctacgcacgttccgcacgcggcacggcagcactgtggatcgtggcggctaacttgagctcctactcagacatcgacgccatcatcaactgggtcggatccgagcagaccaccaccgtcaacggcgcatccaagctggtcaagccagctttggtccctaccttgctgttcccattcgcggcacctcgcgtgtccggatccatggcagatgcaggcccacaggcagaatcccagatgcgacttctgctctggtctgttgagcgcctcatcgcaggtcttgcgccattgggctcctccatcaacgtgggtcaccgcctgcacgtggtcatcccaggttcaccaaaccgtggacgcttcggtggcgatggtgcatacggtgaatccaaggcagctctcgacgccgtggttacccgttggaacgcagagcaagctgcatggggagcacacacctccctcgtgcacgctcacatcggttgggttcgcggcaccggcctcatgggcggcaacgatcctttggtcaaggcagctgaagaagcaggcgtggaaacctactccacccaagaaattgcagagaaactgctgtcccaggcaacttccactgttcgcgagcaggcagcatccgcgccaatcaccgtcgacttcactggcggacttggtgaatctgatctgaacctggcggaaatggcacgtgcagaagcagctaaggcagctaacgcaccagtggttgaggctccacgcacagtggcagcactgccaactccttaccgaccagtggttcaaaccacccctgatttcgcaggtcaagtcacccaaaaccttgacgagatggtcgtcatcgttggcgccggcgagctcggcccactgggttctgcacgtacgcgtttcgacgccgaactcaacggttccctctccgccgcgggtgtcatcgaacttgcatggacgatgggacttatccactgggatgaagatccaaagccaggctggtacgacgactccgacgacgcagtggccgaagaagacatcttcgaccgctaccacgacgaagtcatggcacgcgttggtgtccgcaagtacaatgacatgcctgagtacggcatgatcgacaactttgcaccagagctgaccaccgtctacctcgaccaggacctcaccttcaacgtgggatcccgcgaagaggcactgacctacgtcgactccgagccagaactcacctttgcttctttcgacgaagcagcaggggagtggaaggtcactcgcaaggcaggctccgcaatccgcgtacctcgccgcatggcgatgacccgcttcgttggtggacaggttcctaaggacttcgacccagctgtgtggggcattccagctgacatggtggacaacctggacaccgtcgcgctgtggaacattgtctgtactgtcgacgccttcctgtccgctggattcaccccagcagagctgcttgcttccgttcacccagcacgcgtgtcctctacccaaggcaccggcatgggcggcatggaatccctccgtggcatctacgtcgaccgcattctggcagagccacgcgccaacgacgttctgcaggaagcactgcccaacgttgttgcagctcacgtcatgcagtcctacgtcggtggctacggacagatgatccacccagtcgcagcttgtgcaaccgcagctgtttctgtggaagaagcactggacaagatccgcatcggcaagtccgacttcgttgtcgcaggtggcttcgatgccctgtccgttgaaggcatcaccggcttcggcgacatggcagcaaccgccgactccgcagagatggaaggcaagggaattgagcaccgcttcttctcccgcgccaacgaccgccgccgcggtggattcatcgaatccgaaggtggcggaaccgtccttctggcacgcggatcactcgcagctgacctgggccttccagtactcggtgtcatcggattcgcagagtcctttgcagatggtgcccacacctccatcccagccccaggcctcggtgcccttggtgctgctcgcgatggtgtggaatctcgccttgcagtagcactgcgttccgtcggtgtctctgctgatgagatctccattatctccaagcacgacacctccaccaacgcgaatgatccaaacgagtccgacctgcacgagcgcatcgcatccgctatcggtcgtgcagacggcaacccgatgtacgtgatttcccagaagtcactcaccggacacgccaagggtggtgcagcagcattccagatgatcggtctcacccaggtcctccgatccggactggtgccagccaaccgcgcactcgactgcgttgacccagtactgtccaagcattcccacctcgtctggctgcgcaagccactagaccttcgtgcgaaggcaccaaaggcaggtcttgttacctcccttggcttcggacacgtctccgctctggttgcgattgttcacccagacgccttctatgaggcagttcgtgtggcacgtggtgctgaggcagctgacgtatggcgcgcatccgcgatcgctcgcgaagaagcaggccttcgtaccatcgtcgccggtatgcacggtggcgtactgtacgaacgcccagtcgagcgcaacctcggtgtccacggagacgcagctaaggaagttgaagctgcagtcctcctggattcccgcgcccgcctagttgacggtgtcctccgcgccgaaggctag

SEQ ID NO: 2 - protein, 2996 aa, Corynebacterium glutamicum
MTELSRNFGASRLINRFGQEPFAFAFAGQGYDWLKTLRAAVAAGAGTNVSDIVERANALLALVADDLIGTLPFGFDPVAWANNSEDPAFDTAQSAVSVPGIFVSQIATLDSLEAQRLDVDQAVSSIGHSQGVLGVHLLNDATRADELVAIAQLIGAAITRTARMTGLIAQGDNMPMLSIAGISREQLQQAIDAACAEVPAEIRPVIGLRNSRDSYVLVGRPDDNARVVKVIEAMAAKDKKAIEDKLRGGSAFSPRITPLKVQAAFHHPAMNMAVEQTVAWATTAGLDVELTREIAADVLVNPVDWVARVNEAYEAGARWFLDVGPDGGIVKLTANILEGRGADSFYVGDAAGQAKIFDAGMAPELPVDYQEFAPRVEHVDGTPRLVTKFTELTGRTPMMLAGMTPTTVDPAIVAAAANGGHWAELAGGGQVTPELLETHIAQLTDMLEPGINAQFNSMFLDPYLWKMQIGGKRLVPKARANGASIDGIVITAGIPEKDEAVALVKELMRDGFPWIAFKPGAIKQVNSVLAIAKEVPELPIIIQIEGGVAGGHHSWEDLDELLIATYGKVRALDNVVLCVGGGIGSPERAADYVTGSWSTSYGLPAMPVDGILVGTAAMATKKATTSQAVKELLVSTQGSDEWVPAGGAKNGMASGRSQLGADIHEIDNSFAKAGRLLDEVAGDETAVQARRDEIIEAIGKTAKVYFGDIGSMTYEQWLNRYLELSGPVDGQWIDASWAARFAQMLERAEARLIEQDHGQFEPSLTVEDGVDKLVAAYPHAATDLLTPADVAWFLGLCRTPGKPVNFVPVIDKDVRRWWRSDSLWQSHDDRYTADQVAIIPGVVAVAGITKANEPVADLLDRFVDATIERIDEHDSRSRDIMGKVLSSPGTFWAGRNIPSVIHSLGHADKWSRSEFEAFHSPTGANLVYEDAEHAMLTVPLAGSTAFGTTAELKIRFTSPIDALPSAVPLVTQEDAEAAMGELTRIAAGGTLATVNNGTATWETSVDAGVIADYNNVTAGYLPASVVPAHTAPDVLVGRAWPAVFAAVKSAVIPGTDSASVVEGMLSLVHLEHHIVLKSDVPTDGALKVSATADEVVDTDLGRLVIVRAEIADAEGNLIATLAERFAIRGRKGNAVARTNTSALPTTVDTPRSARAVATVVAPESMRPFAVISGDRNPIHVSDVAASLAGLPGVIVHGMWTSAIGELIAGAAFNDEQIQTPAAKVVEYTATMLAPVLPGEEIEFSVERSAVDNRPGMGEVRTVTATVNGNLVLTATAVVAAPSTFYAFPGQGIQSQGMGMEARRNSQAARAIWDRADAHTRNKLGFSIVEIVENNPREVTVAGEKFFHPDGVLYLTQFTQVGMATLGVAQIAEMREAHALNQRAYFAGHSVGEYNALAAYAGVLSLESVLEIVYRRGLTMHRLVDRDENGLSNYALAALRPNKMGLTADNVFDYVASVSEASGEFLEIVNYNLAGLQYAVAGTQAGLAALRADVENRAPGQRAFILIPGIDVPFHSSKLRDGVGAFREHLDSLIPAELDLDVLVGRYIPNLVARPFELTEEFVASMAEVVESTYVNEILADFKAASADKQKLARTLLIELLAWQFASPVRWIETQDLLIKGLQAERFVEVGVGSAPTLANMMGQTLRLPQYADATIEVLNIERDRPVVFATDEVVREVAVEETPAAPAETTETPATPATPAPVAAAAPATGGPRPDDISFTPSDATEMLIAIWTKVRPDQMGATDSIETLVEGVSSRRNQLLLDLGVEFGLGAIDGAADAELGDLKVTVSKMAKGYKAFGPVLSDAAADALRRLTGPTGKRPGYIAERVTGTWELGQGWADHVVAEVVIGAREGASLRGGDLASLSPASPASASDLDSLIDAAVQAVASRRGVAVSLPSAGGAAGGVVDSAALGEFAEQVTGHDGVLAQAARTILTQLGLDKPATVSVEDTAEEDLYELVSKELGSDWPRQVAPSFDEEKVVLLDDRWASAREDLSRVALGELAATDIDVTGAGEAVAAQAEFFGLDDLAAQARDQSSLDYADDVAVVTGGSPNSIAAAVVEKLLAGGATVIATTSNLGHDRLEFYKDLYARSARGTAALWIVAANLSSYSDIDAIINWVGSEQTTTVNGASKLVKPALVPTLLFPFAAPRVSGSMADAGPQAESQMRLLLWSVERLIAGLAPLGSSINVGHRLHVVIPGSPNRGRFGGDGAYGESKAALDAVVTRWNAEQAAWGAHTSLVHAHIGWVRGTGLMGGNDPLVKAAEEAGVETYSTQEIAEKLLSQATSTVREQAASAPITVDFTGGLGESDLNLAEMARAEAAKAANAPVVEAPRTVAALPTPYRPVVQTTPDFAGQVTQNLDEMVVIVGAGELGPLGSARTRFDAELNGSLSAAGVIELAWTMGLIHWDEDPKPGWYDDSDDAVAEEDIFDRYHDEVMARVGVRKYNDMPEYGMIDNFAPELTTVYLDQDLTFNVGSREEALTYVDSEPELTFASFDEAAGEWKVTRKAGSAIRVPRRMAMTRFVGGQVPKDFDPAVWGIPADMVDNLDTVALWNIVCTVDAFLSAGFTPAELLASVHPARVSSTQGTGMGGMESLRGIYVDRILAEPRANDVLQEALPNVVAAHVMQSYVGGYGQMIHPVAACATAAVSVEEALDKIRIGKSDFVVAGGFDALSVEGITGFGDMAATADSAEMEGKGIEHRFFSRANDRRRGGFIESEGGGTVLLARGSLAADLGLPVLGVIGFAESFADGAHTSIPAPGLGALGAARDGVESRLAVALRSVGVSADEISIISKHDTSTNANDPNESDLHERIASAIGRADGNPMYVISQKSLTGHAKGGAAAFQMIGLTQVLRSGLVPANRALDCVDPVLSKHSHLVWLRKPLDLRAKAPKAGLVTSLGFGHVSALVAIVHPDAFYEAVRVARGAEAADVWRASAIAREEAGLRTIVAGMHGGVLYERPVERNLGVHGDAAKEVEAAVLLDSRARLVDGVLRAEG

SEQ ID NO: 3 - DNA, 8991 bp, Corynebacterium glutamicum
gtgaccgaattgagcaggaacttcggggccagccgactgattaaccgctttggccaggagccttttgccttcgctttcgccggccaaggatatgactggttgaagacccttcgtgccgcggttgccgcaggtgcaggcaccaatgttagtgacatcgtcgagcgcgcaaatgcgctgcttgcactagttgcagatgatctcattggcacccttccatttggtttcgatccagtggcttgggctaacaactccgaagatccagctttcgatactgcacaatctgcagtgagcgtgccgggtatctttgtctcccagatcgcaaccctggattcccttgaggcgcagcgccttgatgtggatcaggctgtgtccagcattggtcattcccagggcgtattgggcgtgcacctgctcaatgatgcgactcgtgctgatgaactcgttgccattgcgcagttgatcggtgcagcgatcacccgcaccgcacgcatgacgggcctgatcgcgcagggcgacaacatgccgatgctgtcgatcgccggaatttcccgcgaacagcttcagcaagctatcgacgcggcctgcgccgaagtccctgcggagatccgcccggttatcggtctgcgcaactcacgcgattcttatgttttggttggccgcccagacgacaacgctcgcgttgttaaggtcattgaggcaatggctgccaaggataagaaggccattgaagataagctgcgcggcggttccgcgttcagcccccgtattactccgctgaaggtgcaggctgctttccatcacccagctatgaacatggctgtggagcagaccgtggcgtgggcaaccactgctggtttggatgtggaactcacccgcgagatcgccgctgatgttttggttaaccctgtcgattgggtagcacgcgtcaacgaagcgtatgaggctggcgctcgctggttcctcgacgttggaccagatggtggcatcgttaagctgactgccaacatccttgagggccgcggcgcggattccttctatgttggtgacgccgcaggccaggccaagatatttgatgctggcatggcacctgaacttccagtggattaccaggagttcgcaccacgcgttgagcacgttgatggaaccccacgcctggttaccaagttcactgagctgaccggccgcaccccaatgatgctggctggcatgaccccaaccaccgttgaccctgccattgttgcagccgctgcaaacggtggacactgggctgagctcgctggtggcggacaggttaccccagagctgctggaaacccacatcgcacagctcaccgacatgcttgagccaggtatcaacgcccagttcaactccatgttcttggatccatacctgtggaagatgcagattggtggcaagcgccttgttcctaaggcccgcgctaatggtgcatccatcgacggcatcgtcatcaccgccggcattcctgaaaaggatgaagctgttgcattggtcaaggaactgatgcgtgatggtttcccttggatcgcattcaagccaggtgccatcaagcaggttaactctgtgttggctatcgctaaggaagttccagaactccccatcatcattcagattgagggtggcgttgcaggtggacaccactcttgggaagacctcgatgagctgctgatcgccacctacggcaaggtccgcgcactggataacgtggtgctgtgtgtcggcggtggcattggctcacctgagcgcgctgctgattacgtcaccggttcctggtccacttcctacggcctgccagctatgcctgttgatggcatcttggtgggtaccgctgcgatggcaaccaaggaagcaaccacctcccaggccgtcaaggaacttcttgtttccacccagggctctgatgaatgggttcctgctggtggcgcaaagaacggaatggcatctggccgttcccagcttggcgcagacatccacgagatcgacaactcctttgctaaggctggacgccttcttgatgaggttgcaggcgatgagacggctgtgcaggcgcgccgggatgagatcattgaagcgattggcaagaccgccaaggtgtacttcggtgacatcggatccatgacttacgagcagtggctcaaccgctacctcgagctgtctggccctgttgatggtcagtggattgatgcttcctgggctgcacgttttgcccagatgctggagcgtgccgaggcgcgtttgatcgagcaggatcatggccaatttgagccaagcctgacggtggaggatggcgtcgacaagcttgttgctgcttacccgcatgccgcaaccgacctgctcaccccggctgatgtcgcctggttcttgggcctgtgccgcacgccgggcaagcctgtgaactttgtgcccgtcattgataaggacgtgcgtcgctggtggcgctcggactccctgtggcagtcccacgatgatcgctacaccgctgatcaggtggctattatccctggtgtcgtcgccgttgctggcatcaccaaggccaacgaacctgtcgctgacctgcttgatcgctttgtcgacgccaccatcgagcgcatcgatgagcacgattcccgctcccgcgacatcatgggcaaagtgctttcctcacctggcacattctgggctggccgcaacatcccatcggtgatccacagccttgggcatgctgacaagtggtcccgctccgaattcgaagcattccatagcccaaccggcgccaacttggtgtacgaagacgccgagcacgcgatgctgactgtgcctttggcgggttccaccgcattcggcaccaccgctgagctgaaaatccgtttcaccagccccatcgacgctctgccaagcgctgtcccactggtcacccaggaagacgctgaagccgcgatgggtgaactgacccgcatcgcagctggcggcaccctggcaactgtgaacaatggcaccgctacctgggaaacctccgtcgatgccggcgtcatcgctgactacaacaacgtcaccgcaggctacctgccagcatccgttgttcctgcacacaccgcacctgacgtgctggttggccgcgcatggccagcagttttcgctgccgtaaagtccgcagtcatcccaggcaccgattccgcatccgttgtggaaggcatgctgtccctggttcacctggagcaccacattgtgctcaagtccgatgtcccaaccgacggcgcgctgaaggtttccgcgactgccgatgaggtagtcgataccgacctgggtcgcctcgtgatcgtgcgcgcagaaatcgccgacgcagaaggcaacctgattgctacgttggctgagcgtttcgcgatccgcggacgcaagggcaacgctgtcgcacgcaccaacacctccgcactgccaaccaccgtggacaccccacgctcagctcgcgcagtggcaaccgttgttgcacctgaatccatgcgcccattcgctgtgatctccggtgaccgcaacccaattcacgtctctgatgttgcggcttccctggctggtctgccaggtgtgatcgtgcacggcatgtggacctctgccatcggtgaactgatcgccggtgcagcattcaacgatgagcagatccaaactcccgcagccaaggtcgtggaatacaccgcaaccatgctggcaccagttcttccaggtgaagaaattgagttcagcgttgagcgctccgcagtggacaaccgcccaggaatgggagaggtccgcaccgttaccgcaaccgtcaacggcaacttagtgcttaccgccaccgctgttgtggcagctccatctactttctacgcattcccaggccagggcattcagtcccagggcatgggtatggaagcacgccgtaactctcaggcagctcgcgctatctgggaccgcgccgatgcacacacccgcaataagctgggcttctccatcgtggaaatcgtggaaaacaacccacgcgaagtaaccgtggcaggggagaagttcttccacccagacggcgttttgtacctcacccagttcacccaggtggacatggcaactctgggcgttgctcagatcgctgaaatgcgtgaagcacatgccttgaaccagcgtgcatactttgctggacactccgttggtgagtacaacgcgcttgctgcatatgctggtgtgctgtccctggaatccgttctggagatcgtttaccgtcgtggcttgaccatgcaccgcttggtggatcgcgatgaaaacggtctgtccaactacgcgctcgcagctcttcgccccaacaagatgggtctgaccgcagacaacgttttcgattacgttgcgtctgtttccgaagcttccggtgaattcctggagatcgttaactacaacttggctggcctgcagtacgcagttgctggaacccaggctggtcttgccgcccttcgtgccgatgttgagaaccgtgcaccaggtcagcgtgccttcattttgatccctggcattgacgtgccattccactcctccaagctgcgcgacggtgtgggcgcgttccgtgagcaccttgattccctgatcccagctgagctggatctggatgtgctggttggccgctacattccaaacttggtggctcgcccattcgaactcactgaagagttcgtggcatccatggcagaagtggtggagtccacctatgtcaatgagatcttggctgatttcaaggctgcttccgccgataagcagaagcttgcccgcacgttgcttattgagctgcttgcatggcagttcgcatcacctgtgcgctggatcgagactcaggatctgttgatcaagggccttcaagctgagcgtttcgtggaggtcggtgttggctctgctccaacgcttgccaacatgatgggccagaccctgcgccttcctcagtacgcggacgccaccattgaggtgttaaacattgagcgcgatcgcccagttgtgttcgctaccgatgaggttgtgcgtgaagtggcggttgaagagaccccagcagctcctgcagaaaccactgaaaccccagcaaccccagcaaccccagcccctgttgcagctgcagcccctgccaccggcggccctcgcccagatgacatcagcttcactccttctgatgccactgaaatgctcatcgctatctggaccaaggttcgcccagatcagatgggtgccactgattccatcgagaccctggttgagggcgtgtcctctcgccgtaaccagctcctgctggatcttggtgtggagttcggcctcggcgcaattgacggagcagccgatgctgagctcggtgatctaaaggtcaccgtgtccaagatggctaagggctacaaggcgtttggccctgtgctctccgatgctgcagctgatgccctgcgtcgcctcactggtcctaccggtaagcgcccgggatacatcgcagagcgcgtcaccggcacgtgggaattgggccagggctgggctgaccacgtggtcgctgaagttgtgatcggcgcccgcgaaggcgcatccctgcgcggcggcgacctggcgtcactgtctcctgcaagcccagcgtctgcatcagatcttgattcgcttatcgacgcagccgtccaggccgtagcctcccgccgcggcgttgcggtctccctgccttcagcaggcggcgctgccggtggcgtggttgattccgcagctcttggcgagtttgcagagcaggtcaccggacacgatggtgtgcttgctcaggcagcccgcaccatcttgacccagttgggtcttgataagccagcaaccgtttccgtggaagacaccgcagaggaagacctctacgagttggtctccaaggaactcggttctgattggccacgtcaggttgcaccaagcttcgatgaagaaaaggttgttctgcttgatgaccgttgggcttctgcgcgtgaggatctctcccgcgttgctcttggcgaactcgcagcaactgatatcgatgtcacaggcgcaggcgaagctgttgcagcacaggctgaattctttggacttgatgatctcgcagctcaggctcgcgaccaaagctccttggactacgccgacgatgttgcggtcgtaaccggcggatcgccgaactcgatcgcggcggcagtcgtcgaaaagcttcttgctggcggtgcaactgtcattgctacgacctccaacctcggccatgaccgcctggagttctacaaggatctctacgcacgttccgcacgcggcacggcagcactgtggatcgtggcggctaacttgagctcctactcagacatcgacgccatcatcaactgggtcggatccgagcagaccaccaccgtcaacggcgcatccaagctggtcaagccagctttggtccctaccttgctgttcccattcgcggcacctcgcgtgtccggatccatggcagatgcaggcccacaggcagaatcccagatgcgacttctgctctggtctgttgagcgcctcatcgcaggtcttgcgccattgggctcctccatcaacgtgggtcaccgcctgcacgtggtcatcccaggttcaccaaaccgtggacgcttcggtggcgatggtgcatacggtgaatccaaggcagctctcgacgccgtggttacccgttggaacgcagagcaagctgcatggggagcacacacctccctcgtgcacgctcacatcggttgggttcgcggcaccggcctcatgggcggcaacgatcctttggtcaaggcagctgaagaagcaggcgtggaaacctactccacccaagaaattgcagagaaactgctgtcccaggcaacttccactgttcgcgagcaggcagcatccgcgccaatcaccgtcgacttcactggcggacttggtgaatctgatctgaacctggcggaaatggcacgtgcagaagcagctaaggcagctaacgcaccagtggttgaggctccacgcacagtggcagcactgccaactccttaccgaccagtggttcaaaccacccctgatttcgcaggtcaagtcacccaaaaccttgacgagatggtcgtcatcgttggcgccggcgagctcggcccactgggttctgcacgtacgcgtttcgacgccgaactcaacggttccctctccgccgcgggtgtcatcgaacttgcatggacgatgggacttatccactgggatgaagatccaaagccaggctggtacgacgactccgacgacgcagtggccgaagaagacatcttcgaccgctaccacgacgaagtcatggcacgcgttggtgtccgcaagtacaatgacatgcctgagtacggcatgatcgacaactttgcaccagagctgaccaccgtctacctcgaccaggacctcaccttcaacgtgggatcccgcgaagaggcactgacctacgtcgactccgagccagaactcacctttgcttctttcgacgaagcagcaggggagtggaaggtcactcgcaaggcaggctccgcaatccgcgtacctcgccgcatggcgatgacccgcttcgttggtggacaggttcctaaggacttcgacccagctgtgtggggcattccagctgacatggtggacaacctggacaccgtcgcgctgtggaacattgtctgtactgtcgacgccttcctgtccgctggattcaccccagcagagctgcttgcttccgttcacccagcacgcgtgtcctctacccaaggcaccggcatgggcggcatggaatccctccgtggcatctacgtcgaccgcattctggcagagccacgcgccaacgacgttctgcaggaagcactgcccaacgttgttgcagctcacgtcatgcagtcctacgtcggtggctacggacagatgatccacccagtcgcagcttgtgcaaccgcagctgtttctgtggaagaagcactggacaagatccgcatcggcaagtccgacttcgttgtcgcaggtggcttcgatgccctgtccgttgaaggcatcaccggcttcggcgacatggcagcaaccgccgactccgcagagatggaaggcaagggaattgagcaccgcttcttctcccgcgccaacgaccgccgccgcggtggattcatcgaatccgaaggtggcggaaccgtccttctggcacgcggatcactcgcagctgacctgggccttccagtactcggtgtcatcggattcgcagagtcctttgcagatggtgcccacacctccatcccagccccaggcctcggtgcccttggtgctgctcgcgatggtgtggaatctcgccttgcagtagcactgcgttccgtcggtgtctctgctgatgagatctccattatctccaagcacgacacctccaccaacgcgaatgatccaaacgagtccgacctgcacgagcgcatcgcatccgctatcggtcgtgcagacggcaacccgatgtacgtgatttcccagaagtcactcaccggacacgccaagggtggtgcagcagcattccagatgatcggtctcacccaggtcctccgatccggactggtgccagccaaccgcgcactcgactgcgttgacccagtactgtccaagcattcccacctcgtctggctgcgcaagccactagaccttcgtgcgaaggcaccaaaggcaggtcttgttacctcccttggcttcggacacgtctccgctctggttgcgattgttcacccagacgccttctatgaggcagttcgtgtggcacgtggtgctgaggcagctgacgtatggcgcgcatccgcgatcgctcgcgaagaagcaggccttcgtaccatcgtcgccggtatgcacggtggcgtactgtacgaacgcccagtcgagcgcaacctcggtgtccacggagacgcagctaaggaagttgaagctgcagtcctcctggattcccgcgcccgcctagttgacggtgtcctccgcgccgaaggctag

SEQ ID NO: 4 - protein, 2996 aa, Corynebacterium glutamicum
MTELSRNFGASRLINRFGQEPFAFAFAGQGYDWLKTLRAAVAAGAGTNVSDIVERANALLALVADDLIGTLPFGFDPVAWANNSEDPAFDTAQSAVSVPGIFVSQIATLDSLEAQRLDVDQAVSSIGHSQGVLGVHLLNDATRADELVAIAQLIGAAITRTARMTGLIAQGDNMPMLSIAGISREQLQQAIDAACAEVPAEIRPVIGLRNSRDSYVLVGRPDDNARVVKVIEAMAAKDKKAIEDKLRGGSAFSPRITPLKVQAAFHHPAMNMAVEQTVAWATTAGLDVELTREIAADVLVNPVDWVARVNEAYEAGARWFLDVGPDGGIVKLTANILEGRGADSFYVGDAAGQAKIFDAGMAPELPVDYQEFAPRVEHVDGTPRLVTKFTELTGRTPMMLAGMTPTTVDPAIVAAAANGGHWAELAGGGQVTPELLETHIAQLTDMLEPGINAQFNSMFLDPYLWKMQIGGKRLVPKARANGASIDGIVITAGIPEKDEAVALVKELMRDGFPWIAFKPGAIKQVNSVLAIAKEVPELPIIIQIEGGVAGGHHSWEDLDELLIATYGKVRALDNVVLCVGGGIGSPERAADYVTGSWSTSYGLPAMPVDGILVGTAAMATKEATTSQAVKELLVSTQGSDEWVPAGGAKNGMASGRSQLGADIHEIDNSFAKAGRLLDEVAGDETAVQARRDEIIEAIGKTAKVYFGDIGSMTYEQWLNRYLELSGPVDGQWIDASWAARFAQMLERAEARLIEQDHGQFEPSLTVEDGVDKLVAAYPHAATDLLTPADVAWFLGLCRTPGKPVNFVPVIDKDVRRWWRSDSLWQSHDDRYTADQVAIIPGVVAVAGITKANEPVADLLDRFVDATIERIDEHDSRSRDIMGKVLSSPGTFWAGRNIPSVIHSLGHADKWSRSEFEAFHSPTGANLVYEDAEHAMLTVPLAGSTAFGTTAELKIRFTSPIDALPSAVPLVTQEDAEAAMGELTRIAAGGTLATVNNGTATWETSVDAGVIADYNNVTAGYLPASVVPAHTAPDVLVGRAWPAVFAAVKSAVIPGTDSASVVEGMLSLVHLEHHIVLKSDVPTDGALKVSATADEVVDTDLGRLVIVRAEIADAEGNLIATLAERFAIRGRKGNAVARTNTSALPTTVDTPRSARAVATVVAPESMRPFAVISGDRNPIHVSDVAASLAGLPGVIVHGMWTSAIGELIAGAAFNDEQIQTPAAKVVEYTATMLAPVLPGEEIEFSVERSAVDNRPGMGEVRTVTATVNGNLVLTATAVVAAPSTFYAFPGQGIQSQGMGMEARRNSQAARAIWDRADAHTRNKLGFSIVEIVENNPREVTVAGEKFFHPDGVLYLTQFTQVDMATLGVAQIAEMREAHALNQRAYFAGHSVGEYNALAAYAGVLSLESVLEIVYRRGLTMHRLVDRDENGLSNYALAALRPNKMGLTADNVFDYVASVSEASGEFLEIVNYNLAGLQYAVAGTQAGLAALRADVENRAPGQRAFILIPGIDVPFHSSKLRDGVGAFREHLDSLIPAELDLDVLVGRYIPNLVARPFELTEEFVASMAEVVESTYVNEILADFKAASADKQKLARTLLIELLAWQFASPVRWIETQDLLIKGLQAERFVEVGVGSAPTLANMMGQTLRLPQYADATIEVLNIERDRPVVFATDEVVREVAVEETPAAPAETTETPATPATPAPVAAAAPATGGPRPDDISFTPSDATEMLIAIWTKVRPDQMGATDSIETLVEGVSSRRNQLLLDLGVEFGLGAIDGAADAELGDLKVTVSKMAKGYKAFGPVLSDAAADALRRLTGPTGKRPGYIAERVTGTWELGQGWADHVVAEVVIGAREGASLRGGDLASLSPASPASASDLDSLIDAAVQAVASRRGVAVSLPSAGGAAGGVVDSAALGEFAEQVTGHDGVLAQAARTILTQLGLDKPATVSVEDTAEEDLYELVSKELGSDWPRQVAPSFDEEKVVLLDDRWASAREDLSRVALGELAATDIDVTGAGEAVAAQAEFFGLDDLAAQARDQSSLDYADDVAVVTGGSPNSIAAAVVEKLLAGGATVIATTSNLGHDRLEFYKDLYARSARGTAALWIVAANLSSYSDIDAIINWVGSEQTTTVNGASKLVKPALVPTLLFPFAAPRVSGSMADAGPQAESQMRLLLWSVERLIAGLAPLGSSINVGHRLHVVIPGSPNRGRFGGDGAYGESKAALDAVVTRWNAEQAAWGAHTSLVHAHIGWVRGTGLMGGNDPLVKAAEEAGVETYSTQEIAEKLLSQATSTVREQAASAPITVDFTGGLGESDLNLAEMARAEAAKAANAPVVEAPRTVAALPTPYRPVVQTTPDFAGQVTQNLDEMVVIVGAGELGPLGSARTRFDAELNGSLSAAGVIELAWTMGLIHWDEDPKPGWYDDSDDAVAEEDIFDRYHDEVMARVGVRKYNDMPEYGMIDNFAPELTTVYLDQDLTFNVGSREEALTYVDSEPELTFASFDEAAGEWKVTRKAGSAIRVPRRMAMTRFVGGQVPKDFDPAVWGIPADMVDNLDTVALWNIVCTVDAFLSAGFTPAELLASVHPARVSSTQGTGMGGMESLRGIYVDRILAEPRANDVLQEALPNVVAAHVMQSYVGGYGQMIHPVAACATAAVSVEEALDKIRIGKSDFVVAGGFDALSVEGITGFGDMAATADSAEMEGKGIEHRFFSRANDRRRGGFIESEGGGTVLLARGSLAADLGLPVLGVIGFAESFADGAHTSIPAPGLGALGAARDGVESRLAVALRSVGVSADEISIISKHDTSTNANDPNESDLHERIASAIGRADGNPMYVISQKSLTGHAKGGAAAFQMIGLTQVLRSGLVPANRALDCVDPVLSKHSHLVWLRKPLDLRAKAPKAGLVTSLGFGHVSALVAIVHPDAFYEAVRVARGAEAADVWRASAIAREEAGLRTIVAGMHGGVLYERPVERNLGVHGDAAKEVEAAVLLDSRARLVDGVLRAEG

SEQ ID NO: 5 - DNA, 8991 bp, Corynebacterium glutamicum
gtgaccgaattgagcaggaacttcggggccagccgactgattaaccgctttggccaggagccttttgccttcgctttcgccggccaaggatatgactggttgaagacccttcgtgccgcggttgccgcaggtgcaggcaccaatgttagtgacatcgtcgagcgcgcaaatgcgctgcttgcactagttgcagatgatctcattggcacccttccatttggtttcgatccagtggcttgggctaacaactccgaagatccagctttcgatactgcacaatctgcagtgagcgtgccgggtatctttgtctcccagatcgcaaccctggattcccttgaggcgcagcgccttgatgtggatcaggctgtgtccagcattggtcattcccagggcgtattgggcgtgcacctgctcaatgatgcgactcgtgctgatgaactcgttgccattgcgcagttgatcggtgcagcgatcacccgcaccgcacgcatgacgggcctgatcgcgcagggcgacaacatgccgatgctgtcgatcgccggaatttcccgcgaacagcttcagcaagctatcgacgcggcctgcgccgaagtccctgcggagatccgcccggttatcggtctgcgcaactcacgcgattcttatgttttggttggccgcccagacgacaacgctcgcgttgttaaggtcattgaggcaatggctgccaaggataagaaggccattgaagataagctgcgcggcggttccgcgttcagcccccgtattactccgctgaaggtgcaggctgctttccatcacccagctatgaacatggctgtggagcagaccgtggcgtgggcaaccactgctggtttggatgtggaactcacccgcgagatcgccgctgatgttttggttaaccctgtcgattgggtagcacgcgtcaacgaagcgtatgaggctggcgctcgctggttcctcgacgttggaccagatggtggcatcgttaagctgactgccaacatccttgagggccgcggcgcggattccttctatgttggtgacgccgcaggccaggccaagatatttgatgctggcatggcacctgaacttccagtggattaccaggagttcgcaccacgcgttgagcacgttgatggaaccccacgcctggttaccaagttcactgagctgaccggccgcaccccaatgatgctggctggcatgaccccaaccaccgttgaccctgccattgttgcagccgctgcaaacggtggacactgggctgagctcgctggtggcggacaggttaccccagagctgctggaaacccacatcgcacagctcaccgacatgcttgagccaggtatcaacgcccagttcaactccatgttcttggatccatacctgtggaagatgcagattggtggcaagcgccttgttcctaaggcccgcgctaatggtgcatccatcgacggcatcgtcatcaccgccggcattcctgaaaaggatgaagctgttgcattggtcaaggaactgatgcgtgatggtttcccttggatcgcattcaagccaggtgccatcaagcaggttaactctgtgttggctatcgctaaggaagttccagaactccccatcatcattcagattgagggtggcgttgcaggtggacaccactcttgggaagacctcgatgagctgctgatcgccacctacggcaaggtccgcgcactggataacgtggtgctgtgtgtcggcggtggcattggctcacctgagcgcgctgctgattacgtcaccggttcctggtccacttcctacggcctgccagctatgcctgttgatggcatcttggtgggtaccgctgcgatggcaaccaaggaagcaaccacctcccaggccgtcaaggaacttcttgtttccacccagggctctgatgaatgggttcctgctggtggcgcaaagaacggaatggcatctggccgttcccagcttggcgcagacatccacgagatcgacaactcctttgctaaggctggacgccttcttgatgaggttgcaggcgatgagacggctgtgcaggcgcgccgggatgagatcattgaagcgattggcaagaccgccaaggtgtacttcggtgacatcggatccatgacttacgagcagtggctcaaccgctacctcgagctgtctggccctgttgatggtcagtggattgatgcttcctgggctgcacgttttgcccagatgctggagcgtgccgaggcgcgtttgatcgagcaggatcatggccaatttgagccaagcctgacggtggaggatggcgtcgacaagcttgttgctgcttacccgcatgccgcaaccgacctgctcaccccggctgatgtcgcctggttcttgggcctgtgccgcacgccgggcaagcctgtgaactttgtgcccgtcattgataaggacgtgcgtcgctggtggcgctcggactccctgtggcagtcccacgatgatcgctacaccgctgatcaggtggctattatccctggtgtcgtcgccgttgctggcatcaccaaggccaacgaacctgtcgctgacctgcttgatcgctttgtcgacgccaccatcgagcgcatcgatgagcacgattcccgctcccgcgacatcatgggcaaagtgctttcctcacctggcacattctgggctggccgcaacatcccatcggtgatccacagccttgggcatgctgacaagtggtcccgctccgaattcgaagcattccatagcccaaccggcgccaacttggtgtacgaagacgccgagcacgcgatgctgactgtgcctttggcgggttccaccgcattcggcaccaccgctgagctgaaaatccgtttcaccagccccatcgacgctctgccaagcgctgtcccactggtcacccaggaagacgctgaagccgcgatgggtgaactgacccgcatcgcagctggcggcaccctggcaactgtgaacaatggcaccgctacctgggaaacctccgtcgatgccggcgtcatcgctgactacaacaacgtcaccgcaggctacctgccagcatccgttgttcctgcacacaccgcacctgacgtgctggttggccgcgcatggccagcagttttcgctgccgtaaagtccgcagtcatcccaggcaccgattccgcatccgttgtggaaggcatgctgtccctggttcacctggagcaccacattgtgctcaagtccgatgtcccaaccgacggcgcgctgaaggtttccgcgactgccgatgaggtagtcgataccgacctgggtcgcctcgtgatcgtgcgcgcagaaatcgccgacgcagaaggcaacctgattgctacgttggctgagcgtttcgcgatccgcggacgcaagggcaacgctgtcgcacgcaccaacacctccgcactgccaaccaccgtggacaccccacgctcagctcgcgcagtggcaaccgttgttgcacctgaatccatgcgcccattcgctgtgatctccggtgaccgcaacccaattcacgtctctgatgttgcggcttccctggctggtctgccaggtgtgatcgtgcacggcatgtggacctctgccatcggtgaactgatcgccggtgcagcattcaacgatgagcagatccaaactcccgcagccaaggtcgtggaatacaccgcaaccatgctggcaccagttcttccaggtgaagaaattgagttcagcgttgagcgctccgcagtggacaaccgcccaggaatgggagaggtccgcaccgttaccgcaaccgtcaacggcaacttagtgcttaccgccaccgctgttgtggcagctccatctactttctacgcattcccaggccagggcattcagtcccagggcatgggtatggaagcacgccgtaactctcaggcagctcgcgctatctgggaccgcgccgatgcacacacccgcaataagctgggcttctccatcgtggaaatcgtggaaaacaacccacgcgaagtaaccgtggcaggggagaagttcttccacccagacggcgttttgtacctcacccagttcacccaggtgggcatggcaactctgggcgttgctcagatcgctgaaatgcgtgaagcacatgccttgaaccagcgtgcatactttgctggacactccgttggtgagtacaacgcgcttgctgcatatgctggtgtgctgtccctggaatccgttctggagatcgtttaccgtcgtggcttgaccatgcaccgcttggtggatcgcgatgaaaacggtctgtccaactacgcgctcgcagctcttcgccccaacaagatgggtctgaccgcagacaacgttttcgattacgttgcgtctgtttccgaagcttccggtgaattcctggagatcgttaactacaacttggctggcctgcagtacgcagttgctggaacccaggctggtcttgccgcccttcgtgccgatgttgagaaccgtgcaccaggtcagcgtgccttcattttgatccctggcattgacgtgccattccactcctccaagctgcgcgacggtgtgggcgcgttccgtgagcaccttgattccctgatcccagctgagctggatctggatgtgctggttggccgctacattccaaacttggtggctcgcccattcgaactcactgaagagttcgtggcatccatggcagaagtggtggagtccacctatgtcaatgagatcttggctgatttcaaggctgcttccgccgataagcagaagcttgcccgcacgttgcttattgagctgcttgcatggcagttcgcatcacctgtgcgctggatcgagactcaggatctgttgatcaagggccttcaagctgagcgtttcgtggaggtcggtgttggctctgctccaacgcttgccaacatgatgggccagaccctgcgccttcctcagtacgcggacgccaccattgaggtgttaaacattgagcgcgatcgcccagttgtgttcgctaccgatgaggttgtgcgtgaagtggcggttgaagagaccccagcagctcctgcagaaaccactgaaaccccagcaaccccagcaaccccagcccctgttgcagctgcagcccctgccaccggcggccctcgcccagatgacatcagcttcactccttctgatgccactgaaatgctcatcgctatctggaccaaggttcgcccagatcagatgggtgccactgattccatcgagaccctggttgagggcgtgtcctctcgccgtaaccagctcctgctggatcttggtgtggagttcggcctcggcgcaattgacggagcagccgatgctgagctcggtgatctaaaggtcaccgtgtccaagatggctaagggctacaaggcgtttggccctgtgctctccgatgctgcagctgatgccctgcgtcgcctcactggtcctaccggtaagcgcccgggatacatcgcagagcgcgtcaccggcacgtgggaattgggccagggctgggctgaccacgtggtcgctgaagttgtgatcggcgcccgcgaaggcgcatccctgcgcggcggcgacctggcgtcactgtctcctgcaagcccagcgtctgcatcagatcttgattcgcttatcgacgcagccgtccaggccgtagcctcccgccgcggcgttgcggtctccctgccttcagcaggcggcgctgccggtggcgtggttgattccgcagctcttggcgagtttgcagagcaggtcaccggacacgatggtgtgcttgctcaggcagcccgcaccatcttgacccagttgggtcttgataagccagcaaccgtttccgtggaagacaccgcagaggaagacctctacgagttggtctccaaggaactcggttctgattggccacgtcaggttgcaccaagcttcgatgaagaaaaggttgttctgcttgatgaccgttgggcttctgcgcgtgaggatctctcccgcgttgctcttggcgaactcgcagcaactgatatcgatgtcacaggcgcaggcgaagctgttgcagcacaggctgaattctttggacttgatgatctcgcagctcaggctcgcgaccaaagctccttggactacgccgacgatgttgcggtcgtaaccggcggatcgccgaactcgatcgcggcggcagtcgtcgaaaagcttcttgctggcggtgcaactgtcattgctacgacctccaacctcggccatgaccgcctggagttctacaaggatctctacgcacgttccgcacgcggcacggcagcactgtggatcgtggcggctaacttgagctcctactcagacatcgacgccatcatcaactgggtcggatccgagcagaccaccaccgtcaacggcgcatccaagctggtcaagccagctttggtccctaccttgctgttcccattcgcggcacctcgcgtgtccgaatccatggcagatgcaggcccacaggcagaatcccagatgcgacttctgctctggtctgttgagcgcctcatcgcaggtcttgcgccattgggctcctccatcaacgtgggtcaccgcctgcacgtggtcatcccaggttcaccaaaccgtggacgcttcggtggcgatggtgcatacggtgaatccaaggcagctctcgacgccgtggttacccgttggaacgcagagcaagctgcatggggagcacacacctccctcgtgcacgctcacatcggttgggttcgcggcaccggcctcatgggcggcaacgatcctttggtcaaggcagctgaagaagcaggcgtggaaacctactccacccaagaaattgcagagaaactgctgtcccaggcaacttccactgttcgcgagcaggcagcatccgcgccaatcaccgtcgacttcactggcggacttggtgaatctgatctgaacctggcggaaatggcacgtgcagaagcagctaaggcagctaacgcaccagtggttgaggctccacgcacagtggcagcactgccaactccttaccgaccagtggttcaaaccacccctgatttcgcaggtcaagtcacccaaaaccttgacgagatggtcgtcatcgttggcgccggcgagctcggcccactgggttctgcacgtacgcgtttcgacgccgaactcaacggttccctctccgccgcgggtgtcatcgaacttgcatggacgatgggacttatccactgggatgaagatccaaagccaggctggtacgacgactccgacgacgcagtggccgaagaagacatcttcgaccgctaccacgacgaagtcatggcacgcgttggtgtccgcaagtacaatgacatgcctgagtacggcatgatcgacaactttgcaccagagctgaccaccgtctacctcgaccaggacctcaccttcaacgtgggatcccgcgaagaggcactgacctacgtcgactccgagccagaactcacctttgcttctttcgacgaagcagcaggggagtggaaggtcactcgcaaggcaggctccgcaatccgcgtacctcgccgcatggcgatgacccgcttcgttggtggacaggttcctaaggacttcgacccagctgtgtggggcattccagctgacatggtggacaacctggacaccgtcgcgctgtggaacattgtctgtactgtcgacgccttcctgtccgctggattcaccccagcagagctgcttgcttccgttcacccagcacgcgtgtcctctacccaaggcaccggcatgggcggcatggaatccctccgtggcatctacgtcgaccgcattctggcagagccacgcgccaacgacgttctgcaggaagcactgcccaacgttgttgcagctcacgtcatgcagtcctacgtcggtggctacggacagatgatccacccagtcgcagcttgtgcaaccgcagctgtttctgtggaagaagcactggacaagatccgcatcggcaagtccgacttcgttgtcgcaggtggcttcgatgccctgtccgttgaaggcatcaccggcttcggcgacatggcagcaaccgccgactccgcagagatggaaggcaagggaattgagcaccgcttcttctcccgcgccaacgaccgccgccgcggtggattcatcgaatccgaaggtggcggaaccgtccttctggcacgcggatcactcgcagctgacctgggccttccagtactcggtgtcatcggattcgcagagtcctttgcagatggtgcccacacctccatcccagccccaggcctcggtgcccttggtgctgctcgcgatggtgtggaatctcgccttgcagtagcactgcgttccgtcggtgtctctgctgatgagatctccattatctccaagcacgacacctccaccaacgcgaatgatccaaacgagtccgacctgcacgagcgcatcgcatccgctatcggtcgtgcagacggcaacccgatgtacgtgatttcccagaagtcactcaccggacacgccaagggtggtgcagcagcattccagatgatcggtctcacccaggtcctccgatccggactggtgccagccaaccgcgcactcgactgcgttgacccagtactgtccaagcattcccacctcgtctggctgcgcaagccactagaccttcgtgcgaaggcaccaaaggcaggtcttgttacctcccttggcttcggacacgtctccgctctggttgcgattgttcacccagacgccttctatgaggcagttcgtgtggcacgtggtgctgaggcagctgacgtatggcgcgcatccgcgatcgctcgcgaagaagcaggccttcgtaccatcgtcgccggtatgcacggtggcgtactgtacgaacgcccagtcgagcgcaacctcggtgtccacggagacgcagctaaggaagttgaagctgcagtcctcctggattcccgcgcccgcctagttgacggtgtcctccgcgccgaaggctag

SEQ ID NO: 6 - protein, 2996 aa, Corynebacterium glutamicum
MTELSRNFGASRLINRFGQEPFAFAFAGQGYDWLKTLRAAVAAGAGTNVSDIVERANALLALVADDLIGTLPFGFDPVAWANNSEDPAFDTAQSAVSVPGIFVSQIATLDSLEAQRLDVDQAVSSIGHSQGVLGVHLLNDATRADELVAIAQLIGAAITRTARMTGLIAQGDNMPMLSIAGISREQLQQAIDAACAEVPAEIRPVIGLRNSRDSYVLVGRPDDNARVVKVIEAMAAKDKKAIEDKLRGGSAFSPRITPLKVQAAFHHPAMNMAVEQTVAWATTAGLDVELTREIAADVLVNPVDWVARVNEAYEAGARWFLDVGPDGGIVKLTANILEGRGADSFYVGDAAGQAKIFDAGMAPELPVDYQEFAPRVEHVDGTPRLVTKFTELTGRTPMMLAGMTPTTVDPAIVAAAANGGHWAELAGGGQVTPELLETHIAQLTDMLEPGINAQFNSMFLDPYLWKMQIGGKRLVPKARANGASIDGIVITAGIPEKDEAVALVKELMRDGFPWIAFKPGAIKQVNSVLAIAKEVPELPIIIQIEGGVAGGHHSWEDLDELLIATYGKVRALDNVVLCVGGGIGSPERAADYVTGSWSTSYGLPAMPVDGILVGTAAMATKEATTSQAVKELLVSTQGSDEWVPAGGAKNGMASGRSQLGADIHEIDNSFAKAGRLLDEVAGDETAVQARRDEIIEAIGKTAKVYFGDIGSMTYEQWLNRYLELSGPVDGQWIDASWAARFAQMLERAEARLIEQDHGQFEPSLTVEDGVDKLVAAYPHAATDLLTPADVAWFLGLCRTPGKPVNFVPVIDKDVRRWWRSDSLWQSHDDRYTADQVAIIPGVVAVAGITKANEPVADLLDRFVDATIERIDEHDSRSRDIMGKVLSSPGTFWAGRNIPSVIHSLGHADKWSRSEFEAFHSPTGANLVYEDAEHAMLTVPLAGSTAFGTTAELKIRFTSPIDALPSAVPLVTQEDAEAAMGELTRIAAGGTLATVNNGTATWETSVDAGVIADYNNVTAGYLPASVVPAHTAPDVLVGRAWPAVFAAVKSAVIPGTDSASVVEGMLSLVHLEHHIVLKSDVPTDGALKVSATADEVVDTDLGRLVIVRAEIADAEGNLIATLAERFAIRGRKGNAVARTNTSALPTTVDTPRSARAVATVVAPESMRPFAVISGDRNPIHVSDVAASLAGLPGVIVHGMWTSAIGELIAGAAFNDEQIQTPAAKVVEYTATMLAPVLPGEEIEFSVERSAVDNRPGMGEVRTVTATVNGNLVLTATAVVAAPSTFYAFPGQGIQSQGMGMEARRNSQAARAIWDRADAHTRNKLGFSIVEIVENNPREVTVAGEKFFHPDGVLYLTQFTQVGMATLGVAQIAEMREAHALNQRAYFAGHSVGEYNALAAYAGVLSLESVLEIVYRRGLTMHRLVDRDENGLSNYALAALRPNKMGLTADNVFDYVASVSEASGEFLEIVNYNLAGLQYAVAGTQAGLAALRADVENRAPGQRAFILIPGIDVPFHSSKLRDGVGAFREHLDSLIPAELDLDVLVGRYIPNLVARPFELTEEFVASMAEVVESTYVNEILADFKAASADKQKLARTLLIELLAWQFASPVRWIETQDLLIKGLQAERFVEVGVGSAPTLANMMGQTLRLPQYADATIEVLNIERDRPVVFATDEVVREVAVEETPAAPAETTETPATPATPAPVAAAAPATGGPRPDDISFTPSDATEMLIAIWTKVRPDQMGATDSIETLVEGVSSRRNQLLLDLGVEFGLGAIDGAADAELGDLKVTVSKMAKGYKAFGPVLSDAAADALRRLTGPTGKRPGYIAERVTGTWELGQGWADHVVAEVVIGAREGASLRGGDLASLSPASPASASDLDSLIDAAVQAVASRRGVAVSLPSAGGAAGGVVDSAALGEFAEQVTGHDGVLAQAARTILTQLGLDKPATVSVEDTAEEDLYELVSKELGSDWPRQVAPSFDEEKVVLLDDRWASAREDLSRVALGELAATDIDVTGAGEAVAAQAEFFGLDDLAAQARDQSSLDYADDVAVVTGGSPNSIAAAVVEKLLAGGATVIATTSNLGHDRLEFYKDLYARSARGTAALWIVAANLSSYSDIDAIINWVGSEQTTTVNGASKLVKPALVPTLLFPFAAPRVSESMADAGPQAESQMRLLLWSVERLIAGLAPLGSSINVGHRLHVVIPGSPNRGRFGGDGAYGESKAALDAVVTRWNAEQAAWGAHTSLVHAHIGWVRGTGLMGGNDPLVKAAEEAGVETYSTQEIAEKLLSQATSTVREQAASAPITVDFTGGLGESDLNLAEMARAEAAKAANAPVVEAPRTVAALPTPYRPVVQTTPDFAGQVTQNLDEMVVIVGAGELGPLGSARTRFDAELNGSLSAAGVIELAWTMGLIHWDEDPKPGWYDDSDDAVAEEDIFDRYHDEVMARVGVRKYNDMPEYGMIDNFAPELTTVYLDQDLTFNVGSREEALTYVDSEPELTFASFDEAAGEWKVTRKAGSAIRVPRRMAMTRFVGGQVPKDFDPAVWGIPADMVDNLDTVALWNIVCTVDAFLSAGFTPAELLASVHPARVSSTQGTGMGGMESLRGIYVDRILAEPRANDVLQEALPNVVAAHVMQSYVGGYGQMIHPVAACATAAVSVEEALDKIRIGKSDFVVAGGFDALSVEGITGFGDMAATADSAEMEGKGIEHRFFSRANDRRRGGFIESEGGGTVLLARGSLAADLGLPVLGVIGFAESFADGAHTSIPAPGLGALGAARDGVESRLAVALRSVGVSADEISIISKHDTSTNANDPNESDLHERIASAIGRADGNPMYVISQKSLTGHAKGGAAAFQMIGLTQVLRSGLVPANRALDCVDPVLSKHSHLVWLRKPLDLRAKAPKAGLVTSLGFGHVSALVAIVHPDAFYEAVRVARGAEAADVWRASAIAREEAGLRTIVAGMHGGVLYERPVERNLGVHGDAAKEVEAAVLLDSRARLVDGVLRAEG

SEQ ID NO: 7 - DNA, 8991 bp, Corynebacterium glutamicum
gtgaccgaattgagcaggaacttcggggccagccgactgattaaccgctttggccaggagccttttgccttcgctttcgccggccaaggatatgactggttgaagacccttcgtgccgcggttgccgcaggtgcaggcaccaatgttagtgacatcgtcgagcgcgcaaatgcgctgcttgcactagttgcagatgatctcattggcacccttccatttggtttcgatccagtggcttgggctaacaactccgaagatccagctttcgatactgcacaatctgcagtgagcgtgccgggtatctttgtctcccagatcgcaaccctggattcccttgaggcgcagcgccttgatgtggatcaggctgtgtccagcattggtcattcccagggcgtattgggcgtgcacctgctcaatgatgcgactcgtgctgatgaactcgttgccattgcgcagttgatcggtgcagcgatcacccgcaccgcacgcatgacgggcctgatcgcgcagggcgacaacatgccgatgctgtcgatcgccggaatttcccgcgaacagcttcagcaagctatcgacgcggcctgcgccgaagtccctgcggagatccgcccggttatcggtctgcgcaactcacgcgattcttatgttttggttggccgcccagacgacaacgctcgcgttgttaaggtcattgaggcaatggctgccaaggataagaaggccattgaagataagctgcgcggcggttccgcgttcagcccccgtattactccgctgaaggtgcaggctgctttccatcacccagctatgaacatggctgtggagcagaccgtggcgtgggcaaccactgctggtttggatgtggaactcacccgcgagatcgccgctgatgttttggttaaccctgtcgattgggtagcacgcgtcaacgaagcgtatgaggctggcgctcgctggttcctcgacgttggaccagatggtggcatcgttaagctgactgccaacatccttgagggccgcggcgcggattccttctatgttggtgacgccgcaggccaggccaagatatttgatgctggcatggcacctgaacttccagtggattaccaggagttcgcaccacgcgttgagcacgttgatggaaccccacgcctggttaccaagttcactgagctgaccggccgcaccccaatgatgctggctggcatgaccccaaccaccgttgaccctgccattgttgcagccgctgcaaacggtggacactgggctgagctcgctggtggcggacaggttaccccagagctgctggaaacccacatcgcacagctcaccgacatgcttgagccaggtatcaacgcccagttcaactccatgttcttggatccatacctgtggaagatgcagattggtggcaagcgccttgttcctaaggcccgcgctaatggtgcatccatcgacggcatcgtcatcaccgccggcattcctgaaaaggatgaagctgttgcattggtcaaggaactgatgcgtgatggtttcccttggatcgcattcaagccaggtgccatcaagcaggttaactctgtgttggctatcgctaaggaagttccagaactccccatcatcattcagattgagggtggcgttgcaggtggacaccactcttgggaagacctcgatgagctgctgatcgccacctacggcaaggtccgcgcactggataacgtggtgctgtgtgtcggcggtggcattggctcacctgagcgcgctgctgattacgtcaccggttcctggtccacttcctacggcctgccagctatgcctgttgatggcatcttggtgggtaccgctgcgatggcaaccaaggaagcaaccacctcccaggccgtcaaggaacttcttgtttccacccagggctctgatgaatgggttcctgctggtggcgcaaagaacggaatggcatctggccgttcccagcttggcgcagacatccacgagatcgacaactcctttgctaaggctggacgccttcttgatgaggttgcaggcgatgagacggctgtgcaggcgcgccgggatgagatcattgaagcgattggcaagaccgccaaggtgtacttcggtgacatcggatccatgacttacgagcagtggctcaaccgctacctcgagctgtctggccctgttgatggtcagtggattgatgcttcctgggctgcacgttttgcccagatgctggagcgtgccgaggcgcgtttgatcgagcaggatcatggccaatttgagccaagcctgacggtggaggatggcgtcgacaagcttgttgctgcttacccgcatgccgcaaccgacctgctcaccccggctgatgtcgcctggttcttgggcctgtgccgcacgccgggcaagcctgtgaactttgtgcccgtcattgataaggacgtgcgtcgctggtggcgctcggactccctgtggcagtcccacgatgatcgctacaccgctgatcaggtggctattatccctggtgtcgtcgccgttgctggcatcaccaaggccaacgaacctgtcgctgacctgcttgatcgctttgtcgacgccaccatcgagcgcatcgatgagcacgattcccgctcccgcgacatcatgggcaaagtgctttcctcacctggcacattctgggctggccgcaacatcccatcggtgatccacagccttgggcatgctgacaagtggtcccgctccgaattcgaagcattccatagcccaaccggcgccaacttggtgtacgaagacgccgagcacgcgatgctgactgtgcctttggcgggttccaccgcattcggcaccaccgctgagctgaaaatccgtttcaccagccccatcgacgctctgccaagcgctgtcccactggtcacccaggaagacgctgaagccgcgatgggtgaactgacccgcatcgcagctggcggcaccctggcaactgtgaacaatggcaccgctacctgggaaacctccgtcgatgccggcgtcatcgctgactacaacaacgtcaccgcaggctacctgccagcatccgttgttcctgcacacaccgcacctgacgtgctggttggccgcgcatggccagcagttttcgctgccgtaaagtccgcagtcatcccaggcaccgattccgcatccgttgtggaaggcatgctgtccctggttcacctggagcaccacattgtgctcaagtccgatgtcccaaccgacggcgcgctgaaggtttccgcgactgccgatgaggtagtcgataccgacctgggtcgcctcgtgatcgtgcgcgcagaaatcgccgacgcagaaggcaacctgattgctacgttggctgagcgtttcgcgatccgcggacgcaagggcaacgctgtcgcacgcaccaacacctccgcactgccaaccaccgtggacaccccacgctcagctcgcgcagtggcaaccgttgttgcacctgaatccatgcgcccattcgctgtgatctccggtgaccgcaacccaattcacgtctctgatgttgcggcttccctggctggtctgccaggtgtgatcgtgcacggcatgtggacctctgccatcggtgaactgatcgccggtgcagcattcaacgatgagcagatccaaactcccgcagccaaggtcgtggaatacaccgcaaccatgctggcaccagttcttccaggtgaagaaattgagttcagcgttgagcgctccgcagtggacaaccgcccaggaatgggagaggtccgcaccgttaccgcaaccgtcaacggcaacttagtgcttaccgccaccgctgttgtggcagctccatctactttctacgcattcccaggccagggcattcagtcccagggcatgggtatggaagcacgccgtaactctcaggcagctcgcgctatctgggaccgcgccgatgcacacacccgcaataagctgggcttctccatcgtggaaatcgtggaaaacaacccacgcgaagtaaccgtggcaggggagaagttcttccacccagacggcgttttgtacctcacccagttcacccaggtgggcatggcaactctgggcgttgctcagatcgctgaaatgcgtgaagcacatgccttgaaccagcgtgcatactttgctggacactccgttggtgagtacaacgcgcttgctgcatatgctggtgtgctgtccctggaatccgttctggagatcgtttaccgtcgtggcttgaccatgcaccgcttggtggatcgcgatgaaaacggtctgtccaactacgcgctcgcagctcttcgccccaacaagatgggtctgaccgcagacaacgttttcgattacgttgcgtctgtttccgaagcttccggtgaattcctggagatcgttaactacaacttggctggcctgcagtacgcagttgctggaacccaggctggtcttgccgcccttcgtgccgatgttgagaaccgtgcaccaggtcagcgtgccttcattttgatccctggcattgacgtgccattccactcctccaagctgcgcgacggtgtgggcgcgttccgtgagcaccttgattccctgatcccagctgagctggatctggatgtgctggttggccgctacattccaaacttggtggctcgcccattcgaactcactgaagagttcgtggcatccatggcagaagtggtggagtccacctatgtcaatgagatcttggctgatttcaaggctgcttccgccgataagcagaagcttgcccgcacgttgcttattgagctgcttgcatggcagttcgcatcacctgtgcgctggatcgagactcaggatctgttgatcaagggccttcaagctgagcgtttcgtggaggtcggtgttggctctgctccaacgcttgccaacatgatgggccagaccctgcgccttcctcagtacgcggacgccaccattgaggtgttaaacattgagcgcgatcgcccagttgtgttcgctaccgatgaggttgtgcgtgaagtggcggttgaagagaccccagcagctcctgcagaaaccactgaaaccccagcaaccccagcaaccccagcccctgttgcagctgcagcccctgccaccggcggccctcgcccagatgacatcagcttcactccttctgatgccactgaaatgctcatcgctatctggaccaaggttcgcccagatcagatgggtgccactgattccatcgagaccctggttgagggcgtgtcctctcgccgtaaccagctcctgctggatcttggtgtggagttcggcctcggcgcaattgacggagcagccgatgctgagctcggtgatctaaaggtcaccgtgtccaagatggctaagggctacaaggcgtttggccctgtgctctccgatgctgcagctgatgccctgcgtcgcctcactggtcctaccggtaagcgcccgggatacatcgcagagcgcgtcaccggcacgtgggaattgggccagggctgggctgaccacgtggtcgctgaagttgtgatcggcgcccgcgaaggcgcatccctgcgcggcggcgacctggcgtcactgtctcctgcaagcccagcgtctgcatcagatcttgattcgcttatcgacgcagccgtccaggccgtagcctcccgccgcggcgttgcggtctccctgccttcagcaggcggcgctgccggtggcgtggttgattccgcagctcttggcgagtttgcagagcaggtcaccggacacgatggtgtgcttgctcaggcagcccgcaccatcttgacccagttgggtcttgataagccagcaaccgtttccgtggaagacaccgcagaggaagacctctacgagttggtctccaaggaactcggttctgattggccacgtcaggttgcaccaagcttcgatgaagaaaaggttgttctgcttgatgaccgttgggcttctgcgcgtgaggatctctcccgcgttgctcttggcgaactcgcagcaactgatatcgatgtcacaggcgcaggcgaagctgttgcagcacaggctgaattctttggacttgatgatctcgcagctcaggctcgcgaccaaagctccttggactacgccgacgatgttgcggtcgtaaccggcggatcgccgaactcgatcgcggcggcagtcgtcgaaaagcttcttgctggcggtgcaactgtcattgctacgacctccaacctcggccatgaccgcctggagttctacaaggatctctacgcacgttccgcacgcggcacggcagcactgtggatcgtggcggctaacttgagctcctactcagacatcgacgccatcatcaactgggtcggatccgagcagaccaccaccgtcaacggcgcatccaagctggtcaagccagctttggtccctaccttgctgttcccattcgcggcacctcgcgtgtccggatccatggcagatgcaggcccacaggcagaatcccagatgcgacttctgctctggtctgttgagcgcctcatcgcaggtcttgcgccattgggctcctccatcaacgtgggtcaccgcctgcacgtggtcatcccaggttcaccaaaccgtggacgcttcggtggcgatggtgcatacggtgaatccaaggcagctctcgacgccgtggttacccgttggaacgcagagcaagctgcatggggagcacacacctccctcgtgcacgctcacatcggttgggttcgcggcaccggcctcatgggcggcaacgatcctttggtcaaggcagctgaagaagcaggcgtggaaacctactccacccaagaaattgcagagaaactgctgtcccaggcaacttccactgttcgcgagcaggcagcatccgcgccaatcaccgtcgacttcactggcggacttggtgaatctgatctgaacctggcggaaatggcacgtgcagaagcagctaaggcagctaacgcaccagtggttgaggctccacgcacagtggcagcactgccaactccttaccgaccagtggttcaaaccacccctgatttcgcaggtcaagtcacccaaaaccttgacgagatggtcgtcatcgttggcgccggcgagctcggcccactgggttctgcacgtacgcgtttcgacgccgaactcaacggttccctctccgccgcgggtgtcatcgaacttgcatggacgatgggacttatccactgggatgaagatccaaagccaggctggtacgacgactccgacgacgcagtggccgaagaagacatcttcgaccgctaccacgacgaagtcatggcacgcgttggtgtccgcaagtacaatgacatgcctgagtacggcatgatcgacaactttgcaccagagctgaccaccgtctacctcgaccaggacctcaccttcaacgtgggatcccgcgaagaggcactgacctacgtcgactccgagccagaactcacctttgcttctttcgacgaagcagcaggggagtggaaggtcactcgcaaggcaggctccgcaatccgcgtacctcgccgcatggcgatgacccgcttcgttggtggacaggttcctaaggacttcgacccagctgtgtggggcattccagctgacatggtggacaacctggacaccgtcgcgctgtggaacattgtctgtactgtcgacgccttcctgtccgctggattcaccccagcagagctgcttgcttccgttcacccagcacgcgtgtcctctacccaaggcaccggcatgggcggcatggaatccctccgtggcatctacgtcgaccgcattctggcagagccacgcgccaacgacgttctgcaggaagcactgcccaacgttgttgcagctcacgtcatgcagtcctacgtcggtggctacggacagatgatccacccagtcgcagcttgtgcaaccgcagctgtttctgtggaagaagcactggacaagatccgcatcggcaagtccgacttcgttgtcgcattcggcttcgatgccctgtccgttgaaggcatcaccggcttcggcgacatggcagcaaccgccgactccgcagagatggaaggcaagggaattgagcaccgcttcttctcccgcgccaacgaccgccgccgcggtggattcatcgaatccgaaggtggcggaaccgtccttctggcacgcggatcactcgcagctgacctgggccttccagtactcggtgtcatcggattcgcagagtcctttgcagatggtgcccacacctccatcccagccccaggcctcggtgcccttggtgctgctcgcgatggtgtggaatctcgccttgcagtagcactgcgttccgtcggtgtctctgctgatgagatctccattatctccaagcacgacacctccaccaacgcgaatgatccaaacgagtccgacctgcacgagcgcatcgcatccgctatcggtcgtgcagacggcaacccgatgtacgtgatttcccagaagtcactcaccggacacgccaagggtggtgcagcagcattccagatgatcggtctcacccaggtcctccgatccggactggtgccagccaaccgcgcactcgactgcgttgacccagtactgtccaagcattcccacctcgtctggctgcgcaagccactagaccttcgtgcgaaggcaccaaaggcaggtcttgttacctcccttggcttcggacacgtctccgctctggttgcgattgttcacccagacgccttctatgaggcagttcgtgtggcacgtggtgctgaggcagctgacgtatggcgcgcatccgcgatcgctcgcgaagaagcaggccttcgtaccatcgtcgccggtatgcacggtggcgtactgtacgaacgcccagtcgagcgcaacctcggtgtccacggagacgcagctaaggaagttgaagctgcagtcctcctggattcccgcgcccgcctagttgacggtgtcctccgcgccgaaggctag

SEQ ID NO: 8 - protein, 2996 aa, Corynebacterium glutamicum
MTELSRNFGASRLINRFGQEPFAFAFAGQGYDWLKTLRAAVAAGAGTNVSDIVERANALLALVADDLIGTLPFGFDPVAWANNSEDPAFDTAQSAVSVPGIFVSQIATLDSLEAQRLDVDQAVSSIGHSQGVLGVHLLNDATRADELVAIAQLIGAAITRTARMTGLIAQGDNMPMLSIAGISREQLQQAIDAACAEVPAEIRPVIGLRNSRDSYVLVGRPDDNARVVKVIEAMAAKDKKAIEDKLRGGSAFSPRITPLKVQAAFHHPAMNMAVEQTVAWATTAGLDVELTREIAADVLVNPVDWVARVNEAYEAGARWFLDVGPDGGIVKLTANILEGRGADSFYVGDAAGQAKIFDAGMAPELPVDYQEFAPRVEHVDGTPRLVTKFTELTGRTPMMLAGMTPTTVDPAIVAAAANGGHWAELAGGGQVTPELLETHIAQLTDMLEPGINAQFNSMFLDPYLWKMQIGGKRLVPKARANGASIDGIVITAGIPEKDEAVALVKELMRDGFPWIAFKPGAIKQVNSVLAIAKEVPELPIIIQIEGGVAGGHHSWEDLDELLIATYGKVRALDNVVLCVGGGIGSPERAADYVTGSWSTSYGLPAMPVDGILVGTAAMATKEATTSQAVKELLVSTQGSDEWVPAGGAKNGMASGRSQLGADIHEIDNSFAKAGRLLDEVAGDETAVQARRDEIIEAIGKTAKVYFGDIGSMTYEQWLNRYLELSGPVDGQWIDASWAARFAQMLERAEARLIEQDHGQFEPSLTVEDGVDKLVAAYPHAATDLLTPADVAWFLGLCRTPGKPVNFVPVIDKDVRRWWRSDSLWQSHDDRYTADQVAIIPGVVAVAGITKANEPVADLLDRFVDATIERIDEHDSRSRDIMGKVLSSPGTFWAGRNIPSVIHSLGHADKWSRSEFEAFHSPTGANLVYEDAEHAMLTVPLAGSTAFGTTAELKIRFTSPIDALPSAVPLVTQEDAEAAMGELTRIAAGGTLATVNNGTATWETSVDAGVIADYNNVTAGYLPASVVPAHTAPDVLVGRAWPAVFAAVKSAVIPGTDSASVVEGMLSLVHLEHHIVLKSDVPTDGALKVSATADEVVDTDLGRLVIVRAEIADAEGNLIATLAERFAIRGRKGNAVARTNTSALPTTVDTPRSARAVATVVAPESMRPFAVISGDRNPIHVSDVAASLAGLPGVIVHGMWTSAIGELIAGAAFNDEQIQTPAAKVVEYTATMLAPVLPGEEIEFSVERSAVDNRPGMGEVRTVTATVNGNLVLTATAVVAAPSTFYAFPGQGIQSQGMGMEARRNSQAARAIWDRADAHTRNKLGFSIVEIVENNPREVTVAGEKFFHPDGVLYLTQFTQVGMATLGVAQIAEMREAHALNQRAYFAGHSVGEYNALAAYAGVLSLESVLEIVYRRGLTMHRLVDRDENGLSNYALAALRPNKMGLTADNVFDYVASVSEASGEFLEIVNYNLAGLQYAVAGTQAGLAALRADVENRAPGQRAFILIPGIDVPFHSSKLRDGVGAFREHLDSLIPAELDLDVLVGRYIPNLVARPFELTEEFVASMAEVVESTYVNEILADFKAASADKQKLARTLLIELLAWQFASPVRWIETQDLLIKGLQAERFVEVGVGSAPTLANMMGQTLRLPQYADATIEVLNIERDRPVVFATDEVVREVAVEETPAAPAETTETPATPATPAPVAAAAPATGGPRPDDISFTPSDATEMLIAIWTKVRPDQMGATDSIETLVEGVSSRRNQLLLDLGVEFGLGAIDGAADAELGDLKVTVSKMAKGYKAFGPVLSDAAADALRRLTGPTGKRPGYIAERVTGTWELGQGWADHVVAEVVIGAREGASLRGGDLASLSPASPASASDLDSLIDAAVQAVASRRGVAVSLPSAGGAAGGVVDSAALGEFAEQVTGHDGVLAQAARTILTQLGLDKPATVSVEDTAEEDLYELVSKELGSDWPRQVAPSFDEEKVVLLDDRWASAREDLSRVALGELAATDIDVTGAGEAVAAQAEFFGLDDLAAQARDQSSLDYADDVAVVTGGSPNSIAAAVVEKLLAGGATVIATTSNLGHDRLEFYKDLYARSARGTAALWIVAANLSSYSDIDAIINWVGSEQTTTVNGASKLVKPALVPTLLFPFAAPRVSGSMADAGPQAESQMRLLLWSVERLIAGLAPLGSSINVGHRLHVVIPGSPNRGRFGGDGAYGESKAALDAVVTRWNAEQAAWGAHTSLVHAHIGWVRGTGLMGGNDPLVKAAEEAGVETYSTQEIAEKLLSQATSTVREQAASAPITVDFTGGLGESDLNLAEMARAEAAKAANAPVVEAPRTVAALPTPYRPVVQTTPDFAGQVTQNLDEMVVIVGAGELGPLGSARTRFDAELNGSLSAAGVIELAWTMGLIHWDEDPKPGWYDDSDDAVAEEDIFDRYHDEVMARVGVRKYNDMPEYGMIDNFAPELTTVYLDQDLTFNVGSREEALTYVDSEPELTFASFDEAAGEWKVTRKAGSAIRVPRRMAMTRFVGGQVPKDFDPAVWGIPADMVDNLDTVALWNIVCTVDAFLSAGFTPAELLASVHPARVSSTQGTGMGGMESLRGIYVDRILAEPRANDVLQEALPNVVAAHVMQSYVGGYGQMIHPVAACATAAVSVEEALDKIRIGKSDFVVASGFDALSVEGITGFGDMAATADSAEMEGKGIEHRFFSRANDRRRGGFIESEGGGTVLLARGSLAADLGLPVLGVIGFAESFADGAHTSIPAPGLGALGAARDGVESRLAVALRSVGVSADEISIISKHDTSTNANDPNESDLHERIASAIGRADGNPMYVISQKSLTGHAKGGAAAFQMIGLTQVLRSGLVPANRALDCVDPVLSKHSHLVWLRKPLDLRAKAPKAGLVTSLGFGHVSALVAIVHPDAFYEAVRVARGAEAADVWRASAIAREEAGLRTIVAGMHGGVLYERPVERNLGVHGDAAKEVEAAVLLDSRARLVDGVLRAEG

SEQ ID NO: 9 - DNA, 72 bp, Corynebacterium glutamicum
gtgaccgaattgagcaggaacttcgattcccgcgcccgcctagttgacggtgtcctccgcgccgaaggctag

SEQ ID NO: 10 - protein, 23 aa, Corynebacterium glutamicum
MTELSRNFDSRARLVDGVLRAEG

SEQ ID NO: 11 - DNA, 247 bp, Corynebacterium glutamicum
cacacccaagagctaaaaattcatatagttaagacaacattttggctgtaaaagacagccgtaaaaacctcttgctcgtgtcaattgttcttatcggaatgtggcttgggcgattgttatgcaaaagttgttaggttttttgcggggttgtttaacccccaaatgagggaagatagtaaccttgaactctatgagcacaggtttaacagctaagaccggagccgctcgagtgggccgaacaaatatg

SEQ ID NO: 12 - DNA, 247 bp, Corynebacterium glutamicum
cacacccaagagctaaaaattcatatagttaagacaacattttggctgtaaaagacagccgtaaaaacctcttgctcgtgtcaattgttcttatcggaatgtggcttgggcgattgttatgcaaaagttgttaggttttttgcggggttgtttaacccccaaatgagggaagaaggtaaccttgaactctatgagcacaggtttaacagctaagaccggagccgctcgagtgggccgaacaaatatg

SEQ ID NO: 13 - DNA, 29 bp, Corynebacterium glutamicum
ccccagaatagtcgtaagtaagcatatct

SEQ ID NO: 14 - DNA, 29 bp, Corynebacterium glutamicum
ccccagaatatgagtaagtcctcatatct

SEQ ID NO: 15 - DNA, 29 bp, Corynebacterium glutamicum
tttcaaaacatgaccattagtagcccttt

SEQ ID NO: 16 - DNA, 29 bp, Corynebacterium glutamicum
tttcaaaacatgaccatttcctcaccttt

SEQ ID NO: 17 - DNA, 1212 bp, Aloe arborescens
atgtcctccttgtccaactccttgccactgatggaagatgtgcagggcatccgcaaggcacagaaggcagacggcaccgcaaccgtgatggcaatcggcaccgctcatccaccacacattttcccacaggatacctacgcagatgtgtactttcgcgcaaccaactccgaacacaaggtggaactgaagaagaagttcgatcacatctgcaagaaaaccatgatcggcaagcgctacttcaactacgatgaagagttcctgaagaagtacccaaacatcacctcctacgatgagccatccttgaacgatcgccaggatatctgcgtgccaggcgtcccagcactgggcaccgaagcagcagtgaaggcaatcgaagaatggggtcgcccaaagtccgaaatcacccacctggtgttctgcacctcttgcggtgtggatatgccatccgcagatttccagtgcgcaaagctgctgggcctgcacgcaaacgtgaacaagtactgcatctacatgcagggctgctacgcaggcggaaccgtcatgcgctacgcaaaggatctggcagaaaacaaccgtggcgcacgcgtcctggtggtgtgcgcagaactgaccattatgatgcttcgcgcaccaaacgaaacccacctcgataacgccatcggcatctccctgttcggtgatggcgcagcagcactgatcatcggctccgatccaatcatcggcgtggaaaagccaatgttcgaaatcgtgtgcaccaagcagaccgtgattccaaacaccgaggatgtgatccacctccacctccgcgaaaccggcatgatgttctacctgtccaagggctccccaatgaccatctccaacaacgtggaagcatgcctgatcgatgtgttcaagtccgtgggcatcaccccaccagaagattggaactccctcttctggattccacatccaggcggtcgcgcaatcctggatcaggtggaagcaaagctgaagctgcgtccagaaaagtttcgcgcagcacgcaccgtgctgtgggattacggcaacatggtgtccgcatccgtgggctacatcctggatgaaatgcgtcgcaagtccgcagcaaagggcctcgaaacctacggcgaaggcctggaatggggtgtgctgctcggcttcggtccaggcatcaccgtggaaaccatcctgctgcactccctgccactcatgtaa

SEQ ID NO: 18 - protein, 403 aa, Aloe arborescens
MSSLSNSLPLMEDVQGIRKAQKADGTATVMAIGTAHPPHIFPQDTYADVYFRATNSEHKVELKKKFDHICKKTMIGKRYFNYDEEFLKKYPNITSYDEPSLNDRQDICVPGVPALGTEAAVKAIEEWGRPKSEITHLVFCTSCGVDMPSADFQCAKLLGLHANVNKYCIYMQGCYAGGTVMRYAKDLAENNRGARVLVVCAELTIMMLRAPNETHLDNAIGISLFGDGAAALIIGSDPIIGVEKPMFEIVCTKQTVIPNTEDVIHLHLRETGMMFYLSKGSPMTISNNVEACLIDVFKSVGITPPEDWNSLFWIPHPGGRAILDQVEAKLKLRPEKFRAARTVLWDYGNMVSASVGYILDEMRRKSAAKGLETYGEGLEWGVLLGFGPGITVETILLHSLPLM

SEQ ID NO: 19 - DNA, 1182 bp, Corynebacterium glutamicum
atggaagatgtgcagggcatccgcaaggcacagaaggcagacggcaccgcaaccgtgatggcaatcggcaccgctcatccaccacacattttcccacaggatacctacgcagatgtgtactttcgcgcaaccaactccgaacacaaggtggaactgaagaagaagttcgatcacatctgcaagaaaaccatgatcggcaagcgctacttcaactacgatgaagagttcctgaagaagtacccaaacatcacctcctacgatgagccatccttgaacgatcgccaggatatctgcgtgccaggcgtcccagcactgggcaccgaagcagcagtgaaggcaatcgaagaatggggtcgcccaaagtccgaaatcacccacctggtgttctgcacctcttgcggtgtggatatgccatccgcagatttccagtgcgcaaagctgctgggcctgcacgcaaacgtgaacaagtactgcatctacatgcagggctgctacgcaggcggaaccgtcatgcgctacgcaaaggatctggcagaaaacaaccgtggcgcacgcgtcctggtggtgtgcgcagaactgaccattatgatgcttcgcgcaccaaacgaaacccacctcgataacgccatcggcatctccctgttcggtgatggcgcagcagcactgatcatcggctccgatccaatcatcggcgtggaaaagccaatgttcgaaatcgtgtgcaccaagcagaccgtgattccaaacaccgaggatgtgatccacctccacctccgcgaaaccggcatgatgttctacctgtccaagggctccccaatgaccatctccaacaacgtggaagcatgcctgatcgatgtgttcaagtccgtgggcatcaccccaccagaagattggaactccctcttctggattccacatccaggcggtcgcgcaatcctggatcaggtggaagcaaagctgaagctgcgtccagaaaagtttcgcgcagcacgcaccgtgctgtgggattacggcaacatggtgtccgcatccgtgggctacatcctggatgaaatgcgtcgcaagtccgcagcaaagggcctcgaaacctacggcgaaggcctggaatggggtgtgctgctcggcttcggtccaggcatcaccgtggaaaccatcctgctgcactccctgccactcatgtaa

SEQ ID NO: 20 - protein, 393 aa, Aloe arborescens
MEDVQGIRKAQKADGTATVMAIGTAHPPHIFPQDTYADVYFRATNSEHKVELKKKFDHICKKTMIGKRYFNYDEEFLKKYPNITSYDEPSLNDRQDICVPGVPALGTEAAVKAIEEWGRPKSEITHLVFCTSCGVDMPSADFQCAKLLGLHANVNKYCIYMQGCYAGGTVMRYAKDLAENNRGARVLVVCAELTIMMLRAPNETHLDNAIGISLFGDGAAALIIGSDPIIGVEKPMFEIVCTKQTVIPNTEDVIHLHLRETGMMFYLSKGSPMTISNNVEACLIDVFKSVGITPPEDWNSLFWIPHPGGRAILDQVEAKLKLRPEKFRAARTVLWDYGNMVSASVGYILDEMRRKSAAKGLETYGEGLEWGVLLGFGPGITVETILLHSLPLM

SEQ ID NO: 21 - DNA, 1635 bp, Corynebacterium glutamicum
atgggcgattgcgtggcaccaaaagaagatctcatcttccgctccaagctgccagatatctacatcccaaagcacctcccactgcacacctactgcttcgaaaacatctccaaggtgggcgataagtcctgcctgatcaacggtgcaaccggtgaaaccttcacctactcccaggtggaactgctgtcccgcaaggtggcatccggtctgaacaagctgggcatccagcagggcgataccatcatgctgctgctgccaaactccccagaatacttcttcgcattcctgggtgcatcctaccgtggcgcaatctccactatggcaaacccattcttcacctccgcagaagtgatcaagcagctgaaggcatccctggcaaagctgatcatcacccaggcatgctacgtggataaggtgaaggattacgcagcagaaaagaacatccagatcatctgcatcgatgatgcaccacaggattgcctgcacttctccaagctgatggaagcagatgaatccgaaatgccagaagtggtgatcgattccgatgatgtggtggcactgccatactcctccggcaccaccggtctgccaaagggcgtgatgctgacccacaagggcctggtgacctccgtggcacagcaggtcgatggcgataacccaaacctgtacatgcactccgaagatgtgatgatctgcatcctgccactgttccacatctactccctgaacgcagtgctgtgctgcggtctgcgtgcaggcgtgaccatcctgatcatgcagaagttcgatatcgtcccattcctggaactgatccagaagtacaaggtcaccatcggtccattcgtgccaccaatcgtgctggcaatcgcaaagtccccagtggtggataagtacgatctgtcctccgtgcgtaccgtcatgtccggtgcagcaccactgggcaaagaactggaagatgcagttcgcgcaaagttcccaaacgcaaagctgggccagggctacggcatgaccgaagcaggcccagtgctggcaatgtgcctggcattcgcaaaagaaccatacgaaatcaagtccggtgcatgcggcaccgtggtgcgcaacgcagaaatgaagatcgtggacccagaaaccaacgcatccctgccacgcaaccagcgtggcgaaatctgcatccgtggcgatcagatcatgaagggctacctgaacgatccagaatccacccgcaccaccatcgatgaagaaggctggctgcacaccggtgatatcggcttcatcgatgatgatgatgaactgttcatcgtggatcgcctgaaagaaatcatcaagtacaagggcttccaggtggcaccagcagaactggaagcactgctgctgacccacccaaccatctccgatgcagcagtggtgccaatgatcgatgaaaaggcaggcgaagtgccagtggcattcgtggtccgcaccaacggcttcaccaccaccgaagaagaaatcaagcagttcgtgtccaagcaggtcgtgttctacaagcgcatcttccgcgtgttcttcgtggatgcaatcccaaaatccccatccggcaagatcctgcgcaaggaccttcgcgcaaagatcgcatccggtgatctgccaaagtaa

SEQ ID NO: 22 - protein, 544 aa, Petroselinum crispum
MGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASLAKLIITQACYVDKVKDYAAEKNIQIICIDDAPQDCLHFSKLMEADESEMPEVVIDSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRAKIASGDLPK

SEQ ID NO: 23 - DNA, 1170 bp, Corynebacterium glutamicum
atggtgtccgtgtccggcatccgcaaggtgcaacgcgctgaaggcccagcaaccgtgctggcaatcggcaccgcaaacccaccaaactgcgtggatcagtccacctacgcagattactacttccgcgtgaccaactccgaacacatgaccgatctgaagaagaagttccagcgcatctgcgagcgcacccagatcaagaaccgccacatgtacctgaccgaagaaatcctgaaagaaaacccaaacatgtgcgcatacaaggcaccatccctggatgcacgcgaagatatgatgatccgcgaagtgccacgcgtcggcaaagaagcagcaaccaaggcaatcaaagaatggggtcagccaatgtccaagatcacccacctgattttctgcaccacctccggtgtggcactgccaggcgtggattacgaactgatcgtgctgctgggcctggacccatccgtgaagcgctacatgatgtaccaccagggctgctttgccggtggcaccgtgctgcgcctggcaaaggatctggcagaaaacaacaaggatgcacgcgtcctgatcgtgtgctccgaaaacacctccgtgaccttccgtggcccatccgaaaccgatatggattccctggtgggccaggcactgttcgcagatggcgcagcagcaatcatcatcggctccgatccagtcccagaagtggaaaacccactgttcgaaatcgtgtccaccgatcagcagctggtgccaaactcccacggtgcaatcggtggcctgctgcgcgaagtgggcctgaccttctacctgaacaagtccgtgccagatatcatctcccagaacatcaacgatgcactgtccaaggcattcgatccactgggcatctccgattacaactccatcttctggatcgcacacccaggcggtcgcgcaatcctggatcaggtggaagaaaaggtgaacctgaagccagaaaagatgaaggcaacccgtgatgtgctgtccaactacggcaacatgtcctccgcatgcgtgttcttcatcatggatctgatgcgcaagaagtccctggaagcaggcctcaagaccaccggtgaaggcctggattggggtgtgctgttcggcttcggtccaggcctgaccatcgaaaccgtggtgctgcgctctatggcaatctaa

SEQ ID NO: 24 - protein, 389 aa, Arachis hypgea
MVSVSGIRKVQRAEGPATVLAIGTANPPNCVDQSTYADYYFRVTNSEHMTDLKKKFQRICERTQIKNRHMYLTEEILKENPNMCAYKAPSLDAREDMMIREVPRVGKEAATKAIKEWGQPMSKITHLIFCTTSGVALPGVDYELIVLLGLDPSVKRYMMYHQGCFAGGTVLRLAKDLAENNKDARVLIVCSENTSVTFRGPSETDMDSLVGQALFADGAAAIIIGSDPVPEVENPLFEIVSTDQQLVPNSHGAIGGLLREVGLTFYLNKSVPDIISQNINDALSKAFDPLGISDYNSIFWIAHPGGRAILDQVEEKVNLKPEKMKATRDVLSNYGNMSSACVFFIMDLMRKKSLEAGLKTTGEGLDWGVLFGFGPGLTIETVVLRSMAI

SEQ ID NO: 25 - DNA, 1170 bp, Corynebacterium glutamicum
atggtgaccgtggaagaataccgcaaggcacaacgcgctgaaggcccagcaaccgtgatggcaatcggcaccgcaaccccaaccaactgcgtggatcagtccacctacccagattactacttccgcatcaccaactccgaacacaagaccgatctgaaagaaaagttcaagcgcatgtgcgaaaagtccatgatcaagaaacgctacatgcacctgaccgaagaaatcctgaaagaaaacccatccatgtgcgaatacatggcaccatccctggatgcacgccaggatatcgtggtggtggaagtgccaaagctgggcaaagaagcagcacagaaagcaatcaaagaatggggtcagccaaagtccaagatcacccacctggtgttctgcaccacctccggtgtggatatgccaggctgcgattaccagctgaccaagctgctgggcctgcgtccatccgtgaagcgcctgatgatgtaccagcagggttgctttgccggtggcaccgtgctgcgcctggcaaaggatctggcagaaaacaacaagggtgcacgcgtcctcgtggtgtgctccgaaatcaccgcagtgaccttccgtggcccaaacgatacccacctggattccctggtgggccaggcactgttcggtgatggcgcaggcgcaatcatcatcggctccgatccaatcccaggcgtggaacgcccactgttcgaactggtgtccgcagcacagaccctgctgccagattcccacggtgcaatcgatggccacctccgcgaagtgggcctgaccttccacctcctgaaggatgtgccaggcctgatctccaagaacatcgaaaagtccctggaagaagcattcaagccactgggcatctccgattggaactccctgttctggatcgcacacccaggcggtccagcaatcctggatcaggtggaaatcaagctgggcctgaagccagaaaagctgaaggcaacccgcaacgtgctgtccgattacggcaacatgtcctccgcatgcgtgctgttcatcctggatgaaatgcgcaaggcatccgcaaaagaaggcctgggcaccaccggtgaaggcctggaatggggtgtgctgttcggcttcggtcctggcctgaccgtggaaaccgtggtgctgcactccgtggcaacctaa

SEQ ID NO: 26 - protein, 389 aa, Petunia x hybrida
MVTVEEYRKAQRAEGPATVMAIGTATPTNCVDQSTYPDYYFRITNSEHKTDLKEKFKRMCEKSMIKKRYMHLTEEILKENPSMCEYMAPSLDARQDIVVVEVPKLGKEAAQKAIKEWGQPKSKITHLVFCTTSGVDMPGCDYQLTKLLGLRPSVKRLMMYQQGCFAGGTVLRLAKDLAENNKGARVLVVCSEITAVTFRGPNDTHLDSLVGQALFGDGAGAIIIGSDPIPGVERPLFELVSAAQTLLPDSHGAIDGHLREVGLTFHLLKDVPGLISKNIEKSLEEAFKPLGISDWNSLFWIAHPGGPAILDQVEIKLGLKPEKLKATRNVLSDYGNMSSACVLFILDEMRKASAKEGLGTTGEGLEWGVLFGFGPGLTVETVVLHSVAT

SEQ ID NO: 27 - DNA, 726 bp, Corynebacterium glutamicum
atgtccccaccagtgtccgtgaccaagatgcaggtcgaaaactacgcattcgcaccaaccgtgaacccagcaggctccaccaacaccctgttcctcgcaggcgcaggtcaccgtggcctcgaaatcgaaggcaagttcgtgaagttcaccgcaatcggcgtgtacctggaagaatccgcaatcccattcctggcagaaaagtggaagggcaagaccccacaagaactgaccgattccgtcgagttcttccgcgacgtggtgaccggtccattcgaaaagttcacccgtgtgaccatgatcctgccactgaccggcaagcagtactccgaaaaggtggcagaaaactgcgtcgcacactggaagggcatcggcacctacaccgatgatgaaggtcgcgcaatcgaaaagttcctggatgtgttccgctccgaaaccttcccaccaggcgcatccatcatgttcacccagtccccactgggcctgctgaccatctccttcgcaaaggatgattccgtgaccggcaccgcaaacgcagtgatcgaaaacaagcagctgtccgaagcagtgctggaatccatcatcggcaagcacggcgtgtccccagcagcaaagtgctccgtggcagaacgcgtggcagaactgctgaagaagtcctacgccgaagaggcatccgtgttcggcaagccagaaaccgaaaagtccaccatcccagtgatcggcgtgtaa

SEQ ID NO: 28 - protein, 241 aa, Petunia x hybrid
MSPPVSVTKMQVENYAFAPTVNPAGSTNTLFLAGAGHRGLEIEGKFVKFTAIGVYLEESAIPFLAEKWKGKTPQELTDSVEFFRDVVTGPFEKFTRVTMILPLTGKQYSEKVAENCVAHWKGIGTYTDDEGRAIEKFLDVFRSETFPPGASIMFTQSPLGLLTISFAKDDSVTGTANAVIENKQLSEAVLESIIGKHGVSPAAKCSVAERVAELLKKSYAEEASVFGKPETEKSTIPVIGV

SEQ ID NO: 29 - DNA, 1047 bp, Escherichia coli
atgaacagaactgacgaactccgtactgcgcgtattgagagcctggtaacgcccgccgaactcgcgctacggtatcccgtaacgcctggcgtcgccacccatgtcaccgactcccgccgcagaattgaaaaaatactgaatggtgaagataagcgactgttggtcattattggcccctgctcgatccacgatctcaccgctgcaatggagtacgccacccgtctgcagtcgctgcgcaaccagtaccagtcacggctggaaatcgtaatgcgcacctattttgaaaaaccacgaactgttgtcggctggaaaggactaatctccgatccagatttaaacggcagctatcgggtaaatcacggtctggagctggcgcgcaaattacttttacaggtaaatgagctgggcgtcccaaccgcgaccgagttcctcgatatggtgaccggtcagtttattgctgatttaatcagttggggcgcgattggcgcacgtactaccgaaagtcagatccaccgcgaaatggcttcggcactctcctgtccggtaggttttaaaaatggtaccgatggcaatacgcggattgctgtggatgctatccgcgcagcccgcgccagccatatgttcctctcgccagacaaaaatggtcagatgaccatctatcagaccagcggcaacccgtatggccacattattatgcgtggcggcaaaaaaccgaattatcatgccgatgatatcgccgcagcctgcgatacgctgcacgagtttgatttacctgaacatctggtggtggatttcagccacggtaactgccagaagcagcaccgtcgccagttagaagtttgtgaggatatttgtcagcaaatccgcaatggctctacggcgattgctggaattatggcggaaagtttcctgcgcgaaggaacgcaaaaaatcgtcggcagtcagccgctcacttacggtcaatccattaccgacccgtgtctgggctgggaggataccgaacgcctggtcgaaaaactcgcctctgcggtagatacccgcttctaa

SEQ ID NO: 30 - protein, 348 aa, Escherichia coli
MNRTDELRTARIESLVTPAELALRYPVTPGVATHVTDSRRRIEKILNGEDKRLLVIIGPCSIHDLTAAMEYATRLQSLRNQYQSRLEIVMRTYFEKPRTVVGWKGLISDPDLNGSYRVNHGLELARKLLLQVNELGVPTATEFLDMVTGQFIADLISWGAIGARTTESQIHREMASALSCPVGFKNGTDGNTRIAVDAIRAARASHMFLSPDKNGQMTIYQTSGNPYGHIIMRGGKKPNYHADDIAAACDTLHEFDLPEHLVVDFSHGNCQKQHRRQLEVCEDICQQIRNGSTAIAGIMAESFLREGTQKIVGSQPLTYGQSITDPCLGWEDTERLVEKLASAVDTRF

SEQ ID NO: 31 - DNA, 1521 bp, Corynebacterium glutamicum
atgaacaccatcaacgaatacctgtccctggaagagttcgaagcaatcatcttcggcaaccagaaagtgaccatctccgatgtggtggtgaaccgcgtgaacgaatccttcaacttcctgaaagagttctccggcaacaaggtgatctacggcgtgaacaccggcttcggtccaatggcacagtaccgcatcaaagaatccgatcagatccagctccagtacaacctgatccgctcccactcctccggcaccggcaagccactgtccccagtgtgcgcaaaggcagcaatcctggcacgcctgaacaccctgtccctgggcaactccggtgtgcacccatccgtgatcaacctgatgtccgaactgatcaacaaggatatcaccccactgatcttcgaacacggtggcgttggcgcatccggtgatctggtgcagctgtcccacctggcactggtgctgatcggcgaaggcgaagtgttctacaagggcgaacgccgtccaaccccagaagtgttcgaaatcgaaggcctgaagccaatccaggtggaaatccgcgaaggcctggcactgatcaacggcacctccgtgatgaccggcatcggcgtggtgaacgtgtaccacgcaaagaagctgctggattggtccctgaagtcctcctgcgcaatcaacgaactggtgcaggcatacgatgatcacttctccgcagaactgaaccagaccaagcgccacaagggccagcaagaaatcgcactgaagatgcgccagaacctgtccgattccaccctgatccgcaagcgcgaagatcacctgtactccggtgaaaacaccgaagaaatcttcaaagaaaaggtgcaagaatactactccctgcgctgcgtgccacagatcctgggtccagtgctggaaaccatcaacaacgtggcatccatcctggaagatgagttcaactccgcaaacgataacccaatcatcgatgtgaagaaccagcacgtctaccacggtggcaacttccacggcgattacatctccctggaaatggataagctgaagatcgtgatcaccaagctgaccatgctggcagaacgccagctgaactacctgctgaactccaagatcaacgaactcctgccaccattcgtgaacctgggcaccctgggcttcaacttcggcatgcagggcgtgcagttcaccgcaacctccaccaccgcagaatcccagatgctgtccaacccaatgtacgtgcactccatcccaaacaacaacgataaccaggatatcgtgtctatgggcaccaactccgcagtgatcacctccaaggtgatcgaaaacgcattcgaagtgctggcaatcgaaatgatcaccatcgtgcaggcaatcgactacctgggccagaaggataagatctcctccgtgtccaagaagtggtacgacgaaatccgcaacatcatcccaaccttcaaagaagatcaggtgatgtacccattcgtgcagaaggtgaaggatcacctgatcaacaactaa

SEQ ID NO: 32 - protein, 506 aa, Flavobacerium johnsoniae
MNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 33 - DNA, 29 bp, Unknown
  Primer
tgctctagagcatgaactgggacttgaag

SEQ ID NO: 34 - DNA, 43 bp, Unknown
  Primer
tatgcatgtttctcgagtgggccgaacaaatatgtttgaaagg

SEQ ID NO: 35 - DNA, 43 bp, Unknown
  Primer
cccactcgagaaacatgcatagcgttttcaatagttcggtgtc

SEQ ID NO: 36 - DNA, 38 bp, Unknown
  Primer
ccccccggggggcctagggaaaggatgatctcgtagcc

SEQ ID NO: 37 - DNA, 47 bp, Unknown
  Primer
ccaatgcattggttctgcagttatcacacccaagagctaaaaattca

SEQ ID NO: 38 - DNA, 35 bp, Unknown
  Primer
ccgctcgagcggctccggtcttagctgttaaacct

SEQ ID NO: 39 - DNA, 22 bp, Unknown
  Primer
atgagtccgaaggttgctgcat

SEQ ID NO: 40 - DNA, 21 bp, Unknown
  Primer
tcgagtgggttcagctggtcc

SEQ ID NO: 41 - DNA, 24 bp, Unknown
  Primer
cgccagggttttcccagtcacgac

SEQ ID NO: 42 - DNA, 23 bp, Unknown
  Primer
cacaggaaacagctatgaccatg

SEQ ID NO: 43 - DNA, 38 bp, Unknown
  Primer
atccccgggtaccgagctcgaaccagcgcgcgttcgtg

SEQ ID NO: 44 - DNA, 53 bp, Unknown
  Primer
ttacgactattctgggggaattcttctgttttaggcaggaaatatggcttatg

SEQ ID NO: 45 - DNA, 59 bp, Unknown
  Primer
agaagaattcccccagaatagtcgtaagtaagcatatctggttgagttcttcggggttg

SEQ ID NO: 46 - DNA, 38 bp, Unknown
  Primer
ttgtaaaacgacggccagtggccttggcggtatctgcg

SEQ ID NO: 47 - DNA, 25 bp, Unknown
  Primer
gttcggccactccgatgtccgcctg

SEQ ID NO: 48 - DNA, 23 bp, Unknown
  Primer
gccttgatggcgattgggagacc

SEQ ID NO: 49 - DNA, 43 bp, Unknown
  Primer
atccccgggtaccgagctcgtcattcaacgcatccatgacagc

SEQ ID NO: 50 - DNA, 38 bp, Unknown
  Primer
ctaatggtcatgttttgaaatcgtagcggtaggcgggg

SEQ ID NO: 51 - DNA, 59 bp, Unknown
  Primer
accgctacgatttcaaaacatgaccattagtagccctttgattgacgtcgccaaccttc

SEQ ID NO: 52 - DNA, 42 bp, Unknown
  Primer
ttgtaaaacgacggccagtgcgccagaagcctgaatgttttg

SEQ ID NO: 53 - DNA, 28 bp, Unknown
  Primer
ggctgatattagtgccccaaccgatgac

SEQ ID NO: 54 - DNA, 25 bp, Unknown
  Primer
gatcacgtctgggccggtaacgaac

SEQ ID NO: 55 - DNA, 45 bp, Unknown
  Primer
atccccgggtaccgagctcgaattcgcgatttcgatgcctggatg

SEQ ID NO: 56 - DNA, 30 bp, Unknown
  Primer
cgcgggaatcgaagttcctgctcaattcgg

SEQ ID NO: 57 - DNA, 28 bp, Unknown
  Primer
caggaacttcgattcccgcgcccgccta

SEQ ID NO: 58 - DNA, 60 bp, Unknown
  Primer
ttgtaaaacgacggccagtgaattcgatactgcaatatcaaaccaagatctccattctcc

SEQ ID NO: 59 - DNA, 23 bp, Unknown
  Primer
ggaggatacatccacggtcattg

SEQ ID NO: 60 - DNA, 25 bp, Unknown
  Primer
cgctatgagttcaggatgttgatcg

SEQ ID NO: 61 - DNA, 47 bp, Unknown
  Primer
gtaccgctgcgatggcaaccaagaaagcaaccacctcccaggccgtc

SEQ ID NO: 62 - DNA, 47 bp, Unknown
  Primer
gacggcctgggaggtggttgctttcttggttgccatcgcagcggtac

SEQ ID NO: 63 - DNA, 47 bp, Unknown
  Primer
aaaacctgcaggggctgagctcgctggtggcggacaggttaccccag

SEQ ID NO: 64 - DNA, 46 bp, Unknown
  Primer
ggggtctagaacgtccttatcaatgacgggcacaaagttcacaggc

SEQ ID NO: 65 - DNA, 47 bp, Unknown
  Primer
cctcacccagttcacccaggtggacatggcaactctgggcgttgctc

SEQ ID NO: 66 - DNA, 47 bp, Unknown
  Primer
gagcaacgcccagagttgccatgtccacctgggtgaactgggtgagg

SEQ ID NO: 67 - DNA, 48 bp, Unknown
  Primer
aaaacctgcagggttgcacctgaatccatgcgcccattcgctgtgatc

SEQ ID NO: 68 - DNA, 45 bp, Unknown
  Primer
ggaatctagatcggcggaagcagccttgaaatcagccaagatctc

SEQ ID NO: 69 - DNA, 48 bp, Unknown
  Primer
cattcgcggcacctcgcgtgtccgaatccatggcagatgcaggcccac

SEQ ID NO: 70 - DNA, 48 bp, Unknown
  Primer
gtgggcctgcatctgccatggattcggacacgcgaggtgccgcgaatg

SEQ ID NO: 71 - DNA, 47 bp, Unknown
  Primer
aaaacctgcaggttggccacgtcaggttgcaccaagcttcgatgaag

SEQ ID NO: 72 - DNA, 46 bp, Unknown
  Primer
aaaatctagaccgagctcgccggcgccaacgatgacgaccatctcg

SEQ ID NO: 73 - DNA, 41 bp, Unknown
  Primer
agtccgacttcgttgtcgcatccggcttcgatgccctgtcc

SEQ ID NO: 74 - DNA, 41 bp, Unknown
  Primer
ggacagggcatcgaagccggatgcgacaacgaagtcggact

SEQ ID NO: 75 - DNA, 46 bp, Unknown
  Primer
aaaacctgcaggcactgacctacgtcgactccgagccagaactcac

SEQ ID NO: 76 - DNA, 44 bp, Unknown
  Primer
ggggtctagatgcgcagccagacgaggtgggaatgcttggacag

SEQ ID NO: 77 - DNA, 33 bp, Unknown
  Primer
ctctctagagcggtggcgatgatgatcttcgag

SEQ ID NO: 78 - DNA, 60 bp, Unknown
  Primer
aagcatatgagccaagtactatcaacgcgtcagggcgacttttccattgagagacatttc

SEQ ID NO: 79 - DNA, 60 bp, Unknown
  Primer
ctgacgcgttgatagtacttggctcatatgcttttcctcacccgcttctacgcttaaaag

SEQ ID NO: 80 - DNA, 33 bp, Unknown
  Primer
gacgaattcgtgtggccaccacctcaatctgtg

SEQ ID NO: 81 - DNA, 23 bp, Unknown
  Primer
agagattcaccctcggcgatgag

SEQ ID NO: 82 - DNA, 21 bp, Unknown
  Primer
gacccgcaatggtgtcgccag

SEQ ID NO: 83 - DNA, 31 bp, Unknown
  Primer
acatctagaggtcggcgaatcaagctccatg

SEQ ID NO: 84 - DNA, 76 bp, Unknown
  Primer
cgtctcgagttcacatatgcaacgcgtgctcaagatgacaatatcttgagggttcattttttgatccttaatttag

SEQ ID NO: 85 - DNA, 58 bp, Unknown
  Primer
ttgagcacgcgttgcatatgtgaactcgagacggtcggtggaggcgaccagggataac

SEQ ID NO: 86 - DNA, 38 bp, Unknown
  Primer
tctgaattcatcaaggccaatcatgatgagtgcgaaac

SEQ ID NO: 87 - DNA, 30 bp, Unknown
  Primer
aagaggagttgatgggatggtcgaacaatc

SEQ ID NO: 88 - DNA, 25 bp, Unknown
  Primer
gttggcatgccagctttgtgggatg

SEQ ID NO: 89 - DNA, 67 bp, Unknown
  Primer
tcctacgcgttaatacgactcactatagggagatcaaggaggcggacaatgggcgattgcgtggcac

SEQ ID NO: 90 - DNA, 41 bp, Unknown
  Primer
ggacgttcatatgttactttggcagatcaccggatgcgatc

SEQ ID NO: 91 - DNA, 29 bp, Unknown
  Primer
acgaagctttgtccggcatgctggctgac

SEQ ID NO: 92 - DNA, 64 bp, Unknown
  Primer
tgcgcatatgtggccgtctagatacgcgtacgtcaaacaaacagtggcaatggatgtacgcatg

SEQ ID NO: 93 - DNA, 61 bp, Unknown
  Primer
acgtacgcgtatctagacggccacatatgcgcaatcgagcggggaatcccaaactagcatc

SEQ ID NO: 94 - DNA, 33 bp, Unknown
  Primer
tatggatcctacgcctgtacaccgtcgcacgtc

SEQ ID NO: 95 - DNA, 32 bp, Unknown
  Primer
gtgaacattgtgtttactgtgtgggcactgtc

SEQ ID NO: 96 - DNA, 31 bp, Unknown
  Primer
tgatgttcaggccgttgaagccaaggtagag

SEQ ID NO: 97 - DNA, 34 bp, Unknown
  Primer
cacaagcttccacacgatgaaaatcaatccgcag

SEQ ID NO: 98 - DNA, 58 bp, Unknown
  Primer
tgcggtaccctcgcatatgatatctcgagagctaattgccactggtacgtggttcatg

SEQ ID NO: 99 - DNA, 63 bp, Unknown
  Primer
agctctcgagatatcatatgcgagggtaccgcagacctaccacgcttcgaggtataaacgctc

SEQ ID NO: 100 - DNA, 31 bp, Unknown
  Primer
agtgaattccaaggaaggcggttgctactgc

SEQ ID NO: 101 - DNA, 29 bp, Unknown
  Primer
taaatggtggagataccaaactgtgaagc

SEQ ID NO: 102 - DNA, 25 bp, Unknown
  Primer
cgagttcttcttcgtgttcgcgatc

SEQ ID NO: 103 - DNA, 59 bp, Unknown
  Primer
ctcggatccaaggaggtcatatcatgaacagaacgacgaactccgtactgcgcgtattg

SEQ ID NO: 104 - DNA, 44 bp, Unknown
  Primer
tacgctcttctgatttagaagcgggtatctaccgcagaggcgag

SEQ ID NO: 105 - DNA, 66 bp, Unknown
  Primer
ttcgctcttcaatctggcaaggagggatccgtatgaacaccatcaacgaatacctgtccctggaag

SEQ ID NO: 106 - DNA, 45 bp, Unknown
  Primer
atcgaattcttagttgttgatcaggtgatccttcaccttctgcac

SEQ ID NO: 107 - DNA, 38 bp, Unknown
  Primer
gcaaatattctgaaatgagctgttgacaattaatcatc

SEQ ID NO: 108 - DNA, 38 bp, Unknown
  Primer
cgttctgatttaatctgtatcaggctgaaaatcttctc

SEQ ID NO: 109 - DNA, 44 bp, Unknown
  Primer
ataccatggtaaggaggacagctatggtgtccgtgtccggcatc

SEQ ID NO: 110 - DNA, 37 bp, Unknown
  Primer
ctcggtacctttagattgccatagagcgcagcaccac

SEQ ID NO: 111 - DNA, 42 bp, Unknown
  Primer
agcggtacctaaggaggtggacaatgggcgattgcgtggcac

SEQ ID NO: 112 - DNA, 59 bp, Unknown
  Primer
ctgggatccaggactagtttccagagtactattactttggcagatcaccggatgcgatc

SEQ ID NO: 113 - DNA, 22 bp, Unknown
  Primer
ccctcaagacccgtttagaggc

SEQ ID NO: 114 - DNA, 32 bp, Unknown
  Primer
ttaatacgactcactataggggaattgtgagc

SEQ ID NO: 115 - DNA, 49 bp, Unknown
  Primer
gtatctagaaaggaggtcgaagatggtgaccgtggaagaataccgcaag

SEQ ID NO: 116 - DNA, 33 bp, Unknown
  Primer
ctcccatggttaggttgccacggagtgcagcac

SEQ ID NO: 117 - DNA, 54 bp, Unknown
  Primer
ctcccatggtgctaaaggaggtcgaagatgtccccaccagtgtccgtgaccaag

SEQ ID NO: 118 - DNA, 34 bp, Unknown
  Primer
ctgggatccttacacgccgatcactgggatggtg

SEQ ID NO: 119 - DNA, 57 bp, Unknown
  Primer
actttaagaaggagatataccatggtaaggaggacagctatgtcctccttgtccaac

SEQ ID NO: 120 - DNA, 43 bp, Unknown
  Primer
ccaggactagtttccagagtactattacatgagtggcagggag

SEQ ID NO: 121 - DNA, 57 bp, Unknown
  Primer
actttaagaaggagatataccatggtaaggaggacagctatggaagatgtgcagggc